(19) United States
(12) Patent Application Publication
(10) Pub. No.: US 2008/0221127 A1
(43) Pub. Date: Sep. 11, 2008

(54) PPAR ACTIVE COMPOUNDS

(75) Inventors: Jack Lin, Hercules, CA (US); Patrick Womack, Berkeley, CA (US); Shenghua Shi, San Diego, CA (US); Chao Zhang, Moraga, CA (US); Dean R. Artis, Kensington, CA (US); Prabha N. Ibrahim, Mountain View, CA (US); Weiru Wang, Lafayette, CA (US)

Correspondence Address: FOLEY & LARDNER LLP, P.O. BOX 80278, SAN DIEGO, CA, 92138-0278, US

(73) Assignee: Plexxikon Inc

(21) Appl. No.: 12/043,095

(22) Filed: Mar. 5, 2008

Related U.S. Application Data
(60) Provisional application No. 60/893,875, filed on Mar. 8, 2007.

(51) Int. Cl. A61K 31/50; C07C 63/00; A61K 31/19; C07D 231/12; A61K 31/415; A61P 3/04; A61K 31/44; C07D 211/86; C07D 239/02

(52) U.S. Cl. 514/256; 562/405; 514/568; 548/376.1; 514/406; 514/345; 546/301; 544/315

(57) ABSTRACT

Compounds are described that are active on at least one of PPARα, PPARδ, and PPARγ, which are useful for therapeutic and/or prophylactic methods involving modulation of at least one of PPARα, PPARδ, and PPARγ.

### RELATED PATENT APPLICATIONS

This application claims priority to U.S. Provisional App. No. 60/893,875, entitled “PPAR Active Compounds”, filed Mar. 8, 2007, which is incorporated herein by reference in its entirety and for all purposes.

### FIELD OF THE INVENTION

The present invention relates to the field of modulators for members of the family of nuclear receptors identified as peroxisome proliferator-activated receptors.

### BACKGROUND OF THE INVENTION

The following description is provided solely to assist the understanding of the reader. None of the references cited or information provided is admitted to be prior art to the present invention. Each of the references cited herein is incorporated by reference in its entirety, to the same extent as if each reference were individually indicated to be incorporated by reference herein in its entirety.

The peroxisome proliferator-activated receptors (PPARs) form a subfamily in the nuclear receptor superfamily. Three isoforms, encoded by separate genes, have been identified thusfar: PPARγ, PPARα, and PPARδ.

There are two PPARγ isoforms expressed at the protein level in mouse and human, γ1 and γ2. They differ only in that the latter has 30 additional amino acids at its N terminus due to differential promoter usage within the same gene, and subsequent alternative RNA processing. PPARγ2 is expressed primarily in adipose tissue, while PPARγ1 is expressed in a broad range of tissues.

Murine PPARα was the first member of this nuclear receptor subclass to be cloned; it has since been cloned from humans. PPARα is expressed in numerous metabolically active tissues, including liver, kidney, heart, skeletal muscle, and brown fat. It is also present in monocytes, vascular endothelium, and vascular smooth muscle cells. Activation of PPARα induces hepatic peroxisome proliferation, hepatomegaly, and hepatocarcinogenesis in rodents. These toxic effects are not observed in humans, although the same compounds activate PPARα across species.

Human PPARδ was cloned in the early 1990s and subsequently cloned from rodents. PPARδ is expressed in a wide range of tissues and cells; with the highest levels of expression found in the digestive tract, heart, kidney, liver, adipose, and brain.

The PPARs are ligand-dependent transcription factors that regulate target gene expression by binding to specific peroxisome proliferator response elements (PPREs) in enhancer sites of regulated genes. PPARs possess a modular structure composed of functional domains that include a DNA binding domain (DBD) and a ligand binding domain (LBD). The DBD specifically binds PPREs in the regulatory region of PPAR-responsive genes. The DBD, located in the C-terminal half of the receptor, contains the ligand-dependent activation domain, AF-2. Each receptor binds to its PPRE as a heterodimer with a retinoid X receptor (RXR). Upon binding an agonist, the conformation of a PPAR is altered and stabilized such that a binding cleft, made up in part of the AF-2 domain, is created and recruitment of transcriptional coactivators occurs. Coactivators augment the ability of nuclear receptors to initiate the transcription process. The result of the agonist-induced PPAR-coactivator interaction at the PPRE is an increase in gene transcription. Downregulation of gene expression by PPARs appears to occur through indirect mechanisms. (Bergen, et al., Diabetes Tech. & Ther., 2002, 4:163-174).

The first cloning of a PPAR (PPARα) occurred in the course of the search for the molecular target of rodent hepatic peroxisome proliferating agents. Since then, numerous fatty acids and their derivatives, including a variety of eicosanoids and prostaglandins, have been shown to serve as ligands of the PPARs. Thus, these receptors may play a central role in the sensing of nutrient levels and in the modulation of their metabolism. In addition, PPARs are the primary targets of selected classes of synthetic compounds that have been used in the successful treatment of diabetes and dyslipidemia. As such, an understanding of the molecular and physiological characteristics of these receptors has become extremely important to the development and utilization of drugs used to treat metabolic disorders.

Kota, et al., Pharmacological Research, 2005, 51:85-94, provides a review of biological mechanisms involving PPARs that includes a discussion of the possibility of using PPAR modulators for treating a variety of conditions, including chronic inflammatory disorders such as atherosclerosis, arthritis and inflammatory bowel syndrome, retinal disorders associated with angiogenesis, increased fertility, and neurodegenerative diseases.

Yousef, et al., Journal of Biomedicine and Biotechnology, 2004(3): 156-166, discusses the anti-inflammatory effects of PPARα, PPARγ and PPARδ agonists, suggesting that PPAR agonists may have a role in treating neuronal diseases such as Alzheimer's disease, and autoimmune diseases such as inflammatory bowel disease and multiple sclerosis. A potential role for PPAR agonists in the treatment of Alzheimer's disease has been described in Combs, et al., Journal of Neuroscience 2000, 20(2):558, and such a role for PPAR agonists in Parkinson's disease is discussed in Breidert, et al., Journal of Neurochemistry, 2002, 82:615. A potential related function of PPAR agonists in treatment of Alzheimer's disease, that of regulation of the APP-processing enzyme BACE, has been discussed in Sastre, et al., Journal of Neuroscience, 2003, 23(30):9796. These studies collectively indicate PPAR agonists may provide advantages in treating a variety of neurodegenerative diseases by acting through complementary mechanisms.

Discussion of the anti-inflammatory effects of PPAR agonists is also available in Feinstein, Drug Discovery Today: Therapeutic Strategies, 2004, 1(1):29-34, in relation to multiple sclerosis and Alzheimer's disease; Patel, et al., Journal of Immunology, 2003, 170:2663-2669 in relation to chronic obstructive pulmonary disease and asthma (COPD); Lovett-Racke, et al., Journal of Immunology, 2004, 172:5790-5798 in relation to autoimmune disease; Malhotra, et al., Expert Opinions in Pharmacotherapy, 2005, 6(9):1455-1461, in relation to psoriasis; and Storer, et al., Journal of Neuroimmunology, 2005, 161:113-122, in relation to multiple sclerosis.

This wide range of roles for the PPARs that have been discovered suggest that PPARα, PPARγ and PPARδ may play a role in a wide range of events involving the vasculature, including atherosclerotic plaque formation and stability, thrombosis, vascular tone, angiogenesis, cancer, pregnancy, pulmonary disease, autoimmune disease, and neurological disorders.

Among the synthetic ligands identified for PPARs are thiazolidinediones (TZDs). These compounds were originally developed on the basis of their insulin-sensitizing effects in animal pharmacology studies. Subsequently, it was found that TZDs induced adipocyte differentiation and increased expression of adipocyte genes, including the adipocyte fatty acid-binding protein aP2. Independently, it was discovered that PPARγ interacted with a regulatory element of the aP2 gene that controlled its adipocyte-specific expression. On the basis of these seminal observations, experiments were performed that determined that TZDs were PPARγ ligands and agonists and demonstrate a definite correlation between their in vitro PPARγ activities and their in vivo insulin-sensitizing actions. (Bergen, et al., supra).

Several TZDs, including troglitazone, rosiglitazone, and pioglitazone, have insulin-sensitizing and anti-diabetic activity in humans with type 2 diabetes and impaired glucose tolerance. Farglitazar is a very potent non-TZD PPAR-γ-selective agonist that was recently shown to have anti-diabetic as well as lipid-altering efficacy in humans. In addition to these potent PPARγ ligands, a subset of the non-steroidal anti-inflammatory drugs (NSAIDs), including indomethacin, fenoprofen, and ibuprofen, have displayed weak PPARγ and PPARα activities. (Bergen, et al., supra).

The fibrates, amphipathic carboxylic acids that have been proven useful in the treatment of hypertriglyceridemia, are PPARα ligands. The prototypical member of this compound class, clofibrate, was developed prior to the identification of PPARs, using in vivo assays in rodents to assess lipid-lowering efficacy. (Bergen, et al., supra).

Fu et al., Nature, 2003, 425:9093, demonstrated that the PPARα binding compound, oleylethanolamide, produces satiety and reduces body weight gain in mice.

Clofibrate and fenofibrate have been shown to activate PPARα with a 10-fold selectivity over PPARγ. Bezafibrate acts as a pan-agonist that shows similar potency on all three PPAR isoforms. Wy-14643, the 2-arylthioacetic acid analogue of clofibrate, is a potent murine PPARα agonist as well as a weak PPAR-γ agonist. In humans, all of the fibrates must be used at high doses (200-1,200 mg/day) to achieve efficacious lipid-lowering activity.

TZDs and non-TZDs have also been identified that are dual PPAR-γ/α agonists. By virtue of the additional PPARα agonist activity, this class of compounds has potent lipid-altering efficacy in addition to anti-hyperglycemic activity in animal models of diabetes and lipid disorders. KRP-297 is an example of a TZD dual PPARγ/α agonist (Fajas, J. Biol. Chem., 1997, 272:18779-18789); furthermore, DRF-2725 and AZ-242 are non-TZD dual PPARγ/α agonists. (Lohray, et al., J. Med. Chem., 2001, 44:2675-2678; Cronet, et al., Structure (Camb.), 2001, 9:699-706).

In order to define the physiological role of PPARδ, efforts have been made to develop novel compounds that activate this receptor in a selective manner. Amongst the α-substituted carboxylic acids previously described, the potent PPARδ ligand L-165041 demonstrated approximately 30-fold agonist selectivity for this receptor over PPARγ, and it was inactive on murine PPARα (Liebowitz, et al., 2000, FEBS Lett., 473:333-336). This compound was found to increase high-density lipoprotein levels in rodents. It was also reported that GW501516 was a potent, highly-selective PPARδ agonist that produced beneficial changes in serum lipid parameters in obese, insulin-resistant rhesus monkeys. (Oliver et al., Proc. Natl. Acad. Sci., 2001, 98:5306-5311).

In addition to the compounds discussed above, certain thiazole derivatives active on PPARs have been described. (Cadilla, et al., Internat. Appl. PCT/US01/149320, Internat. Publ. WO 02/062774, incorporated herein by reference in its entirety.)

Some tricyclic-α-alkyloxyphenylpropionic acids have been described as dual PPARα/γ agonists in Sauerberg, et al., J. Med. Chem. 2002, 45:789-804.

A group of compounds that are stated to have equal activity on PPARα/γ/δ is described in Morgensen, et al., Bioorg. & Med. Chem. Lett., 2002, 13:257-260.

Oliver et al., describes a selective PPARδ agonist that promotes reverse cholesterol transport. (Oliver, et al., supra)

Yamamoto et al., U.S. Pat. No. 3,489,767 describes “1-(phenylsulfonyl)-indolyl aliphatic acid derivatives” that are stated to have “antiphlogistic, analgesic and antipyretic actions.” (Col. 1, lines 16-19.)

Kato, et al., European patent application 94101551.3, Publication No. 0 610 793 A1, describes the use of 3-(5-methoxy-1-p-toluenesulfonylindol-3-yl)propionic acid (page 6) and 1-(2,3,6-triisopropylphenylsulfonyl)-indole-3-propionic acid (page 9) as intermediates in the synthesis of particular tetracyclic morpholine derivatives useful as analgesics.

### SUMMARY OF THE INVENTION

The present invention relates to compounds active on PPARs, which are useful for a variety of applications including, for example, therapeutic and/or prophylactic methods involving modulation of at least one of PPARα, PPARδ, and PPARγ. Included are compounds that have pan-activity across the PPAR family (i.e., PPARα, PPARδ, and PPARγ), as well as compounds that have significant specificity (at least 5-, 10-, 20-, 50-, or 100-fold greater activity) on a single PPAR, or on two of the three PPARs.

In one aspect, the invention provides compounds of Formula I as follows:

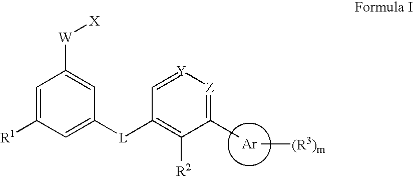

all salts, prodrugs, tautomers and isomers thereof,
wherein:
W is selected from the group consisting of a covalent bond, —NR⁴(CR⁵R⁶)₁₋₂—, —O—(CR⁵R⁶)₁₋₂—, —S—(CR⁵R⁶)₁₋₂—, —CHR⁶—, —(CR⁵R⁶)₂₋₃—, and —CR⁷═CR⁸—;
X is selected from the group consisting of —C(O)OR⁹, —C(O)NR¹⁰R¹¹, and a carboxylic acid isostere;
Y is CH or N;
Z is CH or N;
L is —NR⁴S(O)₂—, —S—, —S(O)—, —S(O)₂— or —O—;
Ar is aryl or heteroaryl;
R¹ is hydrogen, fluoro, chloro, methoxy, fluoro substituted methoxy, C₃₋₅ cycloalkyl, C₁₋₃ alkyl, or C₁₋₃ alkyl substituted with one or more fluoro, methoxy, or fluoro substituted methoxy;
R² is hydrogen, fluoro, chloro, C₁₋₃ alkyl or fluoro substituted C₁₋₃ alkyl;
R³ at each occurrence is independently selected from the group consisting of halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —NO₂, —CN, —OR¹², —NR¹²R¹³, —C(V)NR¹²R¹³, —C(V)R¹⁴, —S(O)₂NR¹²R¹³, —S(O)R¹⁴, —OC(V)R¹⁴, —C(V)OR¹², —C(NH)NR¹⁵R¹⁶, —NR¹²C(V)R¹⁴, —NR¹²S(O)₂R¹⁴, —NR¹²C(V)NR¹²R¹³, and —NR¹²S(O)₂NR¹²R¹³;
R⁴ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, provided, however, that any substitution on the alkyl carbon bound to the N of NR⁴ is fluoro, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁵ and R⁶ at each occurrence are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio;
R⁷ and R⁸ are independently hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio;
R⁹ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁹ is lower alkyl, any substitution on the alkyl carbon bound to the O of OR⁹ is fluoro;
R¹⁰ and R¹¹ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁰ and/or R¹¹ is lower alkyl, any substitution on the alkyl carbon bound to the N of NR¹⁰R¹¹ is fluoro; or
R¹⁰ and R¹¹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl fluoro substituted lower alkyl, lower alkoxy, fluoro substituted lower alkoxy, lower alkylthio, and fluoro substituted lower alkylthio;
R¹², R¹³, R¹⁵, and R¹⁶ at each occurrence are independently selected from the group consisting of hydrogen, optionally substituted lower alkyl, optionally substituted C₃₋₆ alkenyl, provided, however, that when R¹², R¹³, R¹⁵, or R¹⁶ is optionally substituted C₃₋₆ alkenyl, no alkene carbon thereof is bound to the O of any OR¹² or N of any NR¹², NR¹³, NR¹⁵, or NR¹⁶; optionally substituted C₃₋₆ alkynyl, provided, however, that when R¹², R¹³, R¹⁵, or R¹⁶ is optionally substituted C₃₋₆ alkynyl, no alkyne carbon thereof is the O of any OR¹² or N of any NR¹², NR¹³, NR¹⁵ or NR¹⁶; optionally substituted cycloalkyl, optionally substituted heterocycloalkyl, optionally substituted aryl, and optionally substituted heteroaryl, or
R¹⁵ and R¹⁶ combine with the nitrogen to which they are attached to form a 5-7 membered optionally substituted heterocycloalkyl or a 5 or 7 membered optionally substituted nitrogen containing heteroaryl;
R¹⁴ at each occurrence is independently selected from the group consisting of optionally substituted lower alkyl, optionally substituted C₃₋₆ alkenyl, provided, however, that when R¹⁴ is optionally substituted C₃₋₆ alkenyl, no alkene carbon thereof is bound to the S of any S(O)R¹⁴ or the C of any C(Z)R¹⁴; optionally substituted C₃₋₆ alkynyl, provided, however, that when R¹⁴ is optionally substituted C₃₋₆ alkynyl, no alkyne carbon thereof is bound to the S of any S(O)ₙR¹⁴ or the C of any C(Z)R¹⁴; optionally substituted cycloalkyl, optionally substituted heterocycloalkyl, optionally substituted aryl, and optionally substituted heteroaryl;
V is O or S;
n is 0, 1 or 2; and
m is 0, 1, 2, 3, 4, or 5, provided, however, the compound is not

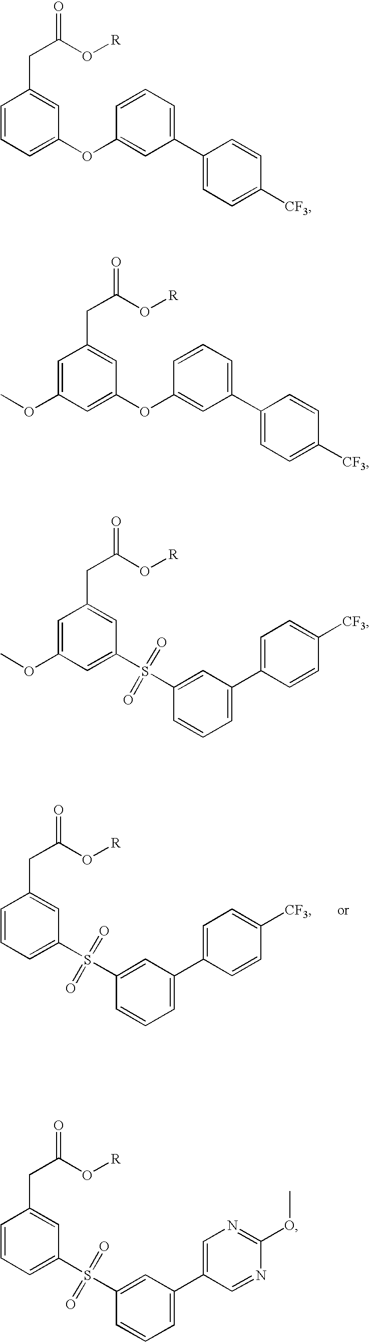

wherein R is H, methyl or ethyl

In some embodiments of compounds of Formula I, W is —O—CR⁵R⁶—, —CHR⁶—, or —(CR⁵R⁶)₂—, preferably —CHR⁶—, preferably —CH₂—. In some embodiments, X is —C(O)OR⁹, preferably —C(O)OH. In some embodiments, L is —O—. In some embodiments, L is —S—. In some embodiments. L is —S(O)—. In some embodiments, L is —S(O)₂—. In some embodiments, L is —NR⁴S(O)₂—. In some embodiments, W is —O—CR⁵R⁶—, —CHR⁶—, or —(CR⁵R⁶)₂—, preferably —CHR⁶—, preferably —CH₂— and X is —C(O)OR⁹, preferably —C(O)OH. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and L is —O—. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and L is —S—. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and L is —S(O)—. In some embodiments, W is —O—CR⁵R⁶—, —CHR⁶—, or —(CR⁵R⁶)₂—, preferably —CHR⁶—, preferably —CH₂—, X is —C(OR⁹, preferably —C(O)OH, and L is —S(O)₂—. In some embodiments, W is —O—CR⁵R⁶—, —CHR⁶—, or —(CR⁵R⁶)₂—, preferably —CHR⁶—, preferably —CH₂—X is —C(O)OR⁹, preferably —C(O)OH, and L is —NR⁴S(O)₂—. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, L is —O—, and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, L is —S—, and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl. In some embodiments, W is —O—CR⁵R⁶—, —CHR⁶—, or —(CHR⁶)₂—, preferably —CHR⁶—, preferably —CH₂—, X is —C(O)OR⁹, preferably —C(O)OH, L is —S(O)—, and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, is —S(O)₂—, and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, L is —NR⁴S(O)₂—, and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl.

In some embodiments of compounds of Formula I, one of Y and Z is N and the other of Y and Z is CH. In some embodiments, Y is N and Z is CH. In some embodiments, Y is N, Z is CH, and R² is hydrogen. In some embodiments, Y is CH and Z is N. In some embodiments, Y is CH, Z is N, and R² is hydrogen. In some embodiments, both Y and Z are CH. In some embodiments, both Y and Z are CH and R² is hydrogen. In some embodiments, both Y and Z are CH and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl. In some embodiments, both Y and Z are CH, R² is hydrogen and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl. In some embodiments, both Y and Z are CH, W is —O—CR⁵R⁶—, —CHR⁶—, or —(CR⁵R⁶)₂—, preferably —CHR⁶, preferably —CH₂— and X is —C(O)OR⁹, preferably —C(O)OH. In some embodiments, both Y and Z are CH, W is —O—CR⁵R⁶—, —CHR⁶—, or —(CR⁵R⁶)₂—, preferably —CHR⁶—, preferably —CH₂—, X is C(O)OR⁹, preferably —C(O)OH, and Ar is phenyl or monocyclic heteroaryl, preferably phenyl, pyridinyl, pyrimidinyl, pyrazolyl, imidazolyl, thiazolyl, isothiazolyl, oxazolyl, or isoxazolyl.

In some embodiments of compounds of Formula I, Ar is phenyl or monocyclic heteroaryl. In some embodiments, Ar is phenyl, pyridinyl, pyrimidinyl, pyrazolyl, imidazolyl, thiazolyl, isothiazolyl, oxazolyl, or isoxazolyl.

In some embodiments, compounds of Formula I have the structure according to the following sub-generic structure Formula Ia:

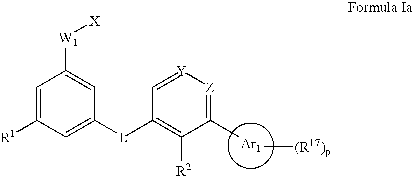

all salts, prodrugs, tautomers and isomers thereof,
wherein:
W₁ is —O—CR⁵R⁶—, —CHR⁶—, or —(CR⁵R⁶)₂—;
R⁵, R⁶, X, Y, Z, L, R¹ and R² are as defined for Formula I;
Ar₁ is phenyl or monocyclic heteroaryl;
R¹⁷ at each occurrence is independently selected from the group consisting of —OH, —NH₂, —NO₂, —CN, —C(O)OH, —S(O)₂NH₂, —C(O)NH₂, —OR¹⁸, —SR¹⁸, —NR¹⁹R¹⁸, —NR¹⁹C(O)R¹⁸, —NR¹⁹S(O)₂R¹⁸, —S(O)₂R¹⁸, —C(O)R¹⁸, —C(O)OR¹⁸, —C(O)NR¹⁹R¹⁸, —S(O)₂NR¹⁹R¹⁸, halogen, lower alkyl, cycloalkyl, heterocycloalkyl, aryl and heteroaryl, wherein lower alkyl is optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, cycloalkyl, heterocycloalkyl, aryl, and heteroaryl, wherein cycloalkyl, heterocycloalkyl, aryl, and heteroaryl as R¹⁷, or as substituents of lower alkyl, are optionally substituted with one or more substituents selected from the group consisting of —OH, —NH₂, —NO₂, —CN, —C(O)OH, —S(O)₂NH₂, —C(O)NH₂, —OR²⁰, —SR²⁰, —NR¹⁹R²⁰, —NR¹⁹C(O)R²⁰, —NR¹⁹S(O)₂R²⁰, —S(O)₂R²⁰, —C(O)R²⁰, —C(O)OR²⁰, —C(O)NR¹⁹R²⁰, —S(O)₂NR¹⁹R²⁰, halogen, lower alkyl, fluoro substituted lower alkyl, and cycloalkylamino;
R¹⁸ at each occurrence is independently selected from the group consisting of lower alkyl, cycloalkyl, heterocycloalkyl, aryl and heteroaryl, wherein lower alkyl is optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, cycloalkyl, heterocycloalkyl, aryl, and heteroaryl, provided, however, that any substitution of the alkyl carbon bound to O, S, or N of any OR¹⁸, SR¹⁸, or NR¹⁸ is fluoro, cycloalkyl, heterocycloalkyl, aryl or heteroaryl, wherein cycloalkyl, heterocycloalkyl, aryl, and heteroaryl as R¹⁸ or as substituents of lower alkyl are optionally substituted with one or more substituents selected from the group consisting of —OH, —NH₂, —NO₂, —CN, —C(O)OH, —S(O)₂NH₂, —C(O)NH₂, —OR²⁰, —SR²⁰, —NR¹⁹R²⁰, —NR¹⁹C(O)R²⁰, —NR¹⁹S(O)₂R²⁰, —S(O)₂R²⁰, —C(O)R²⁰, —C(O)OR²⁰, —C(O)NR¹⁹R²⁰, —S(O)₂NR¹⁹R²⁰, halogen, lower alkyl, fluoro substituted lower alkyl, and cycloalkylamino;
R¹⁹ at each occurrence is independently hydrogen or lower alkyl, wherein lower alkyl is optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino;
R²⁰ at each occurrence is independently selected from the group consisting of lower alkyl, heterocycloalkyl and heteroaryl, wherein lower alkyl is optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl carbon bound to O, S, or N of any OR²⁰, SR²⁰, or NR²⁰ is fluoro; and
p is 0, 1, 2, or 3.

In some embodiments, compounds of Formula I have the structure according to the following sub-generic structure Formula Ib:

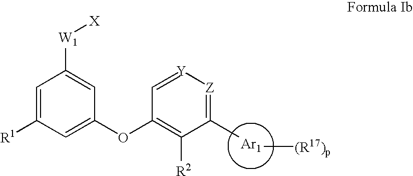

all salts, prodrugs, tautomers and isomers thereof,
wherein:
X, Y, Z, R¹ and R² are as defined for Formula I; and
W₁, Ar₁, p and R¹⁷ are as defined for Formula Ia.

In some embodiments, compounds of Formula I have the structure according to the g sub-generic structure Formula Ic:

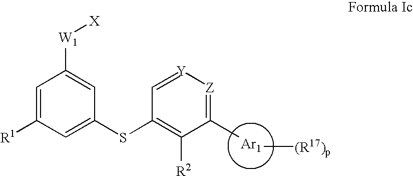

all salts, prodrugs, tautomers and isomers thereof,
wherein:
X, Y, Z, R¹ and R² are as defined for Formula I; and
W₁, Ar₁, p and R¹⁷ are as defined for Formula Ia.

In some embodiments, compounds of Formula I have the structure according to the following sub-generic structure Formula Id:

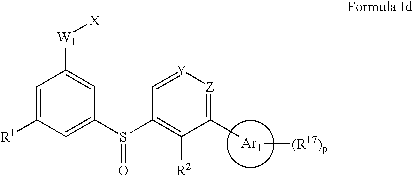

all salts, prodrugs, tautomers and isomers thereof,
wherein:
X, Y, Z, R¹ and R² are as defined for Formula I; and
W₁, Ar₁, p and R¹⁷ are as defined for Formula Ia.

In some embodiments, compounds of Formula I have the structure according to the following sub-generic structure Formula Ie:

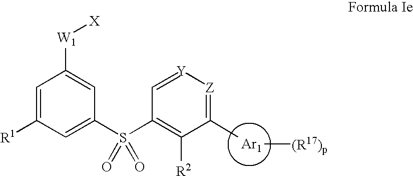

all salts, prodrugs, tautomers and isomers thereof,
wherein:
X, Y, Z, R¹ and R² are as defined for Formula I; and
W₁, Ar₁, p and R¹⁷ are as defined for Formula Ia.

In some embodiments, compounds of Formula I have the structure according to the following sub-generic structure Formula If:

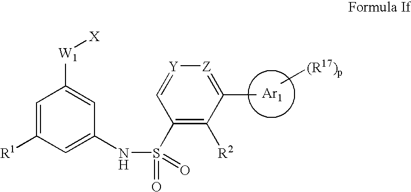

all salts, prodrugs, tautomers and isomers thereof,
wherein:
X, Y, Z, R¹ and R² are as defined for Formula I; and
W₁, Ar₁, p and R¹⁷ are as defined for Formula Ia.

In some embodiments of compounds of Formula Ia, Ib, Ic, Id, Ie, or If, further to any of the embodiments contemplated herein of Formula Ia, Ib, Ic, Id, Ie, or If, W is —CHR⁶—, preferably —CH₂—. In some embodiments, X is —C(O)OR⁹, preferably —C(O)OH. In some embodiments, W is —CHR⁶—, preferably —CH₂— and X is —C(O)OR⁹, preferably —C(O)OH. In some embodiments, W is —CHR⁶—, preferably —CH₂—, X is —C(O)OR⁹, preferably —C(O)OH, and Ar is phenyl, pyridinyl, pyrimidinyl, pyrazolyl, imidazolyl, thiazolyl, isothiazolyl, oxazolyl, or isoxazolyl.

In some embodiments of compounds of Formula Ia, Ib, Ic, Id, Ie, or If, further to any of the embodiments contemplated herein of Formula Ia, Ib, Ic, Id, Ie, or If, one of Y and Z is N and the other of Y and Z is CH. In some embodiments, Y is N and Z is CH. In some embodiments, Y is N, Z is CH, and R² is hydrogen. In some embodiments. Y is CH and Z is N. In some embodiments, Y is CH, Z is N, and R² is hydrogen. In some embodiments, both Y and Z are CH. In some embodiments, both Y and Z are CH and R² is hydrogen. In some embodiments, both Y and Z are CH and Ar is phenyl, pyridinyl, pyrimidinyl, pyrazolyl, imidazolyl, thiazolyl, isothiazolyl, oxazolyl, or isoxazolyl. In some embodiments, both Y and Z are CH, R² is hydrogen and Ar₁ is phenyl, pyridinyl, pyrimidinyl, pyrazolyl, imidazolyl, thiazolyl, isothiazolyl, oxazolyl, or isoxazolyl. In some embodiments, both Y and Z are CH, W is —CR⁵R⁶—, preferably —CH₂— and X is —C(O)OR⁹, preferably —C(O)OH. In some embodiments, both Y and Z are CH, W is —CHR⁶—, preferably —CH₂—, X is —C(O)OR⁹, preferably —C(O)OH, and Ar₁ is phenyl, pyridinyl, pyrimidinyl, pyrazolyl, imidazolyl, thiazolyl, isothiazolyl, oxazolyl, or isoxazolyl.

In some embodiments of compounds of Formula Ia, Ib, Ic, Id, Ie, or If, further to any of the embodiments contemplated herein of Formula Ia, Ib, Ic, Id, Ie, or If, Ar₁ is phenyl, pyridinyl, pyrimidinyl, pyrazolyl, imidazolyl, thiazolyl, isothiazolyl, oxazolyl, or isoxazolyl. In some embodiments, Ar₁ is phenyl, pyridinyl, pyrimidinyl, pyrazolyl, imidazolyl, thiazolyl, isothiazolyl, oxazolyl, or isoxazolyl and R⁵ and R⁶ are both hydrogen at all occurrences. In some embodiments, Ar₁ is phenyl, pyridinyl, pyrimidinyl, pyrazolyl, imidazolyl, thiazolyl, isothiazolyl, oxazolyl, or isoxazolyl, R⁵ and R⁶ are both hydrogen at all occurrences, and X is —C(O)OR⁹, preferably —C(O)OH. In some embodiments, Ar₁ is phenyl, pyridinyl, pyrimidinyl, pyrazolyl, imidazolyl, thiazolyl, isothiazolyl, oxazolyl, or isoxazolyl, W is —CH₂—, X is —C(O)OR⁹, preferably —C(O)OH, Y and Z are CH, and R² is hydrogen.

In some embodiments of compounds of Formula Ia, Ib, Ic, Id, Ie, or If, further to any of the embodiments contemplated herein of Formula Ia, Ib, Ic, Id, Ie, or If, R¹⁷ is selected from the group consisting of halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio.

In some embodiments, compounds of Formula I have the structure selected from the following sub-generic structures Formula Ig, Formula Ih, Formula Ii, Formula Ij, Formula Ik, Formula Im, Formula In, and Formula Io:

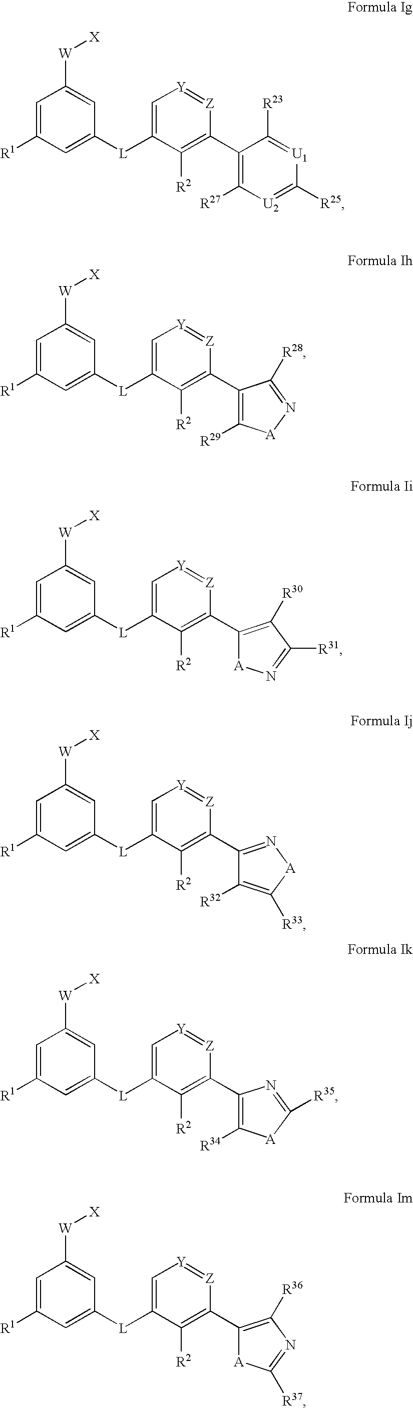

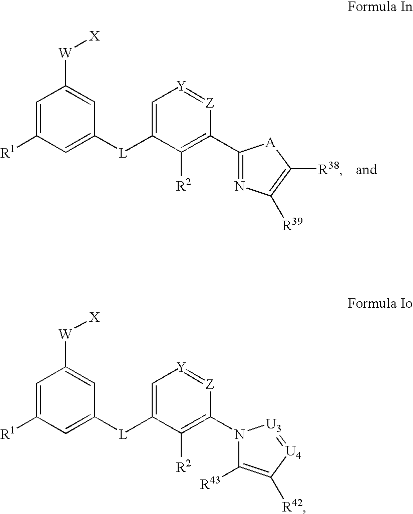

all salts, prodrugs, tautomers and isomers thereof,
wherein:
W, X, Y, Z, L, R¹, and R² are as defined for Formula I;
U₁ is Nor CR²⁴;
U₂ is N or CR²⁶;
U₃ is N or CR⁴⁰;
U₄ is N or CR⁴¹;
A is O, S, or NR⁴⁴;
R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², and R⁴³, are independently hydrogen or R³ as defined in Formula I: and
R⁴⁴ is selected from the group consisting of hydrogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —C(V)NR¹²R¹³, —C(V)R¹⁴, —S(O)₂NR¹²R¹³, —S(O)₂R¹⁴, C(V)OR¹², and —C(NH)NR¹⁵R¹⁶, wherein V, R¹², R¹³, R¹⁴, R¹⁵ and R¹⁶ are as defined for Formula I.

In some embodiments of compounds of Formulae Ig-Io, R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², and R⁴³, are independently hydrogen or R¹⁷, wherein R¹⁷ is as defined in paragraph [0032], preferably wherein R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R⁴¹, R⁴², and R⁴³, are selected from the group consisting of hydrogen, halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio, and R⁴⁴ is selected from the group consisting of hydrogen, —C(O)OH, —S(O)₂NH₂, —C(O)NH₂, —S(O)₂R¹⁸, —C(O)R¹⁸, —C(O)OR¹⁸, —C(O)NR¹⁹R¹⁸, —S(O)₂NR¹⁹R¹⁸, lower alkyl, cycloalkyl, heterocycloalkyl, aryl and heteroaryl, wherein lower alkyl is optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, cycloalkyl, heterocycloalkyl, aryl, and heteroaryl, wherein cycloalkyl, heterocycloalkyl, aryl, and heteroaryl as R⁴⁴, or as substituents of lower alkyl, are optionally substituted with one or more substituents selected from the group consisting of —OH, —NH₂, —NO₂, —CN, —C(O)OH, —S(O)₂NH₂, —C(O)NH₂, —OR²⁰, —SR²⁰, —NR¹⁹R²⁰, —NR¹⁹C(O)R²⁰, —NR¹⁹S(O)₂R²⁰, —S(O)₂R²⁰, —C(O)R²⁰, —C(O)OR²⁰, —C(O)NR¹⁹R²⁰, —S(O)₂NR¹⁹R²⁰, halogen, lower alkyl, fluoro substituted lower alkyl, and cycloalkylamino, wherein R¹⁸, R¹⁹, and R²⁰ are as defined in paragraph [0032], preferably R⁴⁴ is hydrogen or lower alkyl optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino.

In some embodiments of compounds of Formula Ig, R²⁶ and R²⁷ are hydrogen. In some embodiments, R²⁷ is hydrogen and U₁ and U₂ are N. In some embodiments, R²⁷ is hydrogen. U₂ is CH and U₁ is N. In some embodiments, R²⁷ is hydrogen, U₂ is CH and U₁ is CR²⁴. In some embodiments, R²⁷ is hydrogen, U₂ is N or CH, U₁ is N or CR²⁴, and R²³, R²⁴ and R²⁵ are independently hydrogen or R³, preferably hydrogen or R¹⁷, more preferably R²³, R²⁴ and R²⁵ are independently selected from the group consisting of hydrogen, halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio.

In some embodiments of compounds of Formula Ig, R²³ and R²⁷ are H, U₂ is CH, U₁ is CH, and R²⁵ is independently hydrogen or R³, preferably hydrogen or R¹⁷, preferably R¹⁷, more preferably R²⁵ is independently selected from the group consisting of halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio.

In some embodiments of compounds of Formula Ig, R²³, R²⁵, and R²⁷ are H, U₂ is CH, U₁ is CR²⁴, and R²⁴ is independently hydrogen or R³, preferably hydrogen or R¹⁷, preferably R¹⁷, more preferably R²⁴ is independently selected from the group consisting of halogen, —OH, —NH₂, —NO₂, —CN, lower alkyl, lower alkoxy, lower alkylthio, mono-alkylamino, di-alkylamino, and —NR²¹R²² wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio.

In some embodiments of compounds of Formula Ig, R²⁵ and R²⁷ are H, U₂ is CH, U₁ is CH and R²³ is independently hydrogen or R³, preferably hydrogen or R¹⁷, preferably R¹⁷, more preferably R²³ is independently selected from the group consisting of halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio.

In some embodiments of compounds of Formula Ig, R²⁷ is H, U₂ is CH, U₁ is CH and R²³ and R²⁵ are independently hydrogen or R³, preferably hydrogen or R¹⁷, preferably R¹⁷, more preferably R²³ and R²⁵ are independently selected from the group consisting of halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio.

In some embodiments of compounds of Formula Ig, R²⁷ and R²³ are H, U₂ is CH, UL is CR²⁴ and R²⁴ and R²⁵ are independently hydrogen or R³, preferably hydrogen or R¹⁷, preferably R¹⁷ more preferably R²⁴ and R²⁵ are independently selected from the group consisting of halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl fluoro substituted lower alkyl, lower alkoxy, fluoro substituted lower alkoxy, lower alkylthio, and fluoro substituted lower alkylthio.

In some embodiments of compounds of Formula Ig, R²⁷ and R²⁵ are H, U₂ is CH, U₁ is CR²⁴ and R²³ and R²⁴ are independently hydrogen or R³, preferably hydrogen or R¹⁷, preferably R¹⁷, more preferably R²³ and R²⁴ are independently selected from the group consisting of halogen, —OH, —NH₂, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio.

In some embodiments of compounds of Formula Ih, A is NR⁴⁴. In some embodiments, A is NR⁴⁴, R⁴⁴ is hydrogen or lower alkyl optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, and R²⁸ and R²⁹ are independently selected from the group consisting of hydrogen, halogen, —OH, —NIH2, —NO₂, —CN, lower alkyl, lower alkoxy, lower alkylthio, mono-alkylamino, di-alkylamino, and —NR²¹R²², wherein lower alkyl and the alkyl chain(s) of lower alkoxy, lower alkylthio, mono-alkylamino or di-alkylamino are optionally substituted with one or more, preferably 1, 2, or 3 substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, wherein R²¹ and R²² combine with the nitrogen to which they are attached to form a 5-7 membered heterocycloalkyl or 5-7 membered heterocycloalkyl substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio, preferably R²⁸ and R²⁹ are both hydrogen.

In some embodiments of compounds of Formula Ig-Io, further to any of the embodiments contemplated herein of Formula Ig-Io, W is —O—CR⁵R⁶—, —CHR⁶—, or —(CR⁵R⁶)₂—, preferably —CHR⁶—, preferably —CH₂—. In some embodiments, X is —C(O)OR⁹, preferably —C(O)OH. In some embodiments, L is —O—. In some embodiments, L is —S—. In some embodiments, L is —S(O)—. In some embodiments, L is —S(O)₂—. In some embodiments, L is —NR⁴S(O)₂—. In some embodiments, W is —O—CR⁵R⁶—, —CHR⁶—, or —(CR⁵R⁶)₂—, preferably —CHR⁶—, preferably —CH₂— and X is —C(O)OR⁹, preferably —C(O)OH. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and L is —O—. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and L is —S—. In some embodiments, W is —O—CR⁵R⁶—, —CHR⁶—, or —(CR⁵R⁶)₂—, preferably —CHR⁶—, preferably —CH₁—, X is —C(O)OR⁹, preferably —C(O)OH, and L is —S(O)—. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and L is —S(O)₂—. In some embodiments, W is —O—CR⁵R⁶—, —CHR⁶—, or —(CR⁵R⁶)₂—, preferably —CHR⁶—, preferably —CH₂—, X is —C(O)OR⁹, preferably —C(O)OH, and L is —NR⁴S(O)₂—.

In some embodiments of compounds of Formula Ig-Io, further to any of the embodiments contemplated herein of Formula Ig-Io, one of Y and Z is N and the other of Y and Z is CH. In some embodiments, Y is N and Z is CH. In some embodiments, Y is N, Z is CH, and R² is hydrogen. In some embodiments, Y is CH and Z is N. In some embodiments, Y is CH Z is N, and R² is hydrogen. In some embodiments, both Y and Z are CH. In some embodiments, both Y and Z are CH and R² is hydrogen.

In some embodiments, compounds of Formula I have the following sub-generic structure Formula Ip:

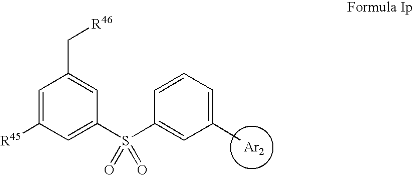

all salts, prodrugs, tautomers and isomers thereof,
wherein:
R⁴⁵ is hydrogen, chloro, methyl, or methoxy;
R⁴⁶ is selected from the group consisting of —C(O)OR⁴⁷, —C(O)NR⁴⁸R⁴⁹, and a carboxylic acid isostere;
R⁴⁷ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁴⁷ is lower alkyl, any substitution on the alkyl carbon bound to the O of OR⁴⁷ is fluoro;
R⁴⁸ and R⁴⁹ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁴⁸ and/or R⁴⁹ is lower alkyl, any substitution on the alkyl carbon bound to the N of NR⁴⁸R⁴⁹ is fluoro; or
R⁴⁸ and R⁴⁹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
Ar₂ is selected from the group consisting of:

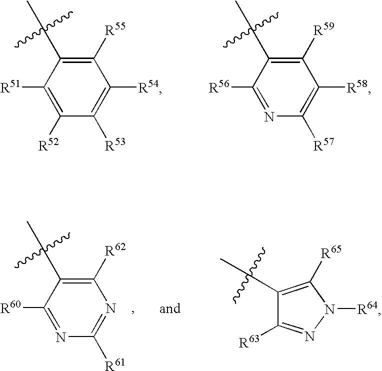

wherein

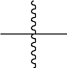

indicates the point of attachment of Ar₂ to the ring of Formula Ip;
R⁵¹, R⁵², R⁵³, R⁵⁴, R⁵⁵, R⁵⁸ and R⁵⁹ are independently selected from the group consisting of hydrogen, fluoro, chloro, C₁₋₃ alkyl, fluoro substituted C₁₋₃ alkyl, C₁₋₃ alkoxy, fluoro substituted C₁₋₃ alkoxy, and benzyloxy;
R⁵⁶, R⁵⁷, R⁶³ and R⁶⁵ are independently selected from the group consisting of hydrogen, fluoro, C₁₋₃ alkyl, fluoro substituted C₁₋₃ alkyl C₁₋₃ alkoxy, fluoro substituted C₁₋₃ alkoxy, and benzyloxy;
R⁶⁰, R⁶¹ and R⁶² are independently selected from the group consisting of hydrogen, C₁₋₃ alkyl, fluoro substituted C₁₋₃ alkyl, C₁₋₃ alkoxy, fluoro substituted C₁₋₃ alkoxy, and benzyloxy; and
R⁶⁴ is lower alkyl or fluoro substituted lower alkyl.

In some embodiments of compounds of Formula Ip, Ar₂ is

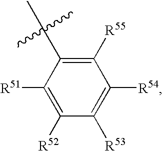

and R⁵¹, R⁵², R⁵³, R⁵⁴, and R⁵⁵ are independently selected from the group consisting of hydrogen, fluoro, chloro, methyl, trifluoromethyl, methoxy, trifluoromethoxy, ethoxy, and benzyloxy. In some embodiments, three of R⁵¹, R⁵², R⁵³, R⁵⁴, and R⁵⁵ are hydrogen and the others of R⁵¹, R⁵², R⁵³, R⁵⁴, and R⁵⁵ are independently selected from the group consisting of hydrogen, fluoro, chloro, methyl, trifluoromethyl, methoxy, trifluoromethoxy, ethoxy, and benzyloxy.

In some embodiments of compounds of Formula Ip, Ar₂ is

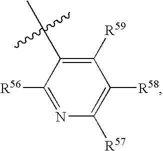

R⁵⁶, and R⁵⁷ are independently selected from the group consisting of hydrogen, fluoro, methyl, trifluoromethyl, methoxy, trifluoromethoxy, ethoxy, and benzyloxy, and R⁵⁸ and R⁵⁹ are independently selected from the group consisting of hydrogen, fluoro, chloro, methyl, trifluoromethyl, methoxy, trifluoromethoxy, ethoxy, and benzyloxy. In some embodiments R⁵⁶R⁵⁷, R⁵⁸, and R⁵⁹ are independently selected from the group consisting of hydrogen and methoxy.

In some embodiments of compounds of Formula Ip, Ar₂ is

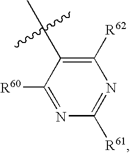

and R⁶⁰, R⁶¹, and R⁶² are independently selected from the group consisting of hydrogen, methyl, trifluoromethyl, methoxy, trifluoromethoxy, ethoxy, and benzyloxy. In some embodiments R⁶⁰, R⁶¹, and R⁶² are independently selected from the group consisting of hydrogen and methoxy.

In some embodiments of compounds of Formula Ip, Ar₂ is

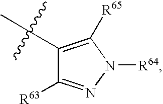

R⁶³ and R⁶⁵ are independently selected from the group consisting of hydrogen, fluoro, methyl, trifluoromethyl, methoxy, trifluoromethoxy, ethoxy, and benzyloxy, and R⁶⁴ is lower alkyl. In some embodiments, R⁶³ and R⁶⁵ are hydrogen and R⁶⁴ is lower alkyl.

In one embodiment of compounds of Formula I, the compound is selected from the group consisting of:
[3-(3′-Chloro-biphenyl-3-sulfonyl)-5-methoxy-phenyl]-acetic acid (P-0015),
[3-(4′-Chloro-biphenyl-3-sulfonyl)-5-methoxy-phenyl]-acetic acid (P-0016),
[3-Methoxy-5-(4′-methoxy-biphenyl-3-sulfonyl)-phenyl]-acetic acid (P-0017),
[3-(4′-Fluoro-biphenyl-3-sulfonyl)-5-methoxy-phenyl]-acetic acid (P-0018),
[3-(3′-Chloro-4′-fluoro-biphenyl-3-sulfonyl)-5-methoxy-phenyl]-acetic acid (P-0019),
[3-(4′-Ethoxy-biphenyl-3-sulfonyl)-5-methoxy-phenyl]-acetic acid (P-0020),
[3-(3′-Fluoro-biphenyl-3-sulfonyl)-5-methoxy-phenyl]-acetic acid (P-0021),
[3-Methoxy-5-(3′-trifluoromethoxy-biphenyl-3-sulfonyl)-phenyl]-acetic acid (P-0022),
[3-Methoxy-5-(4′-trifluoromethoxy-biphenyl-3-sulfonyl)-phenyl]-acetic acid (P-0023),
[3-Methoxy-5-(3′-trifluoromethyl-biphenyl-3-sulfonyl)-phenyl]-acetic acid (P-0024),
[3-(3′-Fluoro-4′-methyl-biphenyl-3-sulfonyl)-5-methoxy-phenyl]-acetic acid (P-0025),
[3-(3′-Fluoro-4′-met oxy-biphenyl-3-sulfonyl)-5-methoxy-phenyl]-acetic acid (P-0026),
[3-(2′-Fluoro-biphenyl-3-sulfonyl)-5-methoxy-phenyl]-acetic acid (P-0062),
[3-(2′-Fluoro-4′-methoxy-biphenyl-3-sulfonyl)-5-methoxy-phenyl]-acetic acid (P-0063),
[3-(Biphenyl-3-sulfonyl)-5-methoxy-phenyl]-acetic acid (P-0086),
[3-(2′,3′-Difluoro-biphenyl-3-sulfonyl)-5-methoxy-phenyl]-acetic acid (P-0087),
[3-(4′-Chloro-2′-methyl-biphenyl-3-sulfonyl)-5-methoxy-phenyl]-acetic acid (P-0088),
[3-(2′-Chloro-4′-trifluoromethyl-biphenyl-3-sulfonyl)-5-methoxy-phenyl]-acetic acid (P-0091),
[3-(2′-Fluoro-4′-trifluoromethyl-biphenyl-3-sulfonyl)-5-methoxy-phenyl]-acetic acid (P-0092), and all salts, prodrugs, tautomers, and isomers thereof.

In one embodiment of compounds of Formula I, the compound is selected from the group consisting of:
[3-(3′-Chloro-biphenyl-3-sulfonyl)-phenyl]-acetic acid (P-0027),
[3-(4′-Chloro-biphenyl-3-sulfonyl)-phenyl]-acetic acid (P-0028),
[3-(4′-Methoxy-biphenyl-3-sulfonyl)-phenyl]-acetic acid (P-0029),
[3-(4′-Fluoro-biphenyl-3-sulfonyl)-phenyl]-acetic acid (P-0030),
[3-(4′-Ethoxy-biphenyl-3-sulfonyl)-phenyl]-acetic acid (P-0031),
[3-(3′-Fluoro-biphenyl-3-sulfonyl)-phenyl]-acetic acid (P-0032),
[3-(3′-Trifluoromethoxy-biphenyl-3-sulfonyl)-phenyl]-acetic acid (P-0033),
[3-(4′-Trifluoromethoxy-biphenyl-3-sulfonyl)-phenyl]-acetic acid (P-0034),
[3-(3′-Trifluoromethyl-biphenyl-3-sulfonyl)-phenyl]-acetic acid (P-0035),
[3-(3′-Fluoro-4′-methyl-biphenyl-3-sulfonyl)-phenyl]-acetic acid (P-0036),
[3-(3′-Fluoro-4′-methoxy-biphenyl-3-sulfonyl)-phenyl]-acetic acid (P-0037),
[3-(2′-Fluoro-biphenyl-3-sulfonyl)-phenyl]-acetic acid (P-0064),
[3-(4′-Benzyloxy-2′-fluoro-biphenyl-3-sulfonyl)-phenyl]-acetic acid (P-0065),
[3-(2′-Fluoro-4′-methoxy-biphenyl-3-sulfonyl)-phenyl]-acetic acid (P-0066),
[3-(4′-Chloro-2′-fluoro-biphenyl-3-sulfonyl)-phenyl]-acetic acid (P-0067),
[3-(Biphenyl-3-sulfonyl)-phenyl]-acetic acid (P-0080),
[3-(4′-Fluoro-2′-methyl-biphenyl-3-sulfonyl)-phenyl]-acetic acid (P-0081),
[3-(2′,3′-Difluoro-biphenyl-3-sulfonyl)-phenyl]-acetic acid (P-0082),
[3-(4′-Chloro-2′-methyl-biphenyl-3-sulfonyl)-phenyl]-acetic acid (P-0083),
[3-(2′-Chloro-4′-ethoxy-biphenyl-3-sulfonyl)-phenyl]-acetic acid (P-0084),
[3-(4′-Ethoxy-2′-methyl-biphenyl-3-sulfonyl)-phenyl]-acetic acid (P-0085),
[3-(2′-Fluoro-4′-trifluoromethyl-biphenyl-3-sulfonyl)-phenyl]-acetic acid (P-0093),
[3-(2′-Chloro-4′-trifluoromethyl-biphenyl-3-sulfonyl)-phenyl]-acetic acid (P-0094), and
   all salts, prodrugs, tautomers, and isomers thereof.

In one embodiment of compounds of Formula I, the compound is selected from the group consisting of:
{3-Methoxy-5-[3-(6-methoxy-pyridin-3-yl)-benzenesulfonyl]-phenyl}-acetic acid (P-0001),
{3-Methoxy-5-[3-(2-methoxy-pyrimidin-5-yl)-benzenesulfonyl]-phenyl}-acetic acid (P-0002),
(3-Methoxy-5-{3-[1-(3-methyl-butyl)-1H-pyrazol-4-yl]-benzenesulfonyl}-phenyl)-acetic acid (P-0003),
{3-[3-(1-Isobutyl-1H-pyrazol-4-yl)-benzenesulfonyl]-5-methoxy-phenyl}-acetic acid (P-0004),
{3-[3-(6-Methoxy-pyridin-3-yl)-benzenesulfonyl]-phenyl}-acetic acid (P-0005),
(3-{3-[1-(3-Methyl-butyl)-1H-pyrazol-4-yl]-benzenesulfonyl}-phenyl)-acetic acid (P-0006),
{3-[3-(1-Isobutyl-1H-pyrazol-4-yl)-benzenesulfonyl]-phenyl}-acetic acid (P-0007), and
   all salts, prodrugs, tautomers, and isomers thereof.

In one embodiment of compounds of Formula I, the compound is selected from the group consisting of:
[3-Chloro-5-(4′-trifluoromethyl-biphenyl-3-sulfonyl)-phenyl]-acetic acid (P-0089),
[3-Methyl-5-(4′-trifluoromethyl-biphenyl-3-sulfonyl)-phenyl]-acetic acid (P-0090), and
   all salts, prodrugs, tautomers, and isomers thereof.

In some embodiments, compounds of Formula I have the following sub-generic structure Formula Iq:

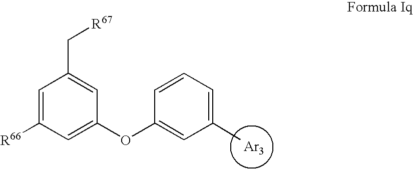

all salts, prodrugs, tautomers and isomers thereof,
wherein:
R⁶⁶ is hydrogen or methoxy;
R⁶⁷ is selected from the group consisting of —C(O)OR⁶⁸, —C(O)NR⁶⁹R⁷⁰, and a carboxylic acid isostere;
R⁶⁸ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁶⁸ is lower alkyl, any substitution on the alkyl carbon bound to the O of OR⁶⁸ is fluoro;
R⁶⁵ and R⁷⁰ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl fluoro substituted lower alkyl lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁶⁹ and/or R⁷⁰ is lower alkyl, any substitution on the alkyl carbon bound to the N of NR⁶⁹R⁷⁰ is fluoro; or
R⁶⁹ and R⁷⁰ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
Ar₃ is selected from the group consisting of:

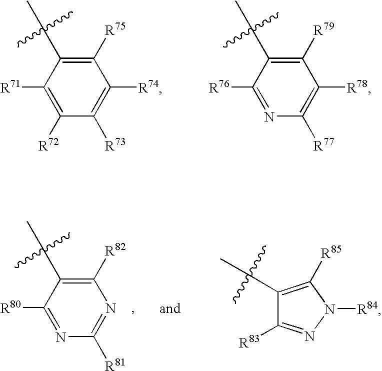

wherein

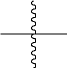

indicates the point of attachment of Ar₂ to the ring of Formula Ip;
R⁷¹, R⁷², R⁷³, R⁷⁴, R⁷⁵, R⁷⁸ and R⁷⁹ are independently selected from the group consisting of hydrogen, fluoro, chloro, C₁₋₃ alkyl, fluoro substituted C₁₋₃ alkyl, C₁₋₃ alkoxy, fluoro substituted C₁₋₃ alkoxy, and benzyloxy;
R⁷⁶, R⁷⁷, R⁸³ and R⁸⁵ are independently selected from the group consisting of hydrogen, fluoro, C₁₋₃ alkyl, fluoro substituted C₁₋₃ alkyl, C₁₋₃ alkoxy, fluoro substituted C₁₋₃ alkoxy, and benzyloxy;
R⁸⁰, R⁸¹ and R⁸² are independently selected from the group consisting of hydrogen, C₁₋₃ alkyl, fluoro substituted C₁₋₃ alkyl, C₁₋₃ alkoxy, fluoro substituted C₁₋₃ alkoxy, and benzyloxy; and
R⁸⁴ is lower alkyl or fluoro substituted lower alkyl.

In some embodiments of compounds of Formula q, Ar₃ is

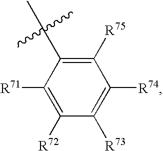

and R⁷¹, R⁷², R⁷³, R⁷⁴, and R⁷⁵ are independently selected from the group consisting of hydrogen, fluoro, chloro, methyl, trifluoromethyl, methoxy, trifluoromethoxy, ethoxy, and benzyloxy. In some embodiments, three of R⁷¹, R⁷², R⁷³, R⁷⁴, and R⁷⁵ are hydrogen and the others of R⁷¹, R⁷², R⁷³, R⁷⁴, and R⁷⁵ are independently selected from the group consisting of hydrogen, fluoro, chloro, methyl, trifluoromethyl, methoxy, trifluoromethoxy, ethoxy, and benzyloxy.

In some embodiments of compounds of Formula Iq, Ar₃ is

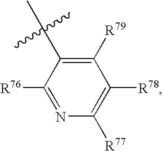

R⁷⁶, and R⁷⁷ are independently selected from the group consisting of hydrogen, fluoro, methyl, trifluoromethyl, methoxy, trifluoromethoxy, ethoxy, and benzyloxy, and R⁷⁸ and R⁷⁹ are independently selected from the group consisting of hydrogen, fluoro, chloro, methyl, trifluoromethyl, methoxy, trifluoromethoxy, ethoxy, and benzyloxy. In some embodiments R⁷⁶, R⁷⁷, R⁷⁸, and R⁷⁹ are independently selected from the group consisting of hydrogen and methoxy.

In some embodiments of compounds of Formula Iq, Ar₃ is

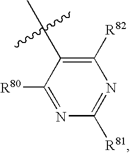

and R⁸⁰, R⁸¹, and R⁸² are independently selected from the group consisting of hydrogen, methyl, trifluoromethyl, methoxy, trifluoromethoxy, ethoxy, and benzyloxy. In some embodiments R⁸⁰, R⁸¹, and R⁸² are independently selected from the group consisting of hydrogen and methoxy.

In some embodiments of compounds of Formula Iq, Ar₃ is

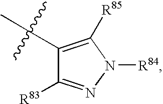

R⁸³ and R⁸⁵ are independently selected from the group consisting of hydrogen, fluoro, methyl, trifluoromethyl, methoxy, trifluoromethoxy, ethoxy, and benzyloxy, and R⁸⁴ is lower alkyl. In some embodiments, R⁸³ and R⁸⁵ are hydrogen and R⁸⁴ is lower alkyl.

In one embodiment of compounds of Formula I, the compound is selected from the group consisting of:
[3-(3′-Chloro-biphenyl-3-yloxy)-5-methoxy-phenyl]-acetic acid (P-0038),
[3-4′-Chloro-biphenyl-3-yloxy)-5-methoxy-phenyl]-acetic acid (P-0039),
[3-Methoxy-5-(4′-methoxy-biphenyl-3-yloxy)-phenyl]-acetic acid (P-0040),
[3-(4′-Fluoro-biphenyl-3-yloxy)-5-methoxy-phenyl]-acetic acid (P-0041),
[3-(3′-Chloro-4′-fluoro-biphenyl-3-yloxy)-5-methoxy-phenyl]-acetic acid (P-0042),
[3-(4′-Ethoxy-biphenyl-3-yloxy)-5-methoxy-phenyl]-acetic acid (P-0043),
[3-(3′-Fluoro-biphenyl-3-yloxy)-5-methoxy-phenyl]-acetic acid (P-0044),
[3-Methoxy-5-(3′-trifluoromethoxy-biphenyl-3-yloxy)-phenyl]-acetic acid (P-0045),
[3-Methoxy-5-(4′-trifluoromethoxy-biphenyl-3-yloxy)-phenyl]-acetic acid (P-0046),
[3-Methoxy-5-(3′-trifluoromethyl-biphenyl-3-yloxy)-phenyl]-acetic acid (P-0047).
[3-(3′-Fluoro-4′-methyl-biphenyl-3-yloxy)-5-methoxy-phenyl]-acetic acid (P-0048),
[3-(3′-Fluoro-4′-methoxy-biphenyl-3-yloxy)-5-methoxy-phenyl]-acetic acid (P-0049),
[3-(2′,4′-Difluoro-biphenyl-3-yloxy)-5-methoxy-phenyl]-acetic acid (P-0068),
[3-(2′-Fluoro-biphenyl-3-yloxy)-5-methoxy-phenyl]-acetic acid (P-0069),
[3-(4′-Benzyloxy-2′-fluoro-biphenyl-3-yloxy)-5-methoxy-phenyl]-acetic acid (P-0070),
[3-(4′-Chloro-2′-fluoro-biphenyl-3-yloxy)-5-methoxy-phenyl]-acetic acid (P-0071),
[3-(2′-Fluoro-4′-trifluoromethyl-biphenyl-3-yloxy)-5-methoxy-phenyl]-acetic acid (P-0078),
[3-(2′,5′-Dichloro-biphenyl-3-yloxy)-5-methoxy-phenyl]-acetic acid (P-0095),
[3-(2′,4′-Dichloro-biphenyl-3-yloxy)-5-methoxy-phenyl]-acetic acid (P-0096),
[3-(4′-fluoro-2′-methyl-biphenyl-3-yloxy)-5-methoxy-phenyl]-acetic acid (P-0097),
[3-(3′,4′-Difluoro-biphenyl-3-yloxy)-5-methoxy-phenyl]-acetic acid (P-0098),
[3-(2′,3′-Dichloro-biphenyl-3-yloxy)-5-methoxy-phenyl]-acetic acid (P-0099),
[3-(4′-Fluoro-3′-methyl-biphenyl-3-yloxy)-5-methoxy-phenyl]-acetic acid (P-0100),
[3-(2′,3′-Difluoro-biphenyl-3-yloxy)-5-methoxy-phenyl]-acetic acid (P-0101),
[3-(2′-fluoro-4′-methoxy-biphenyl-3-yloxy)-5-methoxy-phenyl]-acetic acid (P-0102),
[3-(4′-Fluoro-3′-trifluoromethyl-biphenyl-3-yloxy)-5-methoxy-phenyl]-acetic acid (P-0103),
[3-(4′-Chloro-2′-methyl-biphenyl-3-yloxy)-5-methoxy-phenyl]-acetic acid (P-0104),
[3-(2′-Chloro-4′-ethoxy-biphenyl-3-yloxy)-5-methoxy-phenyl]-acetic acid (P-0105),
[3-(2′-Chloro-4′-fluoro-biphenyl-3-yloxy)-5-methoxy-phenyl]-acetic acid (P-0106),
[3-(2′-Chloro-4′-trifluoromethyl-biphenyl-3-yloxy)-5-methoxy-phenyl]-acetic acid (P-0110),
[3-(5′-Chloro-2′-fluoro-biphenyl-3-yloxy)-5-methoxy-phenyl]-acetic acid (P-0111), and
   all salts, prodrutgs, tautomers, and isomers thereof.

In one embodiment of compounds of Formula I, the compound is selected from the group consisting of:
[3-(3′-Chloro-biphenyl-3-yloxy)-phenyl]-acetic acid (P-0050),
[3-(4′-Chloro-biphenyl-3-yloxy)-phenyl]-acetic acid (P-0051),
[3-(4′-Methoxy-biphenyl-3-yloxy)-phenyl]-acetic acid (P-0052),
[3-(4′-Fluoro-biphenyl-3-yloxy)-phenyl]-acetic acid (P-0053),
[3-(3′-Chloro-4′-fluoro-biphenyl-3-yloxy)-phenyl]-acetic acid (P-0054),
[3-(4′-Ethoxy-biphenyl-3-yloxy)-phenyl]-acetic acid (P-0055),
[3-(3′-Fluoro-biphenyl-3-yloxy)-phenyl]-acetic acid (P-0056),
[3-(3′-Trifluoromethoxy-biphenyl-3-yloxy)-phenyl]-acetic acid (P-0057),
[3-(4′-Trifluoromethoxy-biphenyl-3-yloxy)-phenyl]-acetic acid (P-0058),
[3-(3′-Trifluoromethyl-biphenyl-3-yloxy)-phenyl]-acetic acid (P-0059),
[3-(3′-Fluoro-4′-methyl-biphenyl-3-yloxy)-phenyl]-acetic acid (P-0060),
[3-(3′-Fluoro-4′-methoxy-biphenyl-3-yloxy)-phenyl]-acetic acid (P-0061),
[3-(2′,4′-Difluoro-biphenyl-3-yloxy)-phenyl]-acetic acid (P-0072),
[3-(2′-Fluoro-biphenyl-3-yloxy)-phenyl]-acetic acid (P-0073),
[3-(4′-Benzyloxy-2′-fluoro-biphenyl-3-yloxy)-phenyl]-acetic acid (P-0074),
[3-(2′-Fluoro-4′-methoxy-biphenyl-3-yloxy)-phenyl]-acetic acid (P-0075),
[3-(4′-Chloro-2′-fluoro-biphenyl-3-yloxy)-phenyl]-acetic acid (P-0076),
[3-(2′-Fluoro-4′-trifluoromethyl-biphenyl-3-yloxy)-phenyl]-acetic acid (P-0079),
[3-(4′-Fluoro-2′-methyl-biphenyl-3-yloxy)-phenyl]-acetic acid (P-0107),
[3-(4′-Fluoro-3′-methyl-biphenyl-3-yloxy)-phenyl]-acetic acid (P-0108),
[3-(2′-Chloro-3′-fluoro-biphenyl-3-yloxy)-phenyl]-acetic acid (P-0109),
[3-(2′-Chloro-4′-trifluoromethyl-biphenyl-3-yloxy)-phenyl]-acetic acid (P-0112),
[3-(5′-Chloro-2′-fluoro-biphenyl-3-yloxy)-phenyl]-acetic acid (P-0113),
[3-(2′,5′-Dichloro-biphenyl-3-yloxy)-phenyl]-acetic acid (P-0114),
[3-(2′,4′-Dichloro-biphenyl-3-yloxy)-phenyl]-acetic acid (P-0115),
[3-(2′,3′-Dichloro-biphenyl-3-yloxy)-phenyl]-acetic acid (P-0116),
[3-(3′,4′-Dichloro-biphenyl-3-yloxy)-phenyl]-acetic acid (P-0117),
[3-(4′-Fluoro-3′-trifluoromethyl-biphenyl-3-yloxy)-phenyl]-acetic acid (P-0118),
[3-(4′-Chloro-2′-methyl-biphenyl-3-yloxy)-phenyl]-acetic acid (P-0119),
[3-(2′-Chloro-4′-fluoro-biphenyl-3-yloxy)-phenyl]-acetic acid (P-0120),
[3-(3′,4′-Difluoro-biphenyl-3-yloxy)-phenyl]-acetic acid (P-0121),
[3-(2′,3′-Difluoro-biphenyl-3-yloxy)-phenyl]-acetic acid (P-0122),
[3-(4′-Fluoro-3′-methoxy-biphenyl-3-yloxy)-phenyl]-acetic acid (P-0123),
[3-(2′-Chloro-4′-ethoxy-biphenyl-3-yloxy)-phenyl]-acetic acid (P-0124),
[3-(4′-Ethoxy-2′-methyl-biphenyl-3-yloxy)-phenyl]-acetic acid (P-0125), and
   all salts, prodrugs, tautomers, and isomers thereof.

In one embodiment of compounds of Formula I, the compound is selected from the group consisting of:
{3-Methoxy-5-[3-(6-methoxy-pyridin-3-yl)-phenoxy]-phenyl}-acetic acid (P-0008),
{3-Methoxy-5-[3-(2-methoxy-pyrimidin-5-yl)-phenoxy]-phenyl}-acetic acid (P-0009),
{3-[3-(2,4-Dimethoxy-pyrimidin-5-yl)-phenoxy]-5-methoxy-phenyl}-acetic acid (P-0010),
(3-Methoxy-5-{3-[1-(3-methyl-butyl)-1H-pyrazol-4-yl]-phenoxy}-phenyl)-acetic acid (P-0011),
{3-[3-(1-Isobutyl-1H-pyrazol-4-yl)-phenoxy]-5-methoxy-phenyl}-acetic acid (P-0012),
{3-[3-(6-Methoxy-pyridin-3-yl)-phenoxy]-phenyl}-acetic acid (P-0013),
{3-[3-(2-Methoxy-pyrimidin-5-yl)-phenoxy]-phenyl}-acetic acid (P-0014),
{3-[3-(1-Isobutyl-1H-pyrazol-4-yl)-phenoxy]-phenyl}-acetic acid (P-0077), and
   all salts, prodrugs, tautomers, and isomers thereof.

In some embodiments of the above compounds, compounds are excluded where N (except where N is a heteroaryl ring atom), O, or S is bound to a carbon that is also bound to N (except where N is a heteroaryl ring atom), O, or S, except where the carbon forms a double bond with one of the heteroatoms, such as in an amide, carboxylic acid, and the like; or where N (except where N is a heteroaryl ring atom), O, C(S), C(O), or S(O)ₙ (n is 0-2) is bound to an alkene carbon of an alkenyl group or bound to an alkyne carbon of an alkynyl group; accordingly, in some embodiments compounds that include linkages such as the following are excluded from the present invention: —NR—CH₂—NR—, —O—CH₂—NR—, —S—CH₂—NR—, —NR—CH₂—O—, —O—CH₂—O—, —S—CH₂—O—, —NR—CH₂—S—, —O—CH₂—S—, —S—CH₂—S—, —NR—CH═CH—, —CH═CH—NR—, —NR—C≡C—, —C≡C—NR—, —O—CH═CH—, —CH═CH—O—, —O—C≡C—, —C≡C—O—, —S(O)₀₋₂—CH═CH—, —CH═CH—S(O)₀₋₂—, —S(O)₀₋₂—C≡C—, —C≡C—S(O)₀₋₂—, —C(O)—CH═CH—, —CH═CH—C(O)—, —C≡C—C(O)—, —C(O)—C≡C—, —C(S)—CH═CH—, —CH═CH—C(S)—, —C≡C—C(S)—, or —C(S)—C≡C—.

Reference to compounds of Formula I herein includes specific reference to sub-groups and species of compounds of Formula I described herein (e.g., including Formulae Ia-Iq, and all embodiments as described above) unless indicated to the contrary. In specifying a compound or compounds of Formula I, unless clearly indicated to the contrary, specification of such compound(s) includes pharmaceutically acceptable salts of the compound(s), pharmaceutically acceptable formulations of the compound(s), prodrug(s), and all stereoisomers thereof.

Another aspect of this invention provides compositions that include a therapeutically effective amount of a compound of Formula I and at least one pharmaceutically acceptable carrier, excipient, and/or diluent. The composition can include a plurality of different pharmacologically active compounds, including one or more compounds of Formula I.

In another aspect, compounds of Formula I can be used in the preparation of a medicament for the treatment of a PPAR-mediated disease or condition or a disease or condition in which modulation of a PPAR provides a therapeutic benefit. In a further aspect, the disease or condition is selected from the group consisting of weight disorders (e.g., including, but not limited to, obesity, overweight condition, bulimia, and anorexia nervosa), lipid disorders (e.g., including, but not limited to, hyperlipidemia, dyslipidemia (including associated diabetic dyslipidemia and mixed dyslipidemia), hypoalphalipoproteinemia, hypertriglyceridemia, hypercholesterolemia, and low HDL (high density lipoprotein)), metabolic disorders (e.g., including, but not limited to, Metabolic Syndrome, Type II diabetes mellitus, Type I diabetes, hyperinsulinemia, impaired glucose tolerance, insulin resistance, diabetic complication (e.g., including, but not limited to, neuropathy, nephropathy, retinopathy, diabetic foot ulcer, bladder dysfunction, bowel dysfunction, diaphragmatic dysfunction and cataracts)), cardiovascular disease (e.g., including, but not limited to, hypertension, coronary heart disease, heart failure, congestive hear failure, atherosclerosis, arteriosclerosis, stroke, cerebrovascular disease, myocardial infarction, and peripheral vascular disease), inflammatory diseases (e.g., including, but not limited to, autoimmune diseases (e.g., including, but not limited to, vitiligo, uveitis, optic neuritis, pemphigus foliaceus, pemphigoid, inclusion body myositis, polymyositis, dermatomyositis, scleroderma, Grave's disease, Hashimoto's disease, chronic graft versus host disease, ankylosing spondylitis, rheumatoid arthritis, inflammatory bowel disease (e.g. ulcerative colitis, Crohn's disease), systemic lupus erythematosis, Sjogren's Syndrome, and multiple sclerosis), diseases involving airway inflammation (e.g., including, but not limited to, asthma and chronic obstructive pulmonary disease), inflammation in other organs (e.g., including, but not limited to, polycystic kidney disease (PKD), polycystic ovary syndrome, pancreatitis, nephritis, and hepatitis), otitis, stomatitis, sinusitis, arteritis, temporal arteritis, giant cell arteritis, and polymyalgia rheumatica), skin disorders (e.g., including, but not limited to, epithelial hyperproliferative diseases (e.g., including, but not limited to, eczema and psoriasis), dermatitis (e.g., including, but not limited to, atopic dermatitis, contact dermatitis, allergic dermatitis and chronic dermatitis), and impaired wound healing)), neurodegenerative disorders (e.g., including, but not limited to, Alzheimer's disease, Parkinson's disease, amyotrophic lateral sclerosis, spinal cord injury, and demyelinating disease (e.g., including, but not limited to, acute disseminated encephalomyelitis and Guillain-Barre syndrome)), coagulation disorders (e.g., including, but not limited to, thrombosis), gastrointestinal disorders (e.g., including, but not limited to, gastroesophageal reflux, appendicitis, diverticulitis, gastrointestinal ulcers, ileus, motility disorders and infarction of the large or small intestine), genitourinary disorders (e.g., including, but not limited to, renal insufficiency, erectile dysfunction, urinary incontinence, and neurogenic bladder), ophthalmic disorders (e.g., including, but not limited to, ophthalmic inflammation, conjunctivitis, keratoconjunctivitis, corneal inflammation, dry eye syndrome, macular degeneration, and pathologic neovascularization), infections (e.g., including, but not limited to, lyme disease, HCV, HIV, and Helicobacter pylori) and inflammation associated with infections (e.g., including, but not limited to, encephalitis, meningitis), neuropathic or inflammatory pain, pain syndromes (e.g., including, but not limited to, chronic pain syndrome, fibromyalgia), infertility, and cancer (e.g., including, but not limited to, breast cancer and thyroid cancer).

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of weight disorders, lipid disorders, metabolic disorders and cardiovascular disease. In some embodiments, the disease or condition is selected from the group consisting of obesity, dyslipidemia, Metabolic Syndrome, Type II diabetes mellitus and atherosclerosis.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of inflammatory disease, neurodegenerative disorder, coagulation disorder, gastrointestinal disorder, genitourinary disorder, ophthalmic disorder, infection, inflammation associated with infection, neuropathic pain, inflammatory pain, pain syndromes, infertility and cancer. In some embodiments, the disease or condition is selected from the group consisting of inflammatory disease, neurodegenerative disorder, and cancer. In some embodiments, the disease or condition is selected from the group consisting of inflammatory bowel disease, multiple sclerosis, Alzheimer's disease, breast cancer and thyroid cancer.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of weight disorders, lipid disorders and cardiovascular disease.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of metabolic disorders, inflammatory diseases and neurodegenerative diseases.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of ophthalmic disorders, infections and inflammation associated with infections.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of neuropathic pain, inflammatory pain and pain syndromes.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of infertility and cancer.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of Metabolic Syndrome, Type II diabetes mellitus, Type I diabetes, hyperinsulinemia, impaired glucose tolerance, insulin resistance and a diabetic complication selected from the group consisting of neuropathy, nephropathy, retinopathy, diabetic foot ulcer, bladder dysfunction, bowel dysfunction, diaphragmatic dysfunction and cataracts, preferably the disease or condition is Metabolic Syndrome or Type II diabetes mellitus.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of obesity, overweight condition, bulimia, anorexia nervosa, hyperlipidemia, dyslipidemia, hypoalphalipoproteinemia, hypentriglyceridemia, hypercholesterolemia, and low HDL, preferably the disease or condition is obesity or dyslipidemia.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of Alzheimer's disease, Parkinson's disease, amyotrophic lateral sclerosis, spinal cord injury, and demyelinating disease, preferably the disease or condition is Alzheimer's disease.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of vitiligo, uveitis, optic neuritis, pemphigus foliaceus, pemphigoid, inclusion body myositis, polymyositis, dermatomyositis, scleroderma, Grave's disease, Hashimoto's disease, chronic graft versus host disease, ankylosing spondylitis, rheumatoid arthritis, inflammatory bowel disease systemic lupus erythematosis, Sjogren's Syndrome, and multiple sclerosis, asthma, chronic obstructive pulmonary disease, polycystic kidney disease, polycystic ovary syndrome, pancreatitis, nephritis, hepatitis, otitis, stomatitis, sinusitis, arteritis, temporal arteritis, giant cell arteritis, polymyalgia rheumatica, eczema, psoriasis, atopic dermatitis, contact dermatitis, allergic dermatitis, chronic dermatitis, and impaired wound healing, preferably the disease or condition is inflammatory bowel disease or multiple sclerosis.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of infertility and cancer, preferably the disease or condition is breast or thyroid cancer.

In some embodiments, compounds of Formula I can be used in the preparation of a medicament for the treatment of a PPAR-mediated disease or condition or a disease or condition in which modulation of a PPAR provides a therapeutic benefit, wherein the disease or condition is selected from the group consisting of hypertension, coronary heart disease, heart failure, congestive heart failure, atherosclerosis, arteriosclerosis, stroke, cerebrovascular disease, myocardial infarction, and peripheral vascular disease, preferably the disease or condition is atherosclerosis.

In another aspect, the invention provides a kit that includes a compound of Formula I or a composition thereof as described herein. In some embodiments, the compound or composition is packaged, e.g., in a vial, bottle, flask, which may be further packaged, e.g., within a box, envelope, or bag. In some embodiments, the compound or composition is approved by the U.S. Food and Drug Administration or similar regulatory agency for administration to a mammal, e.g., a human. In some embodiments, the compound or composition is approved for administration to a mammal, e.g., a human for a PPAR-mediated disease or condition or a disease or condition in which modulation of a PPAR provides a therapeutic benefit. In some embodiments, the kit includes written instructions or other indication that the compound or composition is suitable or approved for administration to a mammal, e.g., a human, for a PPAR-mediated disease or condition or a disease or condition in which modulation of a PPAR provides a therapeutic benefit. In some embodiments, the compound or composition is packaged in unit dose or single dose form, e.g., single dose pills, capsules, or the like.

In another aspect, the invention provides a method of treating or prophylaxis of a disease or condition in an animal subject, e.g., a PPAR-mediated disease or condition or a disease or condition in which modulation of a PPAR provides a therapeutic benefit, by administering to the subject a therapeutically effective amount of a compound of Formula I, a prodrug of such compound, a pharmaceutically acceptable salt of such compound or prodrug, or a pharmaceutically acceptable formulation of such compound or prodrug. The compound can be administered alone or can be administered as part of a pharmaceutical composition. In one aspect, the method involves administering to the subject an effective amount of a compound of Formula I in combination with one or more other therapies for the disease or condition.

In another aspect, the invention provides a method of treating or prophylaxis of a PPAR-mediated disease or condition or a disease or condition in which modulation of a PPAR provides a therapeutic benefit, wherein the method involves administering to the subject a therapeutically effective amount of a composition including a compound of Formula I.

In aspects and embodiments involving treatment or prophylaxis of a PPAR-mediated disease or condition, or a disease or condition in which modulation of a PPAR provides a therapeutic benefit, the disease or condition is selected from the group consisting of weight disorders (e.g., including, but not limited to, obesity, overweight condition, bulimia, and anorexia nervosa), lipid disorders (e.g., including, but not limited to, hyperlipidemia, dyslipidemia (including associated diabetic dyslipidemia and mixed dyslipidemia), hypoalphalipoproteinemia, hypertriglyceridemia, hypercholesterolemia, and low HDL (high density lipoprotein)), metabolic disorders (e.g., including, but not limited to, Metabolic Syndrome, Type II diabetes mellitus, Type I diabetes, hyperinsulinemia, impaired glucose tolerance, insulin resistance, diabetic complication (e.g., including, but not limited to, neuropathy, nephropathy, retinopathy, diabetic foot ulcer, bladder dysfunction, bowel dysfunction, diaphragmatic dysfunction and cataracts)), cardiovascular disease (e.g., including, but not limited to, hypertension, coronary heart disease, heart failure, congestive heart failure, atherosclerosis, arteriosclerosis, stroke, cerebrovascular disease, myocardial infarction, and peripheral vascular disease), inflammatory diseases (e.g., including, but not limited to, autoimmune diseases (e.g., including, but not limited to, vitiligo, uveitis, optic neuritis, pemphigus foliaceus, pemphigoid, inclusion body myositis, polymyositis, dermatomyositis, scleroderma, Grave's disease, Hashimoto's disease, chronic graft versus host disease, ankylosing spondylitis, rheumatoid arthritis, inflammatory bowel disease (e.g., ulcerative colitis, Crohn's disease), systemic lupus erythematosis, Sjogren's Syndrome, and multiple sclerosis), diseases involving airway inflammation (e.g., including, but not limited to, asthma and chronic obstructive pulmonary disease), inflammation in other organs (e.g., including, but not limited to, polycystic kidney disease (PKD), polycystic ovary syndrome, pancreatitis, nephritis, and hepatitis), otitis, stomatitis, sinusitis, arteritis, temporal arteritis, giant cell arteritis, and polymyalgia rheumatica), skin disorders (e.g., including, but not limited to, epithelial hyperproliferative diseases (e.g., including, but not limited to, eczema and psoriasis), dermatitis (e.g., including, but not limited to, atopic dermatitis, contact dermatitis, allergic dermatitis and chronic dermatitis), and impaired wound healing)), neurodegenerative disorders (e.g., including, but not limited to, Alzheimer's disease, Parkinson's disease, amyotrophic lateral sclerosis, spinal cord injury, and demyelinating disease (e.g., including, but not limited to, acute disseminated encephalomyelitis and Guillain-Barre syndrome)), coagulation disorders (e.g., including, but not limited to, thrombosis), gastrointestinal disorders (e.g., including, but not limited to, gastroesophageal reflux, appendicitis, diverticulitis, gastrointestinal ulcers, ileus, motility disorders and infarction of the large or small intestine), genitourinary disorders (e.g., including, but not limited to, renal insufficiency, erectile dysfunction, urinary incontinence, and neurogenic bladder), ophthalmic disorders (e.g., including, but not limited to, ophthalmic inflammation, conjunctivitis, keratoconjunctivitis, corneal inflammation, dry eye syndrome, macular degeneration, and pathologic neovascularization), infections (e.g., including, but not limited to, lyme disease, HCV, HIV, and Helicobacter pylori) and inflammation associated with infections (e.g., including, but not limited to, encephalitis, meningitis), neuropathic or inflammatory pain, pain syndromes (e.g., including, but not limited to, chronic pain syndrome, fibromyalgia), infertility, and cancer (e.g., including, but not limited to, breast cancer and thyroid cancer).

In some embodiments involving treatment or prophylaxis of a PPAR-mediated disease or condition, or a disease or condition in which modulation of a PPAR provides a therapeutic benefit, the disease or condition is selected from the group consisting of weight disorders, lipid disorders, metabolic disorders and cardiovascular disease. In some embodiments, the disease or condition is selected from the group consisting of obesity, dyslipidemia, Metabolic Syndrome, Type II diabetes mellitus and atherosclerosis.

In some embodiments involving treatment or prophylaxis of a PPAR-mediated disease or condition, or a disease or condition in which modulation of a PPAR provides a therapeutic benefit, the disease or condition is selected from the group consisting of inflammatory disease, neurodegenerative disorder, coagulation disorder, gastrointestinal disorder, genitourinary disorder, ophthalmic disorder, infection, inflammation associated with infection, neuropathic pain, inflammatory pain, pain syndromes, infertility and cancer. In some embodiments, the disease or condition is selected from the group consisting of inflammatory disease, neurodegenerative disorder, and cancer. In some embodiments, the disease or condition is selected from the group consisting of inflammatory bowel disease, multiple sclerosis, Alzheimer's disease, breast cancer and thyroid cancer.

In some embodiments involving treatment or prophylaxis of a PPAR-mediated disease or condition, or a disease or condition in which modulation of a PPAR provides a therapeutic benefit, the disease or condition is selected from the group consisting of weight disorders, lipid disorders and cardiovascular disease.

In some embodiments involving treatment or prophylaxis of a PPAR-mediated disease or condition, or a disease or condition in which modulation of a PPAR provides a therapeutic benefit, the disease or condition is selected from the group consisting of metabolic disorders, inflammatory diseases and neurodegenerative diseases.

In some embodiments involving treatment or prophylaxis of a PPAR-mediated disease or condition, or a disease or condition in which modulation of a PPAR provides a therapeutic benefit, the disease or condition is selected from the group consisting of ophthalmic disorders, infections and inflammation associated with infections.

In some embodiments involving treatment or prophylaxis of a PPAR-mediated disease or condition, or a disease or condition in which modulation of a PPAR provides a therapeutic benefit, the disease or condition is selected from the group consisting of neuropathic pain, inflammatory pain and pain syndromes.

In some embodiments involving treatment or prophylaxis of a PPAR-mediated disease or condition, or a disease or condition in which modulation of a PPAR provides a therapeutic benefit, the disease or condition is selected from the group consisting of infertility and cancer.

In some embodiments involving treatment or prophylaxis of a PPAR-mediated disease or condition, or a disease or condition in which modulation of a PPAR provides a therapeutic benefit, the disease or condition is selected from the group consisting of Metabolic Syndrome, Type II diabetes mellitus, Type I diabetes, hyperinsulinemia, impaired glucose tolerance, insulin resistance and a diabetic complication selected from the group consisting of neuropathy, nephropathy, retinopathy, diabetic foot ulcer, bladder dysfunction, bowel dysfunction, diaphragmatic dysfunction and cataracts, preferably the disease or condition is Metabolic Syndrome or Type II diabetes mellitus.

In some embodiments involving treatment or prophylaxis of a PPAR-mediated disease or condition, or a disease or condition in which modulation of a PPAR provides a therapeutic benefit, the disease or condition is selected from the group consisting of obesity, overweight condition, bulimia, anorexia nervosa, hyperlipidemia, dyslipidemia, hypoalphalipoproteinemia, hypertriglyceridemia, hypercholesterolemia, and low HDL, preferably the disease or condition is obesity or dyslipidemia.

In some embodiments involving treatment or prophylaxis of a PPAR-mediated disease or condition, or a disease or condition in which modulation of a PPAR provides a therapeutic benefit, the disease or condition is selected from the group consisting of Alzheimer's disease, Parkinson's disease, amyotrophic lateral sclerosis, spinal cord injury, and demyelinating disease, preferably the disease or condition is Alzheimer's disease.

In some embodiments involving treatment or prophylaxis of a PPAR-mediated disease or condition, or a disease or condition in which modulation of a PPAR provides a therapeutic benefit, the disease or condition is selected from the group consisting of vitiligo, uveitis, optic neuritis, pemphigus foliaceus, pemphigoid, inclusion body myositis, polymyositis, dermatomyositis, scleroderma, Grave's disease, Hashimoto's disease, chronic graft versus host disease, ankylosing spondylitis, rheumatoid arthritis, inflammatory bowel disease systemic lupus erythematosis, Sjogren's Syndrome, and multiple sclerosis, asthma, chronic obstructive pulmonary disease, polycystic kidney disease, polycystic ovary syndrome, pancreatitis, nephritis, hepatitis, otitis, stomatitis, sinusitis, arteritis, temporal arteritis, giant cell arteritis, polymyalgia rheumatica, eczema, psoriasis, atopic dermatitis, contact dermatitis, allergic dermatitis, chronic dermatitis, and impaired wound healing, preferably the disease or condition is inflammatory bowel disease or multiple sclerosis.

In some embodiments involving treatment or prophylaxis of a PPAR-mediated disease or condition, or a disease or condition in which modulation of a PPAR provides a therapeutic benefit, the disease or condition is selected from the group consisting of infertility and cancer, preferably the disease or condition is breast or thyroid cancer.

In some embodiments involving treatment or prophylaxis of a PPAR-mediated disease or condition, or a disease or condition in which modulation of a PPAR provides a therapeutic benefit, the disease or condition is selected from the group consisting of hypertension, coronary heart disease, heart failure, congestive heart failure, atherosclerosis, arteriosclerosis, stroke, cerebrovascular disease, myocardial infarction, and peripheral vascular disease, preferably the disease or condition is atherosclerosis.

In some embodiments of aspects involving compounds of Formula I, the compound is specific for any one or any two of PPARα, PPAR-γ and PPARδ, e.g. specific for PPARα; specific for PPARδ; specific for PPARγ; specific for PPARα and PPARδ; specific for PPARα and PPARγ; or specific for PPARδ and PPARγ. In some embodiments, compounds are preferably specific for PPARδ. In some embodiments, compounds are preferably specific for PPARγ and PPARδ. In some embodiments, compounds are preferably specific for PPARα and PPARδ. Such specificity means that the compound has at least 5-fold greater activity (preferably at least 10-, 20-, 50-, or 100-fold or more greater activity) on the specific PPAR(s) than on the other PPAR(s), where the activity is determined using a biochemical assay suitable for determining PPAR activity, e.g., any assay known to one skilled in the art or as described herein. In some embodiments, compounds have significant activity on all three of PPARα, PPARδ, and PPARγ.

In some embodiments, a compound of Formula I will have an EC₅₀ of less than 100 nM, less than 50 nM, less than 20 nM, less than 10 nM, less than 5 nM, or less than 1 nM with respect to at least one of PPARα, PPARγ and PPARδ as determined in a generally accepted PPAR activity assay. In some embodiments, a compound of Formula I will have an EC₅₀ of less than 100 nM, less than 50 nM, less than 20 nM, less than 10 nM, less than 5 nM, or less than 1 nM with respect to at least any two of PPARα, PPARγ and PPARδ. In some embodiments, a compound of Formula I will have an EC₅₀ of less than 100 nM, less than 50 nM, less than 20 nM, less than 10 nM, less than 5 nM, or less than 1 nM with respect to all three of PPARα, PPARγ and PPARδ. In some embodiments, a compound of the invention may be a specific agonist of any one of PPARα, PPARγ and PPARδ, or any two of PPARα, PPARγ and PPARδ. In some embodiments, a compound of the invention will preferably have an EC₅₀ of less than 100 nM, less than 50 nM, less than 20 nM, less than 10 nM, less than 5 nM, or less than 1 nM with respect to at least PPARδ as determined in a generally accepted PPAR activity assay. In some embodiments, a compound of the invention will preferably have an EC₅₀ of less than 100 nM, less than 50 nM, less than 20 nM, less than 10 nM, less than 5 nM, or less than 1 nM with respect to PPARδ and PPARγ as determined in a generally accepted PPAR activity assay. In some embodiments, a compound of the invention will preferably have an EC₅₀ of less than 100 nM, less than 50 nM, less than 20 nM, less than 10 nM, less than 5 nM, or less than 1 nM with respect to PPARδ and PPARα as determined in a generally accepted PPAR activity assay. A specific agonist of one of PPARα, PPARγ and PPARδ is such that the EC₅ for one of PPARα, PPARγ and PPARδ will be at least about 5-fold, also 10-fold, also 20-fold, also 50-fold, or at least about 100-fold less than the EC₅₀ for the other two of PPARα, PPARγ and PPARδ. A specific agonist of two of PPARα, PPARγ and PPARδ is such that the EC₅₀ for each of two of PPARα, PPARγ and PPARδ will be at least about 5-fold, also 10-fold, also 20-fold, also 50-fold, or at least about 100-fold less than the EC₅₀ for the other of PPARα, PPARγ and PPARδ.

In some embodiments of the invention, the compounds of Formula I active on PPARs also have desirable pharmacologic properties. In some embodiments the desired pharmacologic property is PPAR pan-activity, PPAR selectivity for any individual PPAR (PPARα, PPARδ, or PPARγ), selectivity on any two PPARs (PPARα and PPARδ, PPARα and PPARγ, or PPARδ and PPARγ), or any one or more of serum half-life longer than 2 hr, also longer than 4 hr, also longer than 8 hr, aqueous solubility, and oral bioavailability more than 10%, also more than 20%.

Additional embodiments will be apparent from the Detailed Description of the Invention and from the claims.

### DETAILED DESCRIPTION OF THE INVENTION

As indicated in the Summary of the Invention above, the present invention concerns the peroxisome proliferator-activated receptors (PPARs), which have been identified in humans and other mammals. A group of compounds have been identified, corresponding to Formula I, that are active on one or more of the PPARs, in particular compounds that are active on one or more human PPARs. Such compounds can be used as agonists on PPARs, including agonists of at least one of PPARα, PPARδ, and PPARγ, as well as dual PPAR agonists and pan-agonist, such as agonists of both PPARα and PPARγ, both PPARα and PPARδ, both PPARγ and PPARδ, or agonists of PPARα, PPARγ and PPARδ.

As used herein the following definitions apply unless otherwise indicated:

“Halogen”—alone or in combination refers to all halogens, that is, chloro (Cl), fluoro (F), bromo (Br), or iodo (I).

“Hydroxyl” or “hydroxy” refers to the group —OH.

“Thiol” refers to the group —SH.

“Lower alkyl” alone or in combination means an alkane-derived radical containing from 1 to 6 carbon atoms (unless specifically defined) that includes a straight chain alkyl or branched alkyl. The straight chain or branched alkyl group is attached at any available point to produce a stable compound. In many embodiments, a lower alkyl is a straight or branched alkyl group containing from 1-6, 1-4, or 1-2, carbon atoms, such as methyl, ethyl, propyl, isopropyl, butyl, t-butyl, and the like. “Substituted lower alkyl” denotes lower alkyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(N)NR^{b}R^{c}, —NR^{a}C(O)R^{a}, —NR^{a}C(S)R^{a}, —NR^{a}S(O)₂R^{a}NR^{a}C(O)NR^{a}R^{a}, —NR^{a}C(S)NR^{a}R^{a}, —NR^{a}S(O)₂NR^{a}R^{a}, —NR^{a}R^{a}, —R^{e} and —R^{f}. Furthermore, possible substitutions include subsets of these substitutions, such as are indicated herein, for example, in the description of compounds of Formula I, attached at any available atom to produce a stable compound. For example “fluoro substituted lower alkyl” denotes a lower alkyl group substituted with one or more fluoro atoms, such as perfluoroalkyl, where preferably the lower alkyl is substituted with 1, 2, 3, 4 or 5 fluoro atoms, also 1, 2, or 3 fluoro atoms. It is understood that substitutions are attached at any available atom to produce a stable compound, when optionally substituted lower alkyl is an R group of a moiety such as —OR (e.g. lower alkoxy), —SR (e.g. lower alkylthio), —NHR (e.g. mono-alkylamino), —C(O)NHR, and the like, substitution of the lower alkyl R group is preferably such that substitution of the lower alkyl carbon bound to any O, S, or N of the moiety (except where N is a heteroaryl ring atom) excludes substituents that would result in any O, S, or N of the substituent (except where N is a heteroaryl ring atom) being bound to the lower alkyl carbon bound to any O, S, or N of the moiety.

“Lower alkenyl” alone or in combination means a straight or branched hydrocarbon containing 2-6 carbon atoms (unless specifically defined) and at least one, preferably 1-3, more preferably 1-2, most preferably one, carbon to carbon double bond. Carbon to carbon double bonds may be contained within either a straight chain or branched portion. Examples of lower alkenyl groups include ethenyl, propenyl, isopropenyl, butenyl, and the like. “Substituted lower alkenyl” denotes lower alkenyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, and —R^{f}. Further, possible substitutions include subsets of these substitutions, such as are indicated herein, for example, in the description of compounds of Formula I, attached at any available atom to produce a stable compound. It is understood that substitutions are attached at any available atom to produce a stable compound, substitution of lower alkenyl groups are preferably such that F, C(O), C(S), C(NH), S(O), S(O)₂, O, S, or N (except where N is a heteroaryl ring atom), are not bound to an alkene carbon thereof. Further, where lower alkenyl is a substituent of another moiety or an R group of a moiety such as —OR, —NHR, —C(O)R, and the like, substitution of the moiety is preferably such that any C(O), C(S), S(O), S(O)₂, O, S, or N thereof (except where N is a heteroaryl ring atom) are not bound to an alkene carbon of the lower alkenyl substituent or R group. Further, where lower alkenyl is a substituent of another moiety or an R group of a moiety such as —OR, —NHR, —C(O)NHR, and the like, substitution of the lower alkenyl R group is preferably such that substitution of the lower alkenyl carbon bound to any O, S, or N of the moiety (except where N is a heteroaryl ring atom) excludes substituents that would result in any O, S, or N of the substituent (except where N is a heteroaryl ring atom) being bound to the lower alkenyl carbon bound to any O, S, or N of the moiety. An “alkenyl carbon” refers to any carbon within a lower alkenyl group, whether saturated or part of the carbon to carbon double bond. An “alkene carbon” refers to a carbon within a lower alkenyl group that is part of a carbon to carbon double bond. “C₃₋₆ alkenyl” denotes lower alkenyl containing 3-6 carbon atoms. A “substituted C₃₋₆ alkenyl” denotes optionally substituted lower alkenyl containing 3-6 carbon atoms.

“Lower alkynyl” alone or in combination means a straight or branched hydrocarbon containing 2-6 carbon atoms (unless specifically defined) containing at least one, preferably one, carbon to carbon triple bond. Examples of lower alkynyl groups include ethynyl, propynyl, butynyl, and the like. “Substituted lower alkynyl” denotes lower alkynyl that is independently substituted, unless indicated otherwise, with one or more, preferably 1, 2, 3, 4 or 5, also 1, 2, or 3 substituents, attached at any available atom to produce a stable compound, wherein the substituents are selected from the group consisting of —F, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, and —R^{f}. Further, possible substitutions include subsets of these substitutions, such as are indicated herein, for example, in the description of compounds of Formula I, attached at any available atom to produce a stable compound. It is understood that substitutions are attached at any available atom to produce a stable compound, substitution of lower alkynyl groups are preferably such that F, C(O), C(S), C(N), S(O), S(O)₂, O, S, or N (except where N is a heteroaryl ring atom) are not bound to an alkyne carbon thereof. Further, where lower alkynyl is a substituent of another moiety or an R group of a moiety such as —OR, —NHR, —C(O)R, and the like, substitution of the moiety is preferably such that any C(O), C(S), S(O), S(O)₂, O, S, or N thereof (except where N is a heteroaryl ring atom) are not bound to an alkyne carbon of the lower alkynyl substituent or R group. Further, where lower alkynyl is a substituent of another moiety or an R group of a moiety such as —OR, —NHR, —C(O)NHR, and the like, substitution of the lower alkynyl R group is preferably such that substitution of the lower alkynyl carbon bound to any O, S, or N of the moiety (except where N is a heteroaryl ring atom) excludes substituents that would result in any O, S, or N of the substituent (except where N is a heteroaryl ring atom) being bound to the lower alkynyl carbon bound to any O, S, or N of the moiety. An “alkynyl carbon” refers to any carbon within a lower alkynyl group, whether saturated or part of the carbon to carbon triple bond. An “alkyne carbon” refers to a carbon within a lower alkynyl group that is part of a carbon to carbon triple bond. “C₃₋₆ alkynyl” denotes lower alkynyl containing 3-6 carbon atoms. A “substituted C₃₋₆ alkynyl” denotes optionally substituted lower alkynyl containing 3-6 carbon atoms.

“Carboxylic acid isostere” refers to a moiety that mimics a carboxylic acid by virtue of similar physical properties, including but not limited to molecular size, charge distribution or molecular shape. Exemplary carboxylic acid isosteres are selected from the group consisting of thiazolidine dione (i.e.

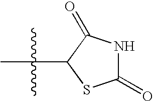

), hydroxamic acid (i.e. —C(O)NHOH), acyl-cyanamide (i.e. —C(O)NHCN), tetrazole (i.e.

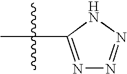

), 3- or 5-hydroxy isoxazole (i.e.

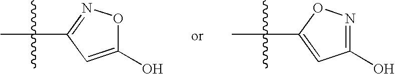

), 3- or 5-hydroxy isothiazole (i.e.

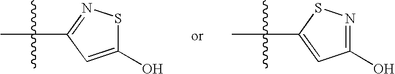

), sulphonate (i.e. —S(O)₂OH), and sulfonamide (i.e. —S(O)₂NH₂). 3- or 5-hydroxy isoxazole or 3- or 5-hydroxy isothiazole may be optionally substituted at either or both of the ring CH or the OH group with lower alkyl or lower alkyl substituted with 1, 2 or 3 substituents selected from the group consisting of fluoro, aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio. The nitrogen of the sulfonamide may be optionally substituted with a substituent selected from the group consisting of lower alkyl, fluoro substituted lower alkyl, acetyl (i.e. —C(O)CH₃), aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio.

“Aryl” alone or in combination refers to a monocyclic or bicyclic ring system containing aromatic hydrocarbons such as phenyl or naphthyl, which may be optionally fused with a cycloalkyl or heterocycloalkyl of preferably 5-7, more preferably 5-6, ring members. “Arylene” refers to a divalent aryl.

“Heteroaryl” alone or in combination refers to a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. Examples of heteroaryl groups include, but are not limited to, pyridinyl, pyridazinyl, pyrazinyl, quinoxalinyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. “Nitrogen containing heteroaryl” refers to heteroaryl wherein any heteroatoms are N. “Heteroarylene” refers to a divalent heteroaryl.

“Cycloalkyl” refers to saturated or unsaturated, non-aromatic monocyclic, bicyclic or tricyclic carbon ring systems of 3-10, also 3-8, more preferably 3-6, ring members per ring, such as cyclopropyl, cyclopentyl, cyclohexyl, adamantyl, and the like.

“Heterocycloalkyl” refers to a saturated or unsaturated non-aromatic cycloalkyl group having from 5 to 10 atoms in which from 1 to 3 carbon atoms in the ring are replaced by heteroatoms of O, S or N, and are optionally fused with benzo or heteroaryl of 5-6 ring members. Heterocycloalkyl is also intended to include oxidized S or N, such as sulfinyl, sulfonyl and N-oxide of a tertiary ring nitrogen. Heterocycloalkyl is also intended to include compounds in which one of the ring carbons is oxo substituted, i.e. the ring carbon is a carbonyl group, such as lactones and lactams. The point of attachment of the heterocycloalkyl ring is at a carbon or nitrogen atom such that a stable ring is retained. Examples of heterocycloalkyl groups include, but are not limited to, morpholino, tetrahydrofuranyl, dihydropyridinyl, piperidinyl, pyrrolidinyl, pyrrolidonyl, piperazinyl, dihydrobenzofuryl, and dihydroindolyl.

“Optionally substituted aryl”, “optionally substituted heteroaryl”, “optionally substituted cycloalkyl”, and “optionally substituted heterocycloalkyl”, refers to aryl, heteroaryl, cycloalkyl and heterocycloalkyl groups, respectively, which are optionally independently substituted, unless indicated otherwise, with one or more, preferably 1, 2, 3, 4 or 5, also 1, 2, or 3 substituents, attached at any available atom to produce a stable compound, wherein the substituents are selected from the group consisting of halogen, —NO₂, —CN, —OR^{a}, —SR^{a}, —OC(O)R^{a}, —OC(S)R^{a}, —C(O)R^{a}, —C(S)R^{a}, —C(O)OR^{a}, —C(S)OR^{a}, —S(O)R^{a}, —S(O)₂R^{a}, —C(O)NR^{a}R^{a}, —C(S)NR^{a}R^{a}, —S(O)₂NR^{a}R^{a}, —C(NH)NR^{b}R^{c}, —NR^{a}C(O)R^{a}, —NR^{a}C(S)R^{a}, —NR^{a}S(O)₂R^{a}, —NR^{a}C(O)NR^{a}R^{a}, —NR^{a}C(S)NR^{a}R^{a}, —NR^{a}S(O)₂NR^{a}R^{a}, —NR^{a}R^{a}, —R^{d}, —R^{e}, and —R^{f}. It is understood that with any substitution of aryl, heteroaryl, cycloalkyl, and heterocycloalkyl, including, for example, selection of R³ of paragraph [0028], selected substituents, including any combinations thereof, are chemically feasible and provide a stable compound.

The variables as used in the description of optional substituents for lower alkyl, lower alkenyl, lower alkynyl, cycloalkyl, heterocycloalkyl, aryl and heteroaryl are defined as follows:
—R^{a}, —R^{b}, and —R^{c} at each occurrence are independently selected from the group consisting of hydrogen, —R^{d}, —R^{e}, and —R^{f}, provided, however, that R^{a} bound to S of any SR^{a}, S(O)R^{a}, or S(O)₂R^{a}, or C of any C(S)R^{a} or C(O)R^{a} is not hydrogen, or
—R^{b} and —R^{c} combine with the nitrogen to which they are attached to form a 5-7 membered heterocycloalkyl or a 5 or 7 membered nitrogen containing heteroaryl, wherein the 5-7 membered heterocycloalkyl or 5 or 7 membered nitrogen containing heteroaryl are optionally substituted with one or more, preferably 1, 2, 3, 4 or 5, also 1, 2, or 3 substituents selected from the group consisting of halogen, cycloalkylamino, —NO₂, —CN, —OR^{k}, —SR^{k}, —NR^{k}R^{k}, —R^{m}, and —R^{o};
—R^{d} at each occurrence is independently lower alkyl optionally substituted with one or more, preferably 1, 2, 3, 4 or 5, also 1, 2 or 3 substituents selected from the group consisting of fluoro, —OR^{g}, —SR^{g}, —NR^{g}R^{g}, —C(O)R^{g}, —C(S)R^{g}, —S(O)R^{g}, —S(O)₂R^{g}, —OC(O)R^{g}, —OC(S)R^{g}, —C(O)OR^{g}, —C(S)OR^{g}, —C(O)NR^{g}R^{g}, —C(S)NR^{g}R^{g}, —S(O)₂NR^{g}R^{g}, —NR^{g}C(O)R⁸, —NR^{g}C(S)R^{g}, —NR^{g}S(O)₂R^{g}, —NR^{g}C(O)NR^{g}R^{g}, —NR^{g}C(S)NR^{g}, —NR^{g}S(O)₂NR^{g}R^{g}, and —R^{f};
—R^{e} at each occurrence is independently selected from the group consisting of lower alkenyl and lower alkynyl, wherein lower alkenyl or lower alkynyl are optionally substituted with one or more, preferably 1, 2, 3, 4 or 5, also 1, 2 or 3 substituents selected from the group consisting of fluoro, —OR^{g}, —SR⁹, —NR^{g}R^{g}, —C(O)R^{g}, —C(S)R^{g}, —S(O)R^{g}, —S(O)₂R^{g}, —OC(O)R^{g}, —OC(S)R^{g}, —C(O)OR^{g}, —C(S)OR^{g}, —C(O)NR^{g}, —C(S)NR^{g}R^{g}, —S(O)₂NR^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, —R^{d}, and —R^{f};
—R^{f} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2 or 3 substituents selected from the group consisting of halogen, —NO₂, —CN, —OR⁹, —SR⁹, —NR^{g}R^{g}, —C(O)R^{g}, —C(S)R^{g}, —S(O)R^{g}, —S(O)₂R^{g}, —OC(O)R^{g}, —OC(S)R^{g}, —C(O)OR^{g}, —C(S)OR^{g}, —C(O)NR^{g}R^{g}, —C(S)NR^{g}R^{g}, —S(O)₂NR^{g}R^{g}, —NR^{g}C(O)R^{g}, —NR^{g}C(S)R^{g}, —NR^{g}S(O)₂R^{g}, —NR^{g}C(O)NR^{g}R^{g}, —NR^{g}C(S)NR^{g}R^{g}, —NR^{g}S(O)₂NR^{g}R^{g}, —R^{m}—, and —R^{o};
—R^{g} at each occurrence is independently selected from the group consisting of hydrogen, —R^{h}, —Rⁱ, and —R^{j}, provided, however, that R^{g} bound to S of any SR^{g}, S(O)R^{g}, or S(O)₂R^{g}, or C of any C(S)R^{g} or C(O)R^{g} is not hydrogen;
—R^{h} at each occurrence is independently lower alkyl optionally substituted with one or more, preferably 1, 2, 3, 4 or 5, also 1, 2, or 3 substituents selected from the group consisting of fluoro, —OR^{k}, —SR^{k}, —NR^{k}R^{k}, —C(O)R^{k}, —C(S)R^{k}, —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, and —R^{o}, provided, however, that any substitution on the lower alkyl carbon bound to any O, S, or N of any OR^{h}, SR^{h}, or NR^{h} is selected from the group consisting of fluoro and —R^{o};
—Rⁱ at each occurrence is independently selected from the group consisting of C₃₋₆ alkenyl and C₃₋₆ alkynyl, wherein C₃₋₆ alkenyl or C₃₋₆ alkynyl are optionally substituted with one or more, preferably 1, 2, 3, 4 or 5, also 1, 2, or 3 substituents selected from the group consisting of fluoro, —OR^{k}, —SR^{k}, —NR^{k}R^{k}, —C(O)R^{k}, —C(S)R^{k}, —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, —R^{m} and —R^{o}, provided, however, that any substitution on the alkenyl or alkynyl carbon bound to any O, S, or N of any ORⁱ, SRⁱ, or NRⁱ is selected from the group consisting of fluoro, —R^{m} and —R^{o};
R^{j} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, —NO₂, —CN, —OR^{k}, —SR^{k}, —NR^{k}R^{k}, —C(O)R^{k}, —C(S)R^{k}, —S(O)R^{k}, —S(O)₂R^{k}, —C(O)NR^{k}R^{k}, —C(S)NR^{k}R^{k}, —S(O)₂NR^{k}R^{k}, —NR^{k}C(O)R^{k}, —NR^{k}C(S)R^{k}, —NR^{k}S(O)₂R^{k}, —NR^{k}C(O)NR^{k}R^{k}, —NR^{k}C(S)NR^{k}R^{k}, —NR^{k}S(O)₂NR^{k}R^{k}, —R^{m}, and —R^{o};
   —R^{m} at each occurrence is independently selected from the group consisting of lower alkyl, lower alkenyl and lower alkynyl, wherein lower alkyl is optionally substituted with one or more, preferably 1, 2, 3, 4 or 5, also 1, 2, or 3 substituents selected from the group consisting of —R^{o}, fluoro, lower alkoxy, fluoro substituted lower alkoxy, lower alkylthio, fluoro substituted lower alkylthio, mono-alkylamino, di-alkylamino, and cycloalkylamino, and wherein lower alkenyl or lower alkynyl are optionally substituted with one or more, preferably 1, 2, 3, 4 or 5, also 1, 2, or 3 substituents selected from the group consisting of —R^{o}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
   R^{k} at each occurrence is independently selected from the group consisting of hydrogen, —Rⁿ, and —R^{o}, provided, however, that R^{k} bound to S of any SR^{k}, S(O)R^{k}, or S(O)₂R^{k}, or C of any C(S)R^{k} or C(O)R^{k} is not hydrogen;
   —Rⁿ at each occurrence is independently selected from the group consisting of lower alkyl, C₃₋₆ alkenyl and C₃₋₆ alkynyl, wherein lower alkyl is optionally substituted with one or more, preferably 1, 2, 3, 4 or 5, also 1, 2, or 3 substituents selected from the group consisting of —R^{o}, fluoro, lower alkoxy, fluoro substituted lower alkoxy, lower alkylthio, fluoro substituted lower alkylthio, mono-alkylamino, di-alkylamino, and cycloalkylamino, provided, however, that any substitution of the lower alkyl carbon bound to the O of ORⁿ, S of SRⁿ, or N of any NRⁿ is fluoro or —R^{o}, and wherein C₃₋₆ alkenyl or C₃₋₆ alkynyl are optionally substituted with one or more, preferably 1, 2, 3, 4 or 5, also 1, 2, or 3 substituents selected from the group consisting of —R^{o}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino, provided, however, that any substitution of the C₃₋₆ alkenyl or C₃₋₆ alkynyl carbon bound to the O of ORⁿ, S of SRⁿ, or N of any NRⁿ is fluoro, lower alkyl fluoro substituted lower alkyl, or —R^{o};
   —R^{o} at each occurrence is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino.

“Lower alkoxy” denotes the group —OR^{p}, where R^{p} is lower alkyl. “Optionally substituted lower alkoxy” denotes lower alkoxy in which R^{p} is optionally substituted lower alkyl. Preferably, substitution of lower alkoxy is with 1, 2, 3, 4, or 5 substituents, also 1, 2, or 3 substituents. For example “fluoro substituted lower alkoxy” denotes lower alkoxy in which the lower alkyl is substituted with one or more fluoro atoms, where preferably the lower alkoxy is substituted with 1, 2, 3, 4 or 5 fluoro atoms, also 1, 2, or 3 fluoro atoms. It is understood that substitutions on lower alkoxy are attached at any available atom to produce a stable compound, substitution of lower alkoxy is preferably such that O, S, or N (except where N is a heteroaryl ring atom), are not bound to the lower alkyl carbon bound to the lower alkoxy O. Further, where lower alkoxy is described as a substituent of another moiety, the lower alkoxy oxygen is preferably not bound to a carbon atom that is bound to an O, S, or N of the other moiety (except where N is a heteroaryl ring atom), or to an alkene or alkyne carbon of the other moiety.

“Aryloxy” denotes the group —OR^{q}, where R^{q} is aryl. “Optionally substituted aryloxy” denotes aryloxy in which R^{q} is optionally substituted aryl. “Heteroaryloxy” denotes the group —OR^{r}, where R^{r} is heteroaryl. “Optionally substituted heteroaryloxy” denotes heteroaryloxy in which R^{r} is optionally substituted heteroaryl.

“Lower alkylthio” denotes the group —SR^{s}, where R^{s} is lower alkyl. “Substituted lower alkylthio” denotes lower alkylthio in which R^{s} is optionally substituted lower alkyl. Preferably, substitution of lower alkylthio is with 1, 2, 3, 4, or 5 substituents, also 1, 2, or 3 substituents. For example “fluoro substituted lower alkylthio” denotes lower alkylthio in which the lower alkyl is substituted with one or more fluoro atoms, where preferably the lower alkylthio is substituted with 1, 2, 3, 4 or 5 fluoro atoms, also 1, 2, or 3 fluoro atoms. It is understood that substitutions on lower alkylthio are attached at any available atom to produce a stable compound, substitution of lower alkylthio is such that O, S, or N (except where N is a heteroaryl ring atom), are preferably not bound to the lower alkyl carbon bound to the lower alkylthio S. Further, where lower alkylthio is described as a substituent of another moiety, the lower alkylthio sulfur is preferably not bound to a carbon atom that is bound to an O, S, or N of the other moiety (except where N is a heteroaryl ring atom), or to an alkene or alkyne carbon of the other moiety.

“Amino” or “amine” denotes the group —NH₂. “Mono-alkylamino” denotes the group —NHR^{t} where R^{t} is lower alkyl. “Di-alkylamino” denotes the group —NR^{t}R^{u}, where R^{t} and R^{u} are independently lower alkyl. “Cycloalkylamino” denotes the group —NR^{v}R^{w}, where R^{v} and R^{w} combine with the nitrogen to form a 5-7 membered heterocycloalkyl, where the heterocycloalkyl may contain an additional heteroatom within the ring, such as O, N, or S, and may also be further substituted with lower alkyl. Examples of cycloalkylamino include, but are not limited to, piperidine, piperazine, 4-methylpiperazine, morpholine, and thiomorpholine. It is understood that when mono-alkylamino, di-alkylamino, or cycloalkylamino are substituents on other moieties that are attached at any available atom to produce a stable compound, the nitrogen of mono-alkylamino, di-alkylamino, or cycloalkylamino as substituents is preferably not bound to a carbon atom that is bound to an O, S, or N of the other moiety (except where N is a heteroaryl ring atom) or to an alkene or alkyne carbon of the other moiety.

As used herein in connection with PPAR modulating compound, binding compounds or ligands, the term “specific for PPAR” and terms of like import mean that a particular compound binds to a PPAR to a statistically greater extent than to other biomolecules that may be present in or originally isolated from a particular organism, e.g., at least 2, 3, 4, 5, 10, 20, 50, 100, or 1000-fold greater binding. Also, where biological activity other than binding is indicated, the term “specific for PPAR” indicates that a particular compound has greater biological activity associated with binding to a PPAR than to other biomolecules (e.g., at a level as indicated for binding specificity). Similarly, the specificity can be for a specific PPAR with respect to other PPARs that may be present in or originally isolated from a particular organism.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. In some cases, the specificity is with reference to a limited set of other biomolecules, e.g., in the case of PPARs, in some cases the reference may be other receptors, or for a particular PPAR, it may be other PPARs. In some embodiments, the greater specificity is at least 2, 3, 4, 5, 8, 10, 50, 100, 200, 400, 500, or 1000-fold greater specificity. In the context of ligands interacting with PPARs, the terms “activity on”, “activity toward,” and like terms mean that such ligands have EC₅₀ less than 10 μM, less than 1 μM, less than 100 nM, less than 50 nM, less than 20 nM, less than 10 nM, less than 5 nM, or less than 1 nM with respect to at least one PPAR as determined in a generally accepted PPAR activity assay.

The term “composition” or “pharmaceutical composition” refers to a formulation suitable for administration to an intended animal subject for therapeutic purposes. The formulation includes a therapeutically significant quantity (i.e. a therapeutically effective amount) of at least one active compound and at least one pharmaceutically acceptable carrier or excipient, which is prepared in a form adapted for administration to a subject. Thus, the preparation is “pharmaceutically acceptable”, indicating that it does not have properties that would cause a reasonably prudent medical practitioner to avoid administration of the material to a patient, taking into consideration the disease or conditions to be treated and the respective route of administration. In many cases, such a pharmaceutical composition is a sterile preparation, e.g. for injectibles.

The term “PPAR-mediated” disease or condition and like terms refer to a disease or condition in which the biological function of a PPAR affects the development and, or course of the disease or condition, and/or in which modulation of PPAR alters the development, course, and/or symptoms of the disease or condition. Similarly, the phrase “PPAR modulation provides a therapeutic benefit” indicates that modulation of the level of activity of PPAR in a subject indicates that such modulation reduces the severity and or duration of the disease, reduces the likelihood or delays the onset of the disease or condition, and/or causes an improvement in one or more symptoms of the disease or condition. In some cases the disease or condition may be mediated by any one or more of the PPAR isoforms, e.g., PPARγ, PPARα, PPARδ, PPARγ and PPARα, PPARγ and PPARδ, PPARα and PPARδ, or PPARγ, PPARα, and PPARδ. In some cases, modulation of any one or more of the PPAR isoforms, e.g., PPARγ, PPARα, PPARδ, PPARγ and PPARα, PPARγ and PPARδ, PPARα and PPARδ, or PPARγ, PPARα, and PPARδ provides a therapeutic benefit.

The term “therapeutically effective” or “effective amount” indicates that the materials or amount of material is effective to prevent, alleviate, or ameliorate one or more symptoms of a disease or medical condition, and/or to prolong the survival of the subject being treated.

The term “PPAR” refers to a peroxisome proliferator-activated receptor as recognized in the art. As indicated above, the PPAR family includes PPARα (also referred to as PPARa or PPARalpha), PPARδ (also referred to as PPARd or PPARdelta), and PPARγ (also referred to as PPARg or PPARgamma). Additional details regarding identification of the individual PPARs by their sequences can be found, for example, in US Patent Application Publication number US 2007/0072904, the disclosure of which is hereby incorporated by reference in its entirety.

As used herein in connection with the design or development of ligands, the term “bind” and “binding” and like terms refer to a non-convalent energetically favorable association between the specified molecules (i.e., the bound state has a lower free energy than the separated state, which can be measured calorimetrically). For binding to a target, the binding is at least selective, that is, the compound binds preferentially to a particular target or to members of a target family at a binding site, as compared to non-specific binding to unrelated proteins not having a similar binding site. For example, BSA is often used for evaluating or controlling for non-specific binding. In addition, for an association to be regarded as binding, the decrease in free energy going from a separated state to the bound state must be sufficient so that the association is detectable in a biochemical assay suitable for the molecules involved.

By “assaying” is meant the creation of experimental conditions and the gathering of data regarding a particular result of the experimental conditions. For example, enzymes can be assayed based on their ability to act upon a detectable substrate. Likewise, for example, a compound or ligand can be assayed based on its ability to bind to a particular target molecule or molecules and or to modulate an activity of a target molecule.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “clog P” is meant the calculated log P of a compound, “P” referring to the partition coefficient of the compound between a lipophilic and an aqueous phase, usually between octanol and water.

In the context of compounds binding to a target, the term “greater affinity” indicates that the compound binds more tightly than a reference compound, or than the same compound in a reference condition, i.e., with a lower dissociation constant. In some embodiments, the greater affinity is at least 2, 3, 4, 5, 8, 10, 50, 100, 200, 400, 500, 1000, or 10,000-fold greater affinity.

By binding with “moderate affinity” is meant binding with a K_{D} of from about 200 nM to about 1 μM under standard conditions. By “moderately high affinity” is meant binding at a K_{D} of from about 1 nM to about 200 nM. By binding at “high affinity” is meant binding at a K_{D} Of below about 1 nM under standard conditions. The standard conditions for binding are at pH 7.2 at 37° C. for one hour. For example, typical binding conditions in a volume of 100 μl/well would comprise a PPAR, a test compound, HEPES 50 mM buffer at pH 7.2, NaCl 15 mM, ATP 2 μM, and bovine serum albumin (1 μg/well), at 37° C. for one hour.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured. For PPAR agonists, activities can be determined as described in the Examples, or using other such assay methods known in the art.

By “protein” is meant a polymer of amino acids. The amino acids can be naturally or non-naturally occurring. Proteins can also contain modifications, such as being glycosylated, phosphorylated, or other common modifications.

By “protein family” is meant a classification of proteins based on structural and/or functional similarities. For example, kinases, phosphatases, proteases, and similar groupings of proteins are protein families. Proteins can be grouped into a protein family based on having one or more protein folds in common, a substantial similarity in shape among folds of the proteins, homology, or based on having a common function. In many cases, smaller families will be specified, e.g., the PPAR family.

By “specific biochemical effect” is meant a therapeutically significant biochemical change in a biological system causing a detectable result. This specific biochemical effect can be, for example, the inhibition or activation of an enzyme, the inhibition or activation of a protein that binds to a desired target, or similar types of changes in the body's biochemistry. The specific biochemical effect can cause alleviation of symptoms of a disease or condition or another desirable effect. The detectable result can also be detected through an intermediate step.

By “standard conditions” is meant conditions under which an assay is performed to obtain scientifically meaningful data. Standard conditions are dependent on the particular assay, and can be generally subjective. Normally the standard conditions of an assay will be those conditions that are optimal for obtaining useful data from the particular assay. The standard conditions will generally minimize background signal and maximize the signal sought to be detected.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667 .$$

In the context of this invention, by “target molecule” is meant a molecule that a compound, molecular scaffold, or ligand is being assayed for binding to. The target molecule has an activity that binding of the molecular scaffold or ligand to the target molecule will alter or change. The binding of the compound, scaffold, or ligand to the target molecule can preferably cause a specific biochemical effect when it occurs in a biological system. A “biological system” includes, but is not limited to, a living system such as a human, animal, plant, or insect. In most but not all cases, the target molecule will be a protein or nucleic acid molecule.

By “pharmacophore” is meant a representation of molecular features that are considered to be responsible for a desired activity, such as interacting or binding with a receptor. A pharmacophore can include 3-dimensional (hydrophobic groups, charged/ionizable groups, hydrogen bond donors/acceptors), 2D (substructures), and 1D (physical or biological) properties.

As used herein in connection with numerical values, the terms “approximately” and “about” mean ±10% of the indicated value.

### Applications of PPAR Agonists

The PPARs have been recognized as suitable targets for a number of different diseases and conditions. Some of those applications are described, for example, in US Patent Application Publication number US 2007/0072904, the disclosure of which is hereby incorporated by reference in its entirety. Additional applications are known and the present compounds can also be used for those diseases and conditions.

Thus, PPAR agonists, such as those described herein by Formulae I, Ia, Ib, Ic, Id, Ie, If, Ig, Ih, Ii, Ij, Ik, Im, In, Io, Ip, and Iq, can be used in the prophylaxis and/or therapeutic treatment of a variety of different diseases and conditions, such as weight disorders (e.g., including, but not limited to, obesity, overweight condition, bulimia, and anorexia nervosa), lipid disorders (e.g., including, but not limited to, hyperlipidemia, dyslipidemia (including associated diabetic dyslipidemia and mixed dyslipidemia), hypoalphalipoproteinemia, hypertriglyceridemia, hypercholesterolemia, and low HDL (high density lipoprotein)), metabolic disorders (e.g., including, but not limited to, Metabolic Syndrome, Type II diabetes mellitus, Type I diabetes, hyperinsulinemia, impaired glucose tolerance, insulin resistance, diabetic complication (e.g., including, but not limited to, neuropathy, nephropathy, retinopathy, diabetic foot ulcer, bladder dysfunction, bowel dysfunction, diaphragmatic dysfunction and cataracts)), cardiovascular disease (e.g., including, but not limited to, hypertension, coronary heart disease, heart failure, congestive heart failure, atherosclerosis, arteriosclerosis, stroke, cerebrovascular disease, myocardial infarction, and peripheral vascular disease), inflammatory diseases (e.g., including, but not limited to, autoimmune diseases (e.g., including, but not limited to, vitiligo, uveitis, optic neuritis, pemphigus foliaceus, pemphigoid, inclusion body myositis, polymyositis, dermatomyositis, scleroderma, Grave's disease, Hashimoto's disease, chronic graft versus host disease, ankylosing spondylitis, rheumatoid arthritis, inflammatory bowel disease (e.g. ulcerative colitis, Crohn's disease), systemic lupus erythematosis, Sjogren's Syndrome, and multiple sclerosis), diseases involving airway inflammation (e.g., including, but not limited to, asthma and chronic obstructive pulmonary disease), inflammation in other organs (e.g., including, but not limited to, polycystic kidney disease (PKD), polycystic ovary syndrome, pancreatitis, nephritis, and hepatitis), otitis, stomatitis, sinusitis, arteritis, temporal arteritis, giant cell arteritis, and polymyalgia rheumatica), skin disorders (e.g., including, but not limited to, epithelial hyperproliferative diseases (e.g., including, but not limited to, eczema and psoriasis), dermatitis (e.g., including, but not limited to, atopic dermatitis, contact dermatitis, allergic dermatitis and chronic dermatitis), and impaired wound healing)), neurodegenerative disorders (e.g., including, but not limited to, Alzheimer's disease, Parkinson's disease, amyotrophic lateral sclerosis, spinal cord injury, and demyelinating disease (e.g., including, but not limited to, acute disseminated encephalomyelitis and Guillain-Barre syndrome)), coagulation disorders (e.g., including, but not limited to, thrombosis), gastrointestinal disorders (e.g., including, but not limited to, gastroesophageal reflux, appendicitis, diverticulitis, gastrointestinal ulcers, ileus, motility disorders and infarction of the large or small intestine), genitourinary disorders (e.g., including, but not limited to, renal insufficiency, erectile dysfunction, urinary incontinence, and neurogenic bladder), ophthalmic disorders (e.g., including, but not limited to, ophthalmic inflammation, conjunctivitis, keratoconjunctivitis, corneal inflammation, dry eye syndrome, macular degeneration, and pathologic neovascularization), infections (e.g., including, but not limited to, lyme disease, HCV, HIV, and Helicobacter pylori) and inflammation associated with infections (e.g., including, but not limited to, encephalitis, meningitis), neuropathic or inflammatory pain, pain syndromes (e.g., including, but not limited to, chronic pain syndrome, fibromyalgia), infertility, and cancer (e.g., including, but not limited to, breast cancer and thyroid cancer).

### PPAR Active Compounds

As indicated in the Summary of the Invention, and in connection with applicable diseases and conditions, a number of different PPAR agonists have been identified. In addition, the present invention provides PPAR agonist compounds described by Formulae I, Ia, Ih, Ic, Id, Ie, If, Ig, Ih, Ii, Ij, Ik, Im, In, Io, Ip, or Iq as provided in the Summary of the Invention above.

The activity of the compounds can be assessed using methods known to those of skill in the art, including, for example, methods described in US Patent Application Publication number US 2007/0072904, the disclosure of which is hereby incorporated by reference in its entirety.

(c) Isomers, Prodrugs, and Active Metabolites

Compounds contemplated herein are described with reference to both generic formulae and specific compounds. In addition, the invention compounds may exist in a number of different forms or derivatives, all within the scope of the present invention. Alternative forms or derivatives, such as (a) Isomers, Prodrugs, and Active Metabolites (b) Tautomers, Stereoisomers, Regioisomers, and Solvated Forms (c) Prodrugs and Metabolites (d) Pharmaceutically acceptable salts (e) Pharmaceutically acceptable formulations and (f) Polymorphic forms, are described, for example, in US Patent Application Publication number US 2007/0072904, the disclosure of which is hereby incorporated by reference in its entirety.

### Administration

The methods and compounds will typically be used in therapy for human subjects. However, they may also be used to treat similar or identical indications in other animal subjects. In this context, the terms “subject”, “animal subject”, and the like refer to human and non-human vertebrates, e.g., mammals such as non-human primates, sports and commercial animals, e.g., bovines, equines, porcines, ovines, rodents, and pets e.g., canines and felines. A description of possible methods and routes of administration may be found, for example, in US Patent Application Publication number US 2007/0072904, the disclosure of which is hereby incorporated by reference in its entirety.

### EXAMPLES

Examples related to the present invention are described below. In most cases, alternative techniques can be used. The examples are intended to be illustrative and are not limiting or restrictive to the scope of the invention.

### Example 1

### General Synthesis of Compounds of Formula I

Synthesis of compounds of Formula I where L is —S(O)₂— can be achieved in four steps as described in Scheme I.

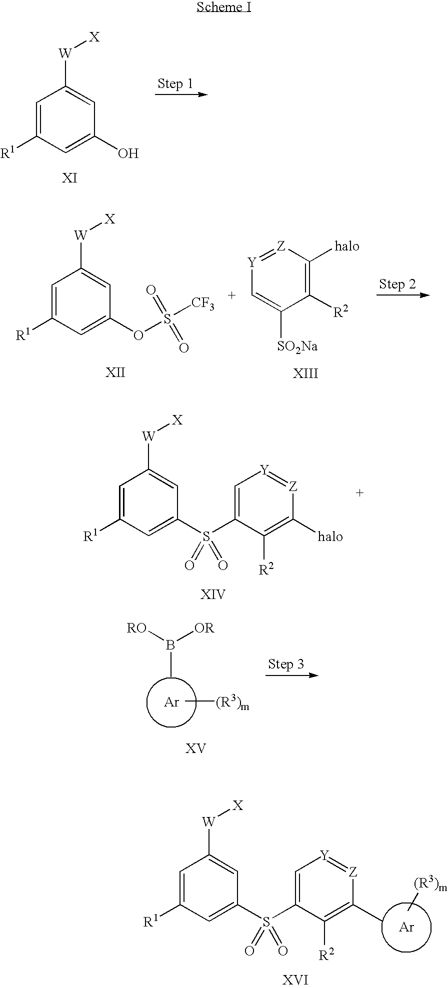

### Step 1—Preparation of Compound (XII)

Compound XII can be prepared via conversion of the hydroxyl group of compound XI (W, X as defined in paragraph [0028], R¹ is e.g. fluoro, chloro, optionally fluoro substituted methoxy, C₃₋₅ cycloalkyl, and C₁₋₃ alkyl, wherein C₁₋₃ alkyl is optionally substituted with one or more fluoro, methoxy, or fluoro substituted methoxy, see, for example XIa in following Step 1a) to a more labile group such as triflate through reaction with trifilic anhydride or tosyl sulfonyl chloride in an inert solvent such as pyridine.

### Step 1a —Preparation of Compound (XIa)

Compound XIa where R¹ is e.g. methoxy, optionally fluoro substituted methoxy, C₃₋₅ cycloalkyl, and C₁₋₃ alkyl, wherein C₁₋₃ alkyl is optionally substituted with one or more fluoro, methoxy, or fluoro substituted methoxy, for use in reaction Scheme I, can be prepared from compound X (W—X as defined in paragraph [0028], e.g. acetic acid methyl ester) via an alkylation reaction with an alkyl halide with a base such as potassium carbonate in an inert solvent such as 2-butanone, or via a Mitsunobu reaction with a hydroxyl group with triphenyl phosphine with an activation reagent such as diethylazodicarboxylate in an inert solvent such as tetrahydrofuran.

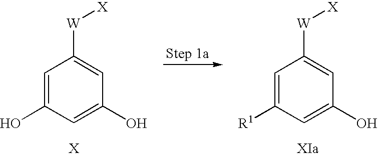

### Step 2—Preparation of Compound (XIV)

Compound XIV can be prepared by displacement of the triflate of XII with a sulfinic salt XIII (Y and Z are N or CH, R² is hydrogen, fluoro, chloro, C₁₋₃ alkyl or fluoro substituted C₁₋₃ alkyl, halo is iodo or bromo) through a catalyst such as palladium acetate, in a basic environment with an inert solvent such as toluene.

### Step 3—Preparation of Compound (XVI)

Compound XVI can be prepared through metal catalyzed (such as palladium) biaryl coupling of a boronic acid/ester XV (R³ as defined in paragraph [0028], m is 0-5, R is e.g. H) with the halogen substituted aromatic ring of XIV under basic conditions (i.e., Suzuki Cross Coupling, Muyaura and Suzuki, Chem. Rev. 1995, 95:2457). In the case where X is, for example, an alkyl ester, the compound XVI can be converted to the acid by deprotection of the alkyl ester through standard saponification conditions with a 1:1 ratio of an inert organic solvent, such as tetrahydrofuran and aqueous hydroxide solution (e.g., LiOH, NaOH, or KOH, 1 M) at ambient condition.

Alternatively, the fragment/substituent can be assembled before coupling to e.g. XII of Scheme I is outlined in Scheme II for compounds where XII is e.g. a phenyl acetic acid methyl ester.

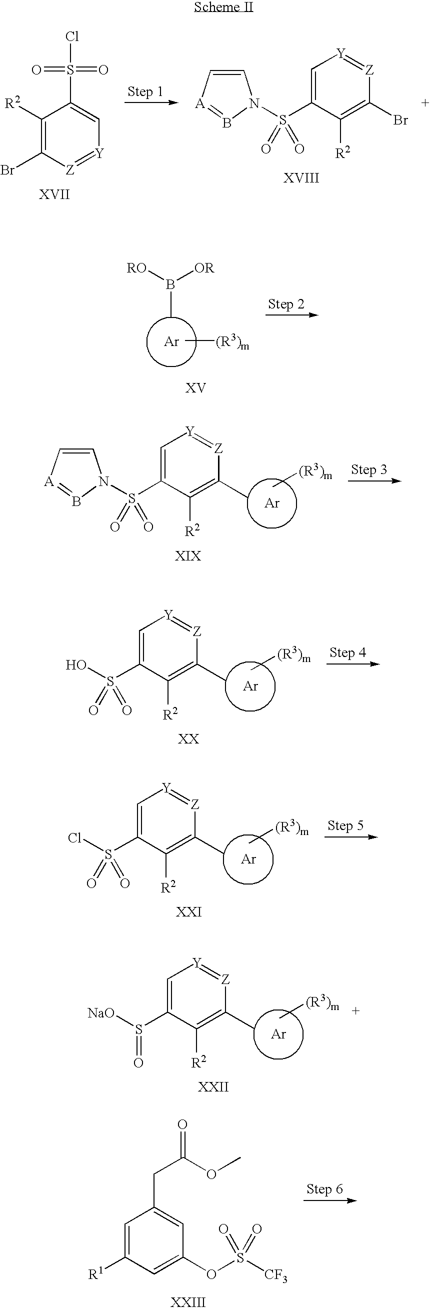

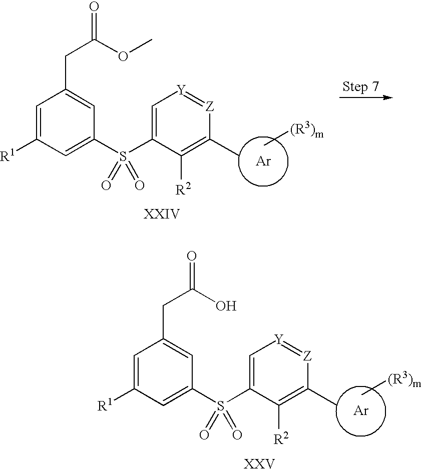

### Step 1—Preparation of Compound (XVIII)

Compound XVIII can be prepared through coupling of sulfonyl chloride XVII (Y and Z are N or CH, R² is hydrogen, fluoro, chloro, C₁₋₃ alkyl or fluoro substituted C₁₋₃ alkyl) with a heterocycle such as an imidazole or pyrrole (e.g. one of A or B is N, the other CH) in an inert solvent such as dichloromethane with a base such as triethylamine or N,N-dimethylaminopyridine.

### Step 2—Preparation of Compound (XIX)

Compound XIX can be prepared through metal catalyzed (such as palladium) biaryl coupling of a boronic acid/ester XV (R³ as defined in paragraph [0028], m is 0-5, R is e.g. H) with halogen (iodo or bromo) substituted aromatic ring of XVIII, under basic conditions (i.e., Suzuki Cross Coupling).

### Step 3—Preparation of Compound (XX)

Compound XX can be prepared through a basic hydrolysis of the sulfonamide XIX with the use of a base, such as potassium hydroxide in an inert solvent such as methanol with heating.

### Step 4—Preparation of Compound (XXI)

Compound XXI can be prepared through conversion of the acid functionality of XX with a reagent such as thionyl chloride or phosphorous pentachloride with a catalytic amount of N,N-dimethylformamide.

### Step 5—Preparation of Compound (XXII)

Compound XXII can be prepared through a reductive process of the corresponding sulfonyl chloride XXI with the use of a reagent such as sodium sulfite or zinc dust.

### Step 6—Preparation of Compound (XXIV)

Compound XXIV can be prepared by displacement of the triflate of XXIII (e.g. XII of Scheme I where W—X is acetic acid methyl ester, R¹ is e.g. fluoro, chloro, optionally fluoro substituted methoxy, C₃₋₅ cycloalkyl, and C₁₋₃ alkyl, wherein C₁₋₃ alkyl is optionally substituted with one or more fluoro, methoxy, or fluoro substituted methoxy) with a sulfinic salt XXII, through a catalyst such as palladium acetate, in a basic environment with an inert solvent such as toluene.

### Step 7—Preparation of Compound (XXV)

Compound XXV can be prepared through deprotection of the alkyl ester of XXIV through standard saponification conditions with a 1:1 ratio of an inert organic solvent, such as tetrahydrofuran and aqueous hydroxide solution (e.g., LiOH, NaOH, or KOH, 1M) at ambient condition.

Alternatively, a compound of Formula I where L is —O—, —S—, —S(O)— or —S(O)₂— (e.g. compound XXX) can be prepared as illustrated in Scheme III.

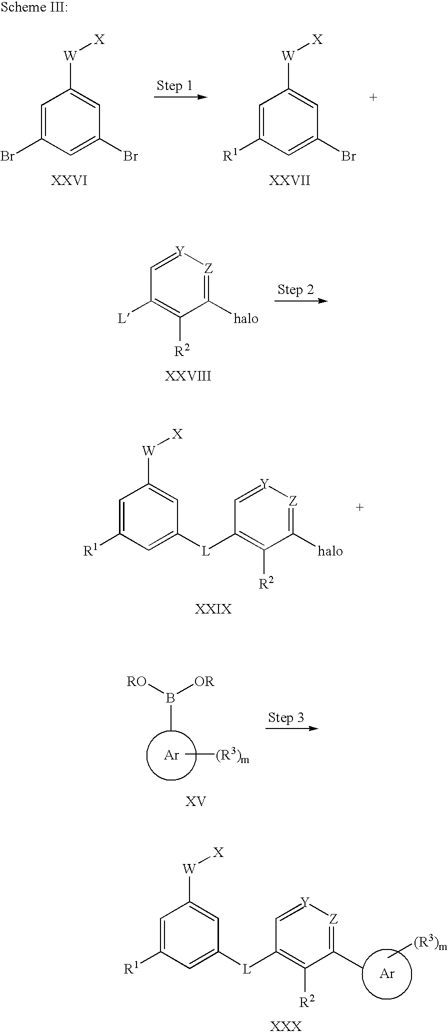

### Step 1—Preparation of Compound (XXVII)

Compound XXVII (where R¹ is methoxy or fluoro substituted methoxy) can be prepared via displacement of the bromide (or iodide) of compound XXVI (W, X as defined in paragraph [0028]) with a hydroxyl group (e.g. with optionally fluoro substituted methanol) with a catalyst such as palladium or copper in an inert solvent such as dimethyl formamide or dimethyl sulfoxide.

### Step 2—Preparation of Compound (XXIX)

Compound XXIX (where L is either O or S) can be prepared through displacement of the bromide (or iodide) of compound XXVII with a hydroxyl or thiol group (XXVIII, L′ is hydroxyl or thiol group, halo is e.g. bromo, chloro, iodo, Y and Z are N or CH, R² is hydrogen, fluoro, chloro, C₁₋₃ alkyl or fluoro substituted C₁₋₃ alkyl) with a catalyst such as palladium or copper in an inert solvent such as dimethyl formamide or dimethyl sulfoxide.

### Step 3—Preparation of Compound (XXX)

Compound XXX can be prepared through a Suzuki coupling of compound XXIX with a boronic acid/ester XV (R³ as defined in paragraph [0028], m is 0-5, R is e.g. H) with a palladium catalyst to generate a biaryl compound. Compounds where L is —S(O)— or —S(O)₂— can be prepared by selective oxidation of the thiol linker.

Alternatively, the fragment/substituent can be assembled before coupling to the phenyl acetic acid methyl ester core, as outlined in Scheme II above.

Synthesis of compounds of Formula I where W is —CH₂—, X is —COOH, R¹ is methoxy (shown with methoxy, could be mono, di or trifluoromethoxy), and L=—S(O)₂— (e.g. compound XXXIV) is presented in Scheme IV.

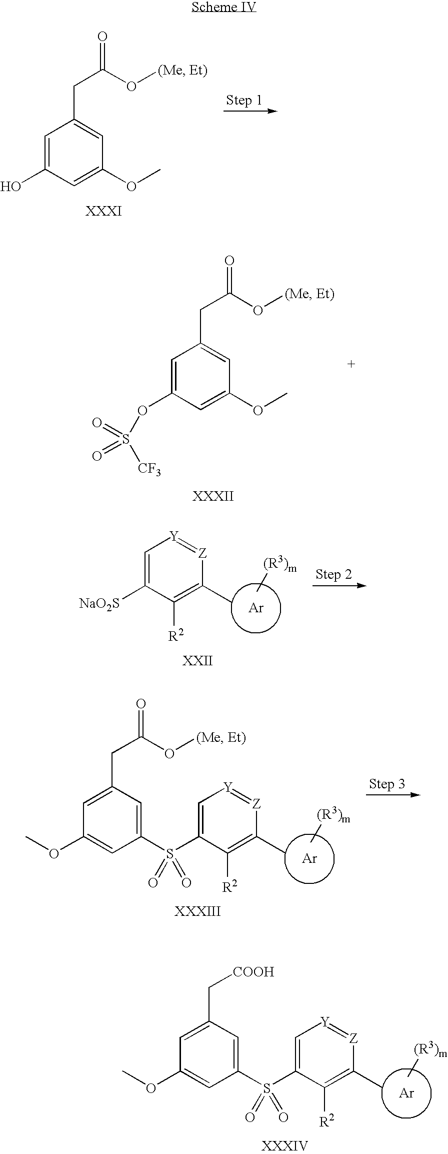

### Step 1—Preparation of Compound (XXXII)

Compound XXXII can be prepared through a generation of a “triflate” from reacting the hydroxy moiety in XXXI with trifluoromethylsulfonic anhydride in a buffered solvent such as pyridine.

### Step 2—Preparation of Compound (XXXIII)

Compound XXXIII can be prepared by displacement of the triflate of XXXII with a sulfinic salt (e.g. XXII per Scheme II above), through a catalyst such as palladium acetate, in a basic environment with an inert solvent such as toluene.

### Step 3—Preparation of Compound (XXXIV)

Compound XXXIV can be prepared by deprotection of the alkyl ester of XXXIII through standard saponification conditions with a 1:1 ratio of an inert organic solvent, such as tetrahydrofuran and aqueous hydroxide solution (e.g., LiOH, NaOH, or KOH, 1M) at ambient condition.

Synthesis of compounds of Formula I where W is —CH₂—, X is —COOH, R¹ is methoxy or fluoro substituted methoxy, and L=—S(O)₂— (e.g. compound XLII) is presented in Scheme V.

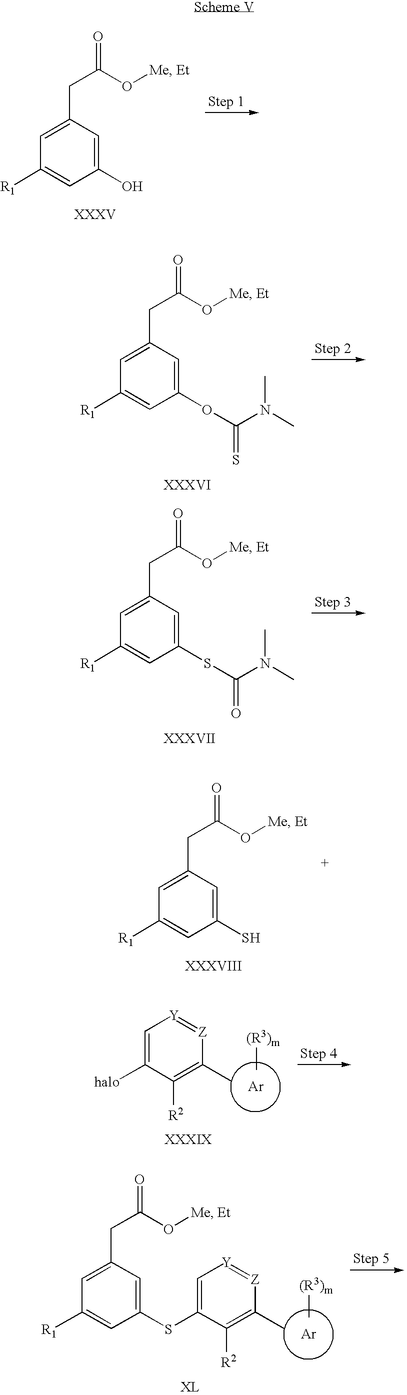

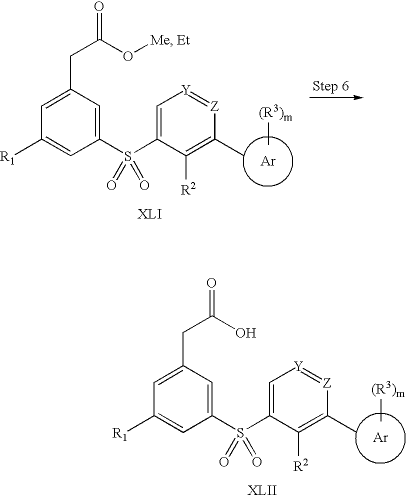

### Step 1—Preparation of Compound (XXXVI)

Compound XXXV (e.g. compounds XI, XIa of Scheme I where W—X is acetic acid methyl or ethyl ester) is treated with N,N-dimethylthiocarbamoyl chloride under basic environment in an inert solvent such as dimethyl formamide to provide compound XXXVI.

### Step 2—Preparation of Compound (XXXVII)

The thiocarbamate XXXVI is thermally rearranged to afford compound XXXVII, with the assistance of a microwave synthesizer, in an inert solvent such as dimethyl formamide or dimethyl sulfoxide.

### Step 3—Preparation of Compound (XXXVIII)

Compound XXXVIII can be prepared by hydrolysis of the thiocarbamate XXXVII under basic conditions (e.g., aqueous KOH) in an inert solvent such as methanol.

### Step 4—Preparation of Compound (XL)

Compound XL can be prepared through Ullman coupling conditions of the benzenethiol XXXVIII with a halogenated aromatic ring such as XXXIX (halo is bromo or iodo, Y and Z are N or CH, R² is hydrogen, fluoro, chloro, C₁₋₃ alkyl or fluoro substituted C₁₋₃ alkyl, R³ as defined in paragraph [0028], m is 0-5) with a catalyst such as cuprous iodide under basic environment in an inert solvent such as dioxane.

### Step 5—Preparation of Compound (XLI)

Biaryl thiol ether XL can be converted to the sulfone XLI through exposure to an oxidant such as m-chloroperbenzoic acid in an inert solvent such as dichloromethane.

### Step 6—Preparation of Compound (XLII)

Compound XLII can be prepared by deprotection of the alkyl ester of XLI under standard saponification conditions with a 1:1 ratio of an inert organic solvent, such as tetrahydrofuran and aqueous hydroxide solution (e.g., LiOH, NaOH, or KOH, 1M) at ambient condition.

Synthesis of compounds of Formula I where W is —OCH₂—, X is —COOH, R¹ is methoxy or fluoro substituted methoxy, and L=—S(O)₂— (e.g. compound XLVIII, shown with R¹ as methoxy, could also be fluoro substituted methoxy) is presented in Scheme VI.

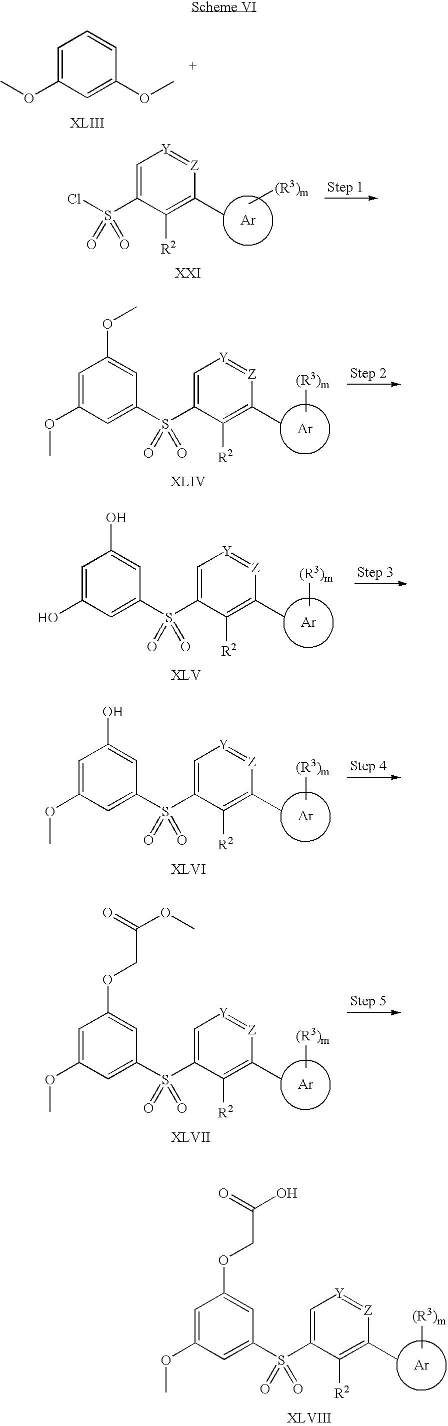

### Step 1—Preparation of Compound (XLIV)

Compound XLIV can be prepared through Friedel-Craft sulfonylation with a dimethoxybenzene XLIII and compound XXI (see Scheme II above) under acidic conditions such as indium trichloride.

### Step 2—Preparation of Compound (XLV)

Compound XLV can be prepared by de-methylation of XLIV with an acid, such as boron tribromide, at 0° C.

### Step 3—Preparation of Compound (XLVI)

Compound XLVI can be prepared by reacting XLV with an alkyl halide such as iodomethane (or fluoro substituted iodomethane) with a non-nucleophilic base such as potassium carbonate in an inert solvent such as dimethyl formamide with heating.

### Step 4—Preparation of Compound (XLVII)

Compound XLVII can be prepared by reaction of XLVI with a bromo acetic acid ester and a non-nucleophilic base such as potassium carbonate in an inert solvent such as dimethyl formamide with heating.

### Step 5—Preparation of Compound (XLVIII)

Compound XLVIII can be prepared by deprotection of the alkyl ester of XLVII under standard saponification conditions with a 1:1 ratio of an inert organic solvent, such as tetrahydrofuran and aqueous hydroxide solution (e.g., LiOH, NaOH, or KOH, 1M) at ambient condition.

Synthesis of compounds of Formula I where W is —CH₂—, X is —COOH, R¹═Cl or alkyl, and L=—S(O)₂— (e.g. compound LIII) is presented in Scheme VII.

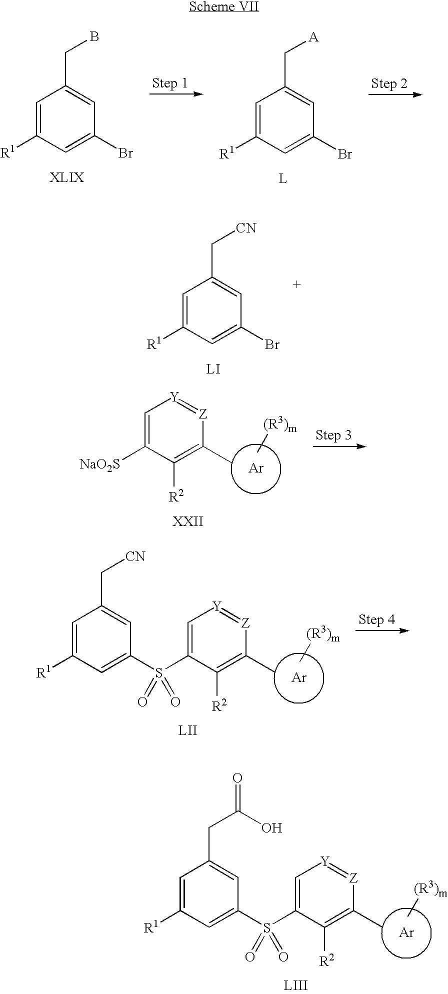

### Step 1—Preparation of Compound (L)

Compound XLIX (where B is H or OH and R¹ is Cl or alkyl) can be converted to compound L through halogenation when B═H, or conversion from B═OH to a halogen moiety A (e.g. chloro, bromo, iodo) through the use of reagents such as PCl₅ or PBr₃.

### Step 2—Preparation of Compound (LI)

Compound LI can be prepared through conversion of the halogen group of L to nitrile through the use of cyanide group in an inert solvent such as ethanol with heating.

### Step 3—Preparation of Compound (LII)

Compound LII can be prepared by displacement of the bromide of L with a sulfinic salt XXII (see Scheme II above), through a catalyst such as palladium acetate, in a basic environment with an inert solvent such as toluene.

### Step 4—Preparation of Compound (LIII)

Compound LIII can be prepared through hydrolysis of the nitrile group of LII through the use of hydroxide in an aqueous ethanol solution with heating.

Synthesis of compounds of Formula I where W is —CH₂—, X is —COOH and L=—NHS(O)₂— (e.g. compound LIX) is presented in Scheme VIII.

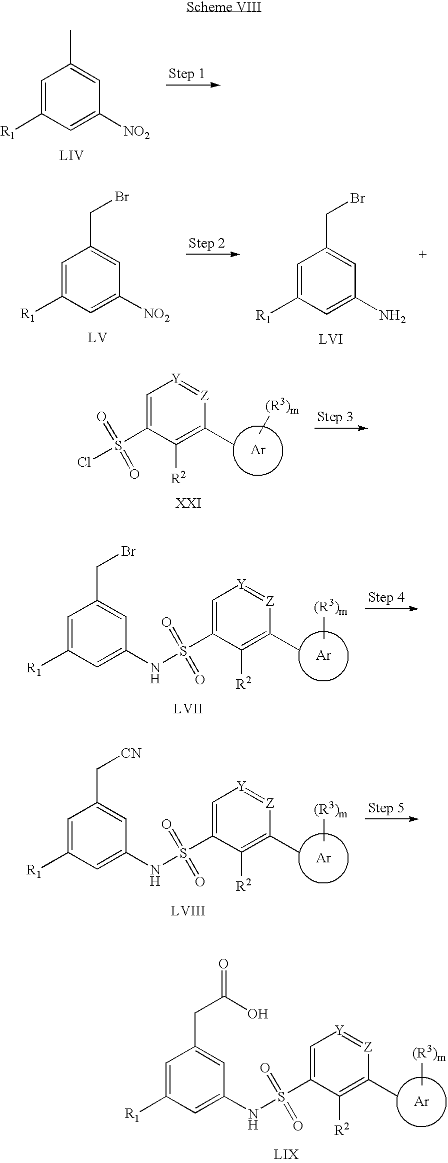

### Step 1—Preparation of Compound (LV)

Compound LV can be prepared from starting material LIV (R¹ is e.g. fluoro, chloro, optionally fluoro substituted methoxy, C₃₋₅ cycloalkyl, and C₁₋₃ alkyl, wherein C₁₋₃ alkyl is optionally substituted with one or more fluoro, methoxy, or fluoro substituted methoxy) using N-bromosuccinimide in an inert solvent such as carbon tetrachloride with benzoyl peroxide as a catalyst with heating.

### Step 2—Preparation of Compound (LVI)

Compound LVI can be prepared through reduction of the nitro group of LV with a heterogeneous catalyst such as palladium on activated carbon in an inert solvent such as methanol with hydrogen gas.

### Step 3—Preparation of Compound (LVII)

Compound LVII can be prepared through reacting the aniline group of LVI with a sulfonyl chloride XXI (see Scheme II above) in an inert solvent such as dichloromethane or pyridine.

### Step 4—Preparation of Compound (LVIII)

Compound LVIII can be prepared through conversion of the bromo of LVII to nitrile through the use of cyanide group in an inert solvent such as ethanol with heating.

### Step 5—Preparation of Compound (LIX)

Compound LIX can be prepared through hydrolysis of the nitrile group of LVIII through the use of hydroxide in an aqueous ethanol solution with heating.

### Example 2

### Synthesis of [3-(4′-chloro-biphenyl-3-sulfonyl)-5-methoxy-phenyl]-acetic acid P-0016

[3-(4′-Chloro-biphenyl-3-sulfonyl)-5-methoxy-phenyl]-acetic acid P-0016 was synthesized in four steps from (3,5-dihydroxy-phenyl)-acetic acid methyl ester 1 as shown in Scheme 1.

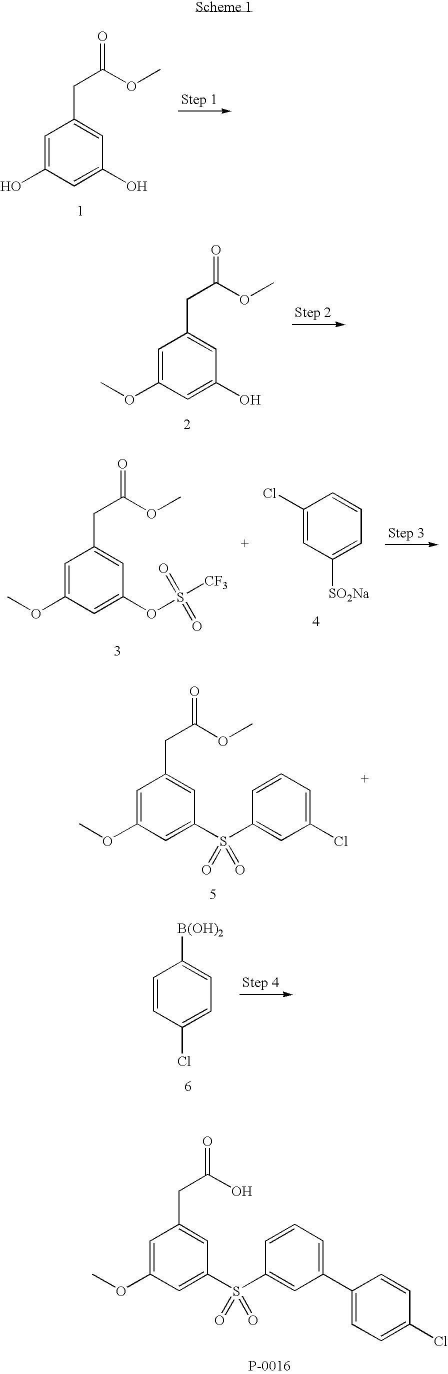

### Step 1—Preparation of (3-methoxy-5-hydroxy-phenyl)-acetic acid methyl ester (2)

Into a flask, (3,5-dihydroxy-phenyl)-acetic acid methyl ester (1, 4 g, 0.02 mol) was dissolved in 2-butanone (80 mL, 0.8 mol). Potassium carbonate (9.10 g, 0.0659 mol) was added in one portion and iodomethane (1.60 mL, 0.0200 mol) was added drop wise. The reaction was heated to 80° C. and left stirring for 5 hours. The solid was filtered off and the solvent was removed. Water and ethyl acetate were added, the solution was neutralized with 1M HCl, and the water phase was extracted with ethyl acetate. The pooled organic phase was dried (Na₂SO₄) and absorbed onto silica. Flash chromatography eluting with 20-40% ethyl acetate in hexanes afforded the desired compound as a clear yellow oil. ¹H NMR consistent with compound structure.

### Step 2—Preparation of (3-methoxy-5-trifluoromethanesulfonyloxy-phenyl)-acetic acid methyl ester (3)

Into a round bottom flask (3-methoxy-5-hydroxy-phenyl)-acetic acid methyl ester (2, 4 g, 0.02 mol) was dissolved in pyridine (60 mL, 0.7 mol) at 0° C. Trifluoromethanesulfonic anhydride (7 mL, 0.04 mol) was added in portions, and the reaction was left stirring for 16 hours and allowed to come to ambient conditions. The reaction was acidified with concentrated HCl and extracted 3× with diethyl ether. The combined organic layers were then washed 2× with brine, dried over sodium sulfate, and evaporated to yield a red-orange oil. The oil was then purified via flash chromatography with 20-35% ethyl acetate in hexane on silica to yield the desired compound as a yellow oil. ¹H NMR consistent with compound structure.

### Step 3—Preparation of [3-(3-chloro-benzenesulfonyl)-5-methoxy-phenyl]-acetic acid methyl ester (5)

Into a round bottom flask, (3-methoxy-5-trifluoromethanesulfonyloxy-phenyl)-acetic acid methyl ester (3, 1.26 g, 0.00384 mol), 3-chlorophenyl sulfinic acid sodium salt (4, 1.26 g, 0.00634 mol), toluene (30 mL, 0.3 mol), xanthphos (0.30 g, 0.00052 mol), tris(dibenzylideneacetone)dipalladium(0) (0.50 g, 0.00055 mol), and cesium carbonate (1.3 g, 0.0040 mol) were combined and heated at 108° C. for 16 hours. The reaction was allowed to cool to room temperature and diluted with water. The reaction was extracted 4× with ethyl acetate. The combined organic layers were washed 2× with water, 1× with brine, and dried over sodium sulfate. Evaporation of solvent led to a yellow-orange oil. The oil was then purified via flash chromatography (20-40% ethyl acetate in hexane) to yield the desired compound as a yellow oil. The oil was dissolved and treated for 16 hours before workup. The reaction was acidified with 10% HCl to pH 1-2 and extracted 4× with ethyl acetate. The combined organic layers were washed 1× with brine, and dried over sodium sulfate. Evaporation of solvent led to a yellow oil. The oil was then purified via flash chromatography at 9% methanol in dichloromethane to afford the desired compound as a lightly yellowish oil, which upon drying on high vac afforded a white solid. ¹H NMR consistent with compound structure.

### Step 4—Preparation of [3-(4′-chloro-biphenyl-3-sulfonyl)-5-methoxy-phenyl]-acetic acid (P-0016)

[3-(3-Chloro-benzenesulfonyl)-5-methoxy-phenyl]-acetic acid methyl ester (5, 10 mg) was dissolved in 400 μL of acetonitrile and 2 equivalents of 4-chlorophenyl boronic acid 6 was added. K₂CO₃ (1M, 200 μL) and Pd(AOc)₂/di-t-butylbiphenylphosphine (0.2M solution in toluene, 10 μL) were added to the reaction. The reaction mixture was heated for 10 minutes at 160° C. in the microwave. The solution was neutralized with acetic acid and the solvents removed under vacuum. The crude material was dissolved in 500 μL of dimethylsulfoxide and purified by HPLC eluting with a water/0.1% trifluoro acetic acid and acetonitrile/0.1% trifluoro acetic acid gradient, 20-100% acetonitrile over 16 minutes. Calculated molecular weight 416.04, MS(ESI) [M+H⁺]⁻=417.5.

The following compounds were prepared following the protocol of Scheme 1, optionally replacing the (3-methoxy-5-hydroxy-phenyl)-acetic acid methyl ester 2 with (3-hydroxy-phenyl)-acetic acid methyl ester in Step 2, and/or optionally replacing the 4-chlorophenyl boronic acid 6 with an appropriate boronic acid in Step 4:
{3-Methoxy-5-[3-(6-methoxy-pyridin-3-yl)-benzenesulfonyl]-phenyl}-acetic acid (P-0001),
{3-Methoxy-5-[3-(2-methoxy-pyrimidin-5-yl)-benzenesulfonyl]-phenyl}-acetic acid (P-0002),
(3-Methoxy-5-{3-[1-(3-methyl-butyl)-1H-pyrazol-4-yl]-benzenesulfonyl}-phenyl)-acetic acid (P-0003),
{3-[3-(1-Isobutyl-1H-pyrazol-4-yl)-benzenesulfonyl]-5-methoxy-phenyl}-acetic acid (P-0004),
{3-[3-(6-Methoxy-pyridin-3-yl)-benzenesulfonyl]-phenyl}-acetic acid (P-0005),
(3-{3-[1-(3-Methyl-butyl)-1H-pyrazol-4-yl]-benzenesulfonyl}-phenyl)-acetic acid (P-0006),
{3-[3-(1-Isobutyl-1H-pyrazol-4-yl)-benzenesulfonyl]-phenyl}-acetic acid (P-0001), (P-0007),
[3-(3′-Chloro-biphenyl-3-sulfonyl)-5-methoxy-phenyl]-acetic acid (P-0015),
[3-Methoxy-5-(4′-methoxy-biphenyl-3-sulfonyl)-phenyl]-acetic acid (P-0017),
[3-(4′-Fluoro-biphenyl-3-sulfonyl)-5-methoxy-phenyl]-acetic acid (P-0018),
[3-(3′-Chloro-4′-fluoro-biphenyl-3-sulfonyl)-5-methoxy-phenyl]-acetic acid (P-0019),
[3-(4′-Ethoxy-biphenyl-3-sulfonyl)-5-methoxy-phenyl]-acetic acid (P-0020),
[3-(3′-Fluoro-biphenyl-3-sulfonyl)-5-methoxy-phenyl]-acetic acid (P-0021),
[3-Methoxy-5-(3′-trifluoromethoxy-biphenyl-3-sulfonyl)-phenyl]-acetic acid (P-0022),
[3-Methoxy-5-(4′-trifluoromethoxy-biphenyl-3-sulfonyl)-phenyl]-acetic acid (P-0023),
[3-Methoxy-5-(3′-trifluoromethyl-biphenyl-3-sulfonyl)-phenyl]-acetic acid (P-0024),
[3-(3′-Fluoro-4′-methyl-biphenyl-3-sulfonyl)-5-methoxy-phenyl]-acetic acid (P-0025),
[3-(3′-Fluoro-4′-methoxy-biphenyl-3-sulfonyl)-5-methoxy-phenyl]-acetic acid (P-0026),
[3-(3′-Chloro-biphenyl-3-sulfonyl)-phenyl]-acetic acid (P-0027),
[3-(4′-Chloro-biphenyl-3-sulfonyl)-phenyl]-acetic acid (P-0028),
[3-(4′-Methoxy-biphenyl-3-sulfonyl)-phenyl]-acetic acid (P-0029),
[3-(4′-Fluoro-biphenyl-3-sulfonyl)-phenyl]-acetic acid (P-0030),
[3-(4′-Ethoxy-biphenyl-3-sulfonyl)-phenyl]-acetic acid (P-0031),
[3-(3′-Fluoro-biphenyl-3-sulfonyl)-phenyl]-acetic acid (P-0032),
[3-(3′-Trifluoromethoxy-biphenyl-3-sulfonyl)-phenyl]-acetic acid (P-0033),
[3-(4′-Trifluoromethoxy-biphenyl-3-sulfonyl)-phenyl]-acetic acid (P-0034),
[3-(3′-Trifluoromethyl-biphenyl-3-sulfonyl)-phenyl]-acetic acid (P-0035),
[3-(3′-Fluoro-4′-methyl-biphenyl-3-sulfonyl)-phenyl]-acetic acid (P-0036),
[3-(3′-Fluoro-4′-methoxy-biphenyl-3-sulfonyl)-phenyl]-acetic acid (P-0037),
[3-(2′-Fluoro-biphenyl-3-sulfonyl)-5-methoxy-phenyl]-acetic acid (P-0062),
[3-(2′-Fluoro-4′-methoxy-biphenyl-3-sulfonyl)-5-methoxy-phenyl]-acetic acid (P-0063),
[3-(2′-Fluoro-biphenyl-3-sulfonyl)-phenyl]-acetic acid (P-0064),
[3-(4′-Benzyloxy-2′-fluoro-biphenyl-3-sulfonyl)-phenyl]-acetic acid (P-0065),
[3-(2′-Fluoro-4′-methoxy-biphenyl-3-sulfonyl)-phenyl]-acetic acid (P-0066),
[3-(4′-Chloro-2′-fluoro-biphenyl-3-sulfonyl)-phenyl]-acetic acid (P-0067),
[3-(Biphenyl-3-sulfonyl)-phenyl]-acetic acid (P-0080),
[3-(4′-Fluoro-2′-methyl-biphenyl-3-sulfonyl)-phenyl]-acetic acid (P-0081),
[3-(2′,3′-Difluoro-biphenyl-3-sulfonyl)-phenyl]-acetic acid (P-0082),
[3-(4′-Chloro-2′-methyl-biphenyl-3-sulfonyl)-phenyl]-acetic acid (P-0083),
[3-(2′-Chloro-4′-ethoxy-biphenyl-3-sulfonyl)-phenyl]-acetic acid (P-0084),
[3-(4′-Ethoxy-2′-methyl-biphenyl-3-sulfonyl)-phenyl]-acetic acid (P-0085),
[3-(Biphenyl-3-sulfonyl)-5-methoxy-phenyl]-acetic acid (P-0086),
[3-(2′,3′-Difluoro-biphenyl-3-sulfonyl)-5-methoxy-phenyl]-acetic acid (P-0087), and
[3-(4′-Chloro-2′-methyl-biphenyl-3-sulfonyl)-5-methoxy-phenyl]-acetic acid (P-0088).
   The following Table 1 indicates the compound number in Column 1, the acetic acid methyl ester used in Step 2 in Column 2, the boronic acid used in Step 4 in Column 3, the resulting compound structure in Column 4, and the experimental mass in Column 5.

TABLE I

| | Step 2 | | | Measured |
|---|---|---|---|---|
| Compound | acetic acid | Step 4 | | MS(ESI) |
| number | methyl ester | boronic acid | Resulting Compound | [M + H⁺]⁺ |
| P-0001 | 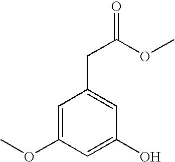 | 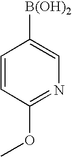 | 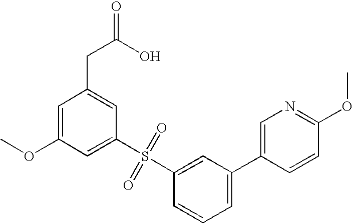 | 414.3 |
| P-0002 | 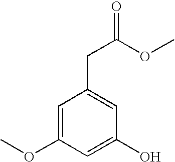 | 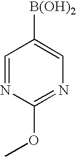 | 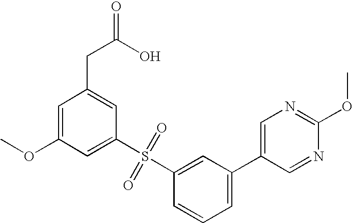 | 415.1 |
| P-0003 | 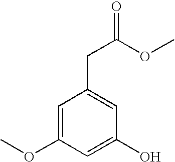 | 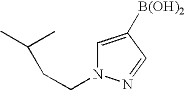 | 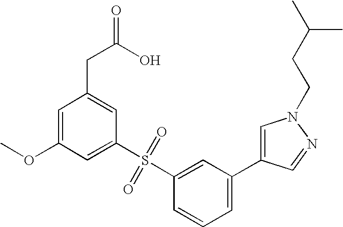 | 443.5 |
| P-0004 | 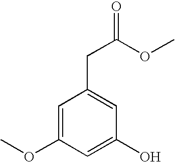 | 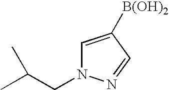 | 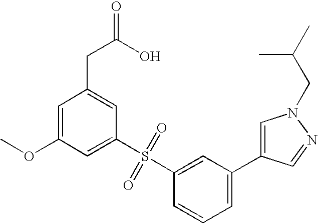 | 429.1 |
| P-0005 | 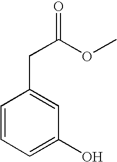 | 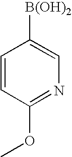 | 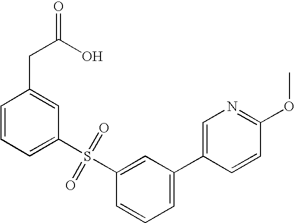 | 383.9 |
| P-0006 | 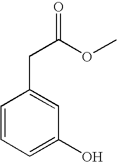 | 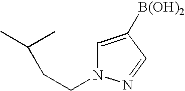 | 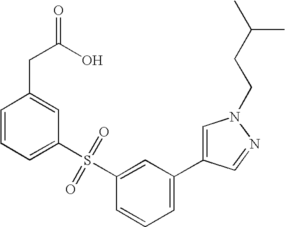 | 413.1 |
| P-0007 | 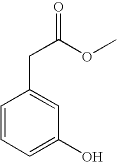 | 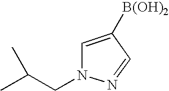 | 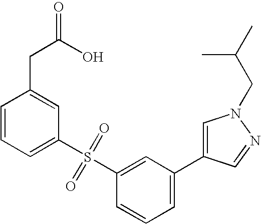 | 399.1 |
| P-0015 | 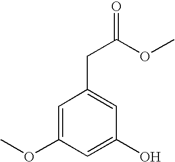 | 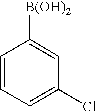 | 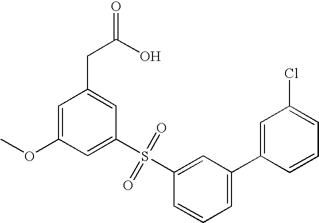 | 409.1 |
| P-0017 | 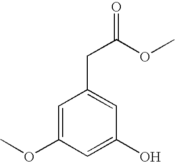 | 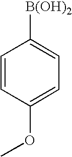 | 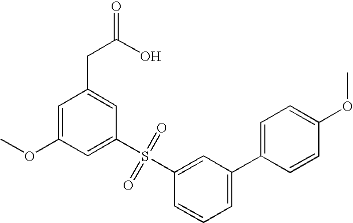 | 413.1 |
| P-0018 | 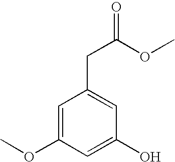 | 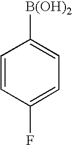 | 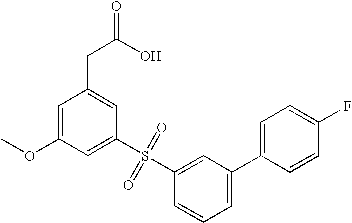 | 401.1 |
| P-0019 | 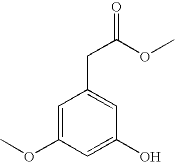 | 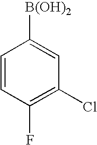 | 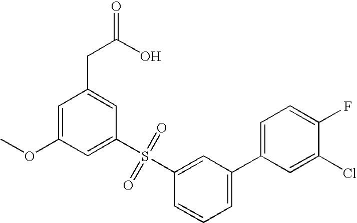 | 435.1 |
| P-0020 | 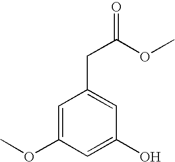 | 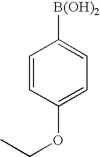 | 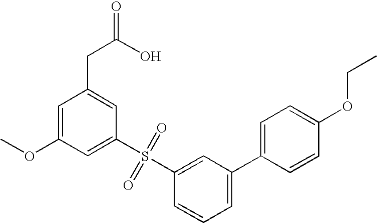 | 427.1 |
| P-0021 | 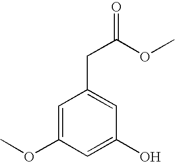 | 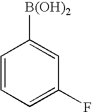 | 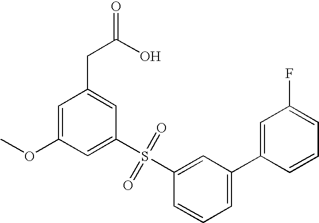 | 401.1 |
| P-0022 | 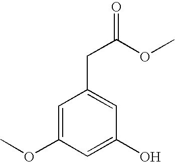 | 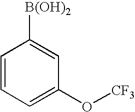 | 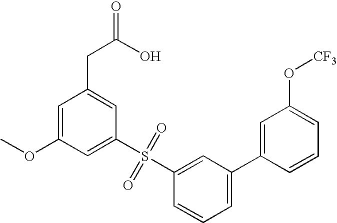 | 467.1 |
| P-0023 | 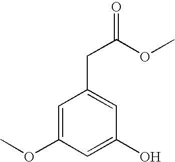 | 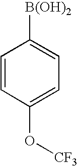 | 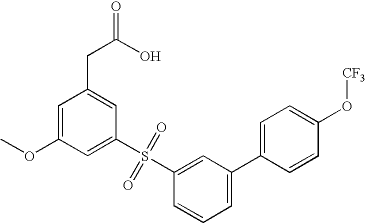 | 467.1 |
| P-0024 | 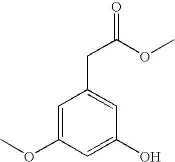 | 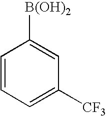 | 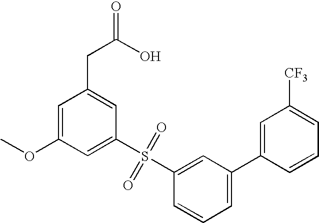 | 451.1 |
| P-0025 | 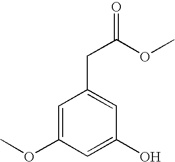 | 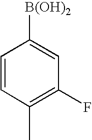 | 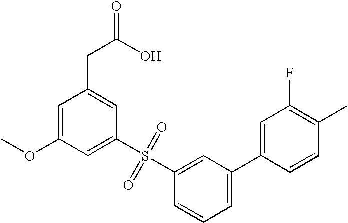 | 415.1 |
| P-0026 | 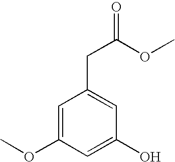 | 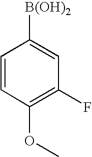 | 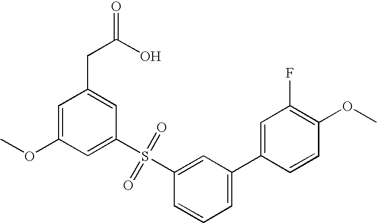 | 431.1 |
| P-0027 | 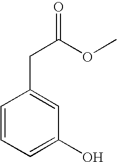 | 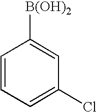 | 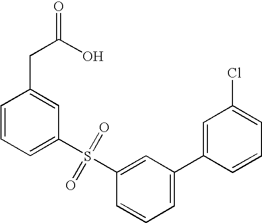 | 387.1 |
| P-0028 | 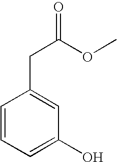 | 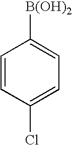 | 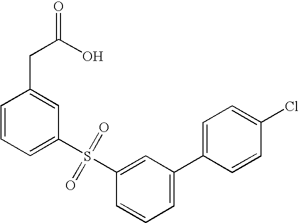 | 387.1 |
| P-0029 | 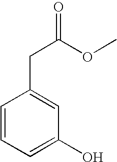 | 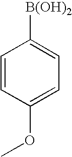 | 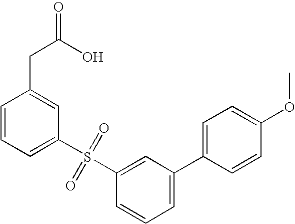 | 383.1 |
| P-0030 | 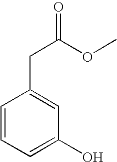 | 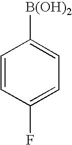 | 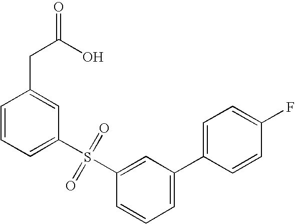 | 371.1 |
| P-0031 | 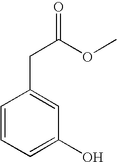 | 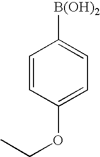 | 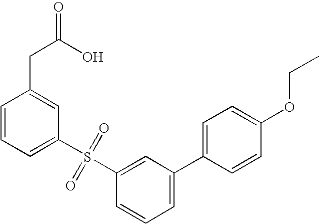 | 397.1 |
| P-0032 | 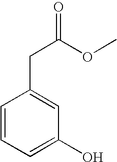 | 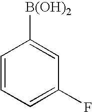 | 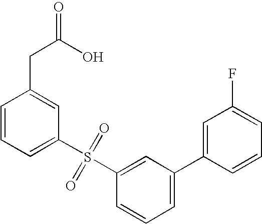 | 371.1 |
| P-0033 | 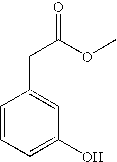 | 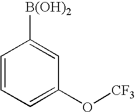 | 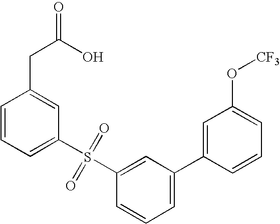 | 437.1 |
| P-0034 | 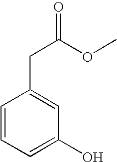 | 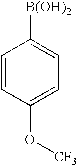 | 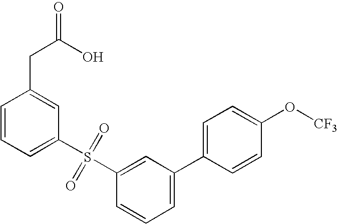 | 437.1 |
| P-0035 | 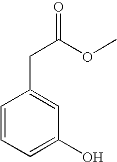 | 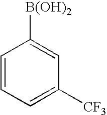 | 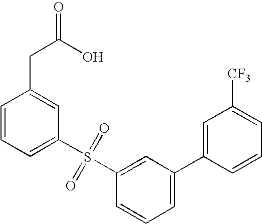 | 421.1 |
| P-0036 | 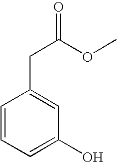 | 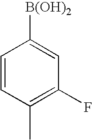 | 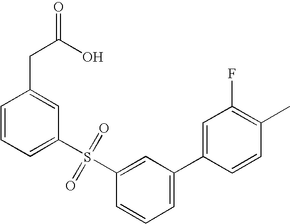 | 385.1 |
| P-0037 | 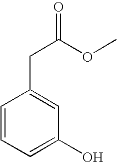 | 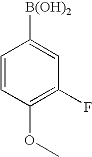 | 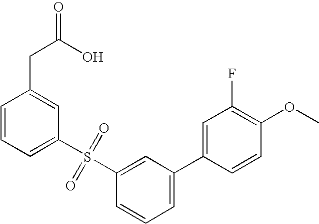 | 401.1 |
| P-0062 | 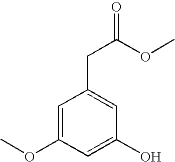 | 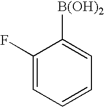 | 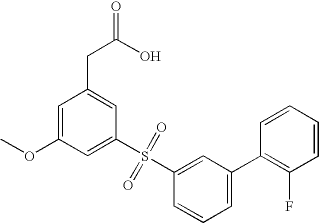 | 401.1 |
| P-0063 | 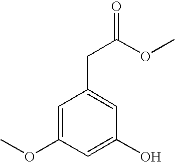 | 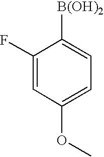 | 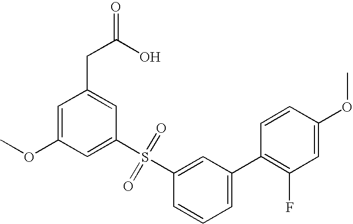 | 431.1 |
| P-0064 | 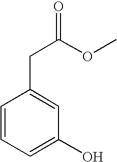 | 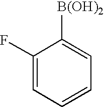 | 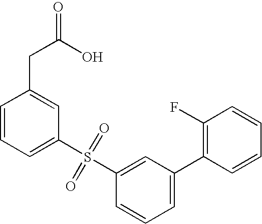 | 371.1 |
| P-0065 | 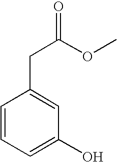 | 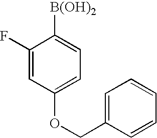 | 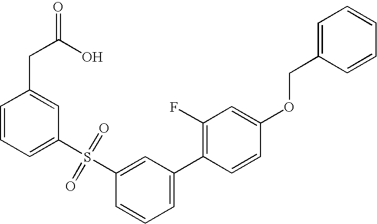 | 477.1 |
| P-0066 | 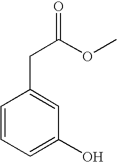 | 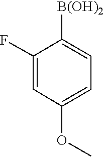 | 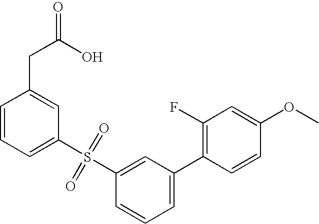 | 401.1 |
| P-0067 | 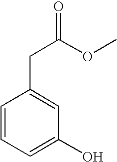 | 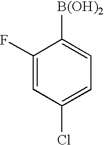 | 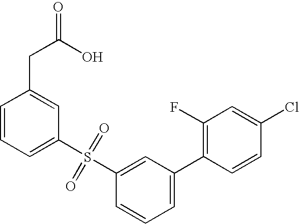 | 405.1 |
| P-0080 | 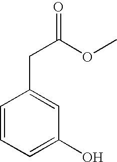 | 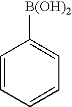 | 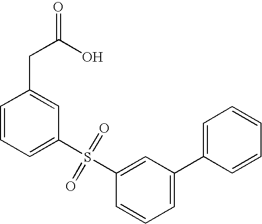 | 353.1 |
| P-0081 | 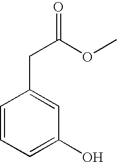 | 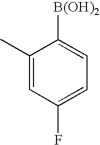 | 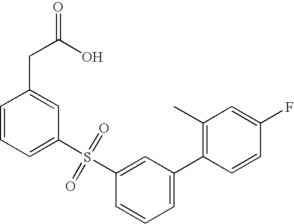 | 389.1 |
| P-0082 | 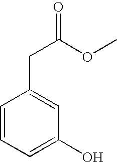 | 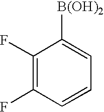 | 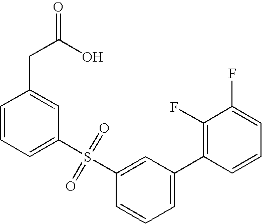 | 385.1 |
| P-0083 | 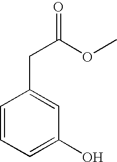 | 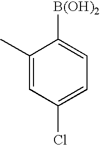 | 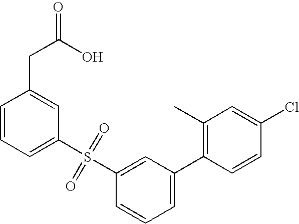 | 401.1 |
| P-0084 | 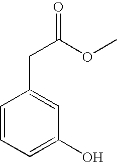 | 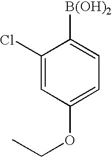 | 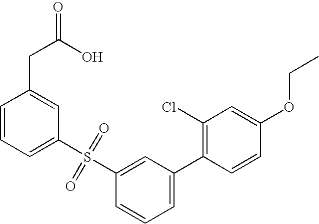 | 431.1 |
| P-0085 | 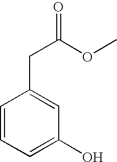 | 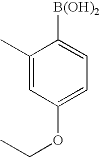 | 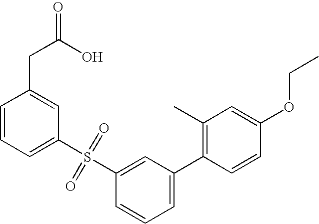 | 411.1 |
| P-0086 | 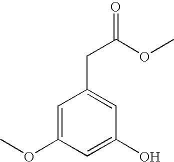 | 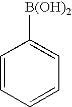 | 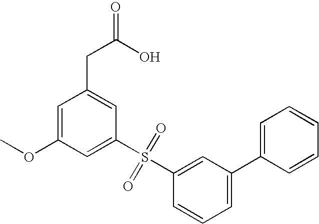 | 383.2 |
| P-0087 | 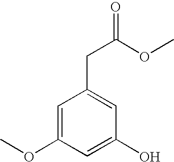 | 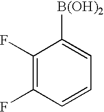 | 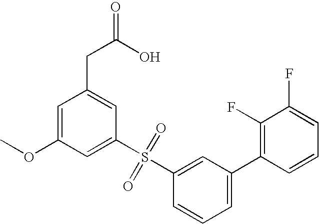 | 419.2 |
| P-0088 | 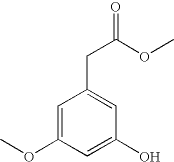 | 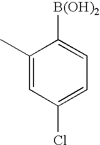 | 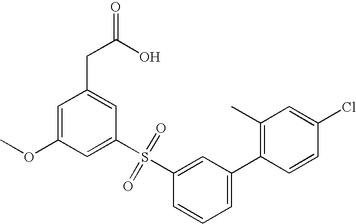 | 431.2 |

### Example 3

### Synthesis of [3-(4′-chloro-biphenyl-3-yloxy)-5-methoxy-phenyl]-acetic acid P-0039

[3-(4′-Chloro-biphenyl-3-yloxy)-5-methoxy-phenyl]-acetic acid P-0039 was synthesized in three steps from (3-methoxy-5-hydroxy-phenyl)-acetic acid methyl ester 2 as shown in Scheme 2.

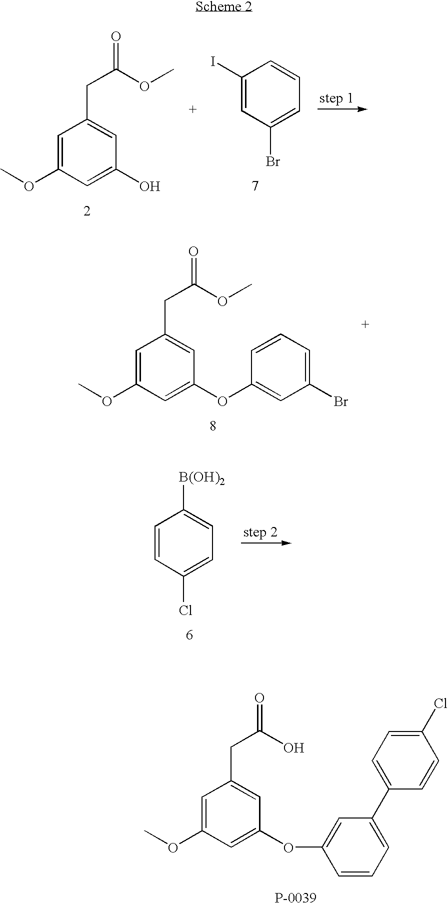

### Step 1—Preparation of [3-(3-bromo-phenoxy)-5-methoxy-phenyl]-acetic acid methyl ester (8)

To a solution of (3-methoxy-5-hydroxy-phenyl)-acetic acid methyl ester (2, 956 mg, 0.00487 mol, prepared as per Step 1 of Scheme 1, Example 2) dissolved in 1,4-dioxane (20 mL), cesium carbonate (3200 mg, 0.0097 mol), 1-bromo-3-iodo-benzene (7, 930 μL, 0.0073 mol), dimethylamino-acetic acid (200 mg, 0.001 mol) and copper(I) iodide (90 mg, 0.0005 mol) were added. The mixture was heated at 90° C. overnight under an atmosphere of argon. The reaction was diluted with a mixture of ammonium chloride:ammonium hydroxide 4:1 and extracted 3× with ethyl acetate. The combined organic layers were dried over sodium sulfate, concentrated under reduced pressure, and absorbed onto silica for flash chromatography. Using a gradient of 10-20% ethyl acetate in hexanes, the pure compound 8 was isolated. ¹H NMR consistent with compound structure.

### Step 2—Preparation of [3-(4′-chloro-biphenyl-3-yloxy)-5-methoxy-phenyl]-acetic acid (P-0039)

[3-(3-Bromo-phenoxy)-5-methoxy-phenyl]-acetic acid methyl ester (8, 10 mg, 0.03 mmol) was dissolved in 400 μL of acetonitrile and 4-chlorophenyl boronic acid (6, 5 mg, 0.05 mmol) was added. KC(C₃ (1M, 200 μl) was added and 10 μL of a 0.2 M solution of Pd(AOc)₂/di-tbutylbiphenylphosphine in toluene was added. The reaction mixture was irradiated for 10 min at 160° C. in a microwave synthesizer. The solution was neutralized with 50 μL of acetic acid and the solvents removed under reduced pressure. The crude material was dissolved in 500 μL of DMSO, plated and purified through reverse phase HPLC, using a YMC-Pack ODS-A C-18 column (50 mm×10 mm ID) at a gradient of 15%-80% B over 16 minutes. The mobile phase A was water with 0.1% TFA, and mobile phase B was acetonitrile with 0.1% TFA. Calculated molecular weight 368.81, MS(ESI) [M−H⁺]⁻=369.1.

The following compounds were prepared by optionally replacing the (3-methoxy-5-hydroxy-phenyl)-acetic acid methyl ester 2 with (3-hydroxy-phenyl)-acetic acid methyl ester in Step 1 and/or optionally replacing 4-chlorophenyl boronic acid with an appropriate boronic acid compound in Step 2:
{3-Methoxy-5-[3-(6-methoxy-pyridin-3-yl)-phenoxy]-phenyl}-acetic acid (P-0008),
{3-Methoxy-5-[3-(2-methoxy-pyrimidin-5-yl)-phenoxy]-phenyl}-acetic acid (P-0009),
{3-[3-(2,4-Dimethoxy-pyrimidin-5-yl)-phenoxy]-5-methoxy-phenyl}-acetic acid (P-0010),
(3-Methoxy-5-{3-[1-(3-methyl-butyl)-1H-pyrazol-4-yl]-phenoxy}-phenyl)-acetic acid (P-0011),
{3-[3-(1-Isobutyl-1H-pyrazol-4-yl)-phenoxy]-5-methoxy-phenyl}-acetic acid (P-0012),
{3-[3-(6-Methoxy-pyridin-3-yl)-phenoxy]-phenyl}-acetic acid (P-0013),
{3-[3-(2-Methoxy-pyrimidin-5-yl)-phenoxy]-phenyl}-acetic acid (P-0014),
[3-(3′-Chloro-biphenyl-3-yloxy)-5-methoxy-phenyl]-acetic acid (P-0038),
[3-Methoxy-5-(4′-methoxy-biphenyl-3-yloxy)-phenyl]-acetic acid (P-0040),
[3-(4′-Fluoro-biphenyl-3-yloxy)-5-methoxy-phenyl]-acetic acid (P-0041),
[3-(3′-Chloro-4′-fluoro-biphenyl-3-yloxy)-5-methoxy-phenyl]-acetic acid (P-0042),
[3-(4′-Ethoxy-biphenyl-3-yloxy)-5-methoxy-phenyl]-acetic acid (P-0043),
[3-(3′-Fluoro-biphenyl-3-yloxy)-5-methoxy-phenyl]-acetic acid (P-0044),
[3-Methoxy-5-(3′-trifluoromethoxy-biphenyl-3-yloxy)-phenyl]-acetic acid (P-0045),
[3-Methoxy-5-(4′-trifluoromethoxy-biphenyl-3-yloxy)-phenyl]-acetic acid (P-0046),
[3-Methoxy-5-(3′-trifluoromethyl-biphenyl-3-yloxy)-phenyl]-acetic acid (P-0047),
[3-(3′-Fluoro-4′-methyl-biphenyl-3-yloxy)-5-methoxy-phenyl]-acetic acid (P-0048),
[3-(3′-Fluoro-4′-methoxy-biphenyl-3-yloxy)-5-methoxy-phenyl]-acetic acid (P-0049),
[3-(3′-Chloro-biphenyl-3-yloxy)-phenyl]-acetic acid (P-0050),
[3-(4′-Chloro-biphenyl-3-yloxy)-phenyl]-acetic acid (P-0051),
[3-(4′-Methoxy-biphenyl-3-yloxy)-phenyl]-acetic acid (P-0052),
[3-(4′-Fluoro-biphenyl-3-yloxy)-phenyl]-acetic acid (P-0053),
[3-(3′-Chloro-4′-fluoro-biphenyl-3-yloxy)-phenyl]-acetic acid (P-0054),
[3-(4′-Ethoxy-biphenyl-3-yloxy)-phenyl]-acetic acid (P-0055),
[3-(3′-Fluoro-biphenyl-3-yloxy)-phenyl]-acetic acid (P-0056),
[3-(3′-Trifluoromethoxy-biphenyl-3-yloxy)-phenyl]-acetic acid (P-0057),
[3-(4′-Trifluoromethoxy-biphenyl-3-yloxy)-phenyl]-acetic acid (P-0058),
[3-(3′-Trifluoromethyl-biphenyl-3-yloxy)-phenyl]-acetic acid (P-0059),
[3-(3′-Fluoro-4′-methyl-biphenyl-3-yloxy)-phenyl]-acetic acid (P-0060),
[3-(3′-Fluoro-4′-methoxy-biphenyl-3-yloxy)-phenyl]-acetic acid (P-0061),
[3-(2′,4′-Difluoro-biphenyl-3-yloxy)-5-methoxy-phenyl]-acetic acid (P-0068),
[3-(2′-Fluoro-biphenyl-3-yloxy)-5-methoxy-phenyl]-acetic acid (P-0069),
[3-(4′-Benzyloxy-2′-fluoro-biphenyl-3-yloxy)-5-methoxy-phenyl]-acetic acid (P-0070),
[3-(4′-Chloro-2′-fluoro-biphenyl-3-yloxy)-5-methoxy-phenyl]-acetic acid (P-0071),
[3-(2′,4′-Difluoro-biphenyl-3-yloxy)-phenyl]-acetic acid (P-0072),
[3-(2′-Fluoro-biphenyl-3-yloxy)-phenyl]-acetic acid (P-0073),
[3-(4′-Benzyloxy-2′-fluoro-biphenyl-3-yloxy)-phenyl]-acetic acid (P-0074),
[3-(2′-Fluoro-4′-methoxy-biphenyl-3-yloxy)-phenyl]-acetic acid (P-0075),
[3-(4′-Chloro-2′-fluoro-biphenyl-3-yloxy)-phenyl]-acetic acid (P-0076),
{3-[3-(1-Isobutyl-1H-pyrazol-4-yl)-phenoxy]-phenyl}-acetic acid (P-0077),
[3-(2′-Fluoro-4′-trifluoromethyl-biphenyl-3-yloxy)-5-methoxy-phenyl]-acetic acid (P-0078),
[3-(2′-Fluoro-4′-trifluoromethyl-biphenyl-3-yloxy)-phenyl]-acetic acid (P-0079),
[3-(2′,5′-Dichloro-biphenyl-3-yloxy)-5-methoxy-phenyl]-acetic acid (P-0095),
[3-(2′,4′-Dichloro-biphenyl-3-yloxy)-5-methoxy-phenyl]-acetic acid (P-0096),
[3-(4′-Fluoro-2′-methyl-biphenyl-3-yloxy)-5-methoxy-phenyl]-acetic acid (P-0097),
[3-(3′,4′-Difluoro-biphenyl-3-yloxy)-5-methoxy-phenyl]-acetic acid (P-0098),
[3-(2′,3′-Dichloro-biphenyl-3-yloxy)-5-methoxy-phenyl]-acetic acid (P-0099),
[3-(4′-Fluoro-3′-methyl-biphenyl-3-yloxy)-5-methoxy-phenyl]-acetic acid (P-0100),
[3-(2′,3′-Difluoro-biphenyl-3-yloxy)-5-methoxy-phenyl]-acetic acid (P-0101),
[3-(2′-Fluoro-4′-methoxy-biphenyl-3-yloxy)-5-methoxy-phenyl]-acetic acid (P-0102),
[3-(4′-Fluoro-3′-trifluoromethyl-biphenyl-3-yloxy)-5-methoxy-phenyl]-acetic acid (P-0103),
[3-(4′-Chloro-2′-methyl-biphenyl-3-yloxy)-5-methoxy-phenyl]-acetic acid (P-0104),
[3-(2′-Chloro-4′-ethoxy-biphenyl-3-yloxy)-5-methoxy-phenyl]-acetic acid (P-0105),
[3-(2′-Chloro-4′-fluoro-biphenyl-3-yloxy)-5-methoxy-phenyl]-acetic acid (P-0106),
[3-(4′-Fluoro-2′-methyl-biphenyl-3-yloxy)-phenyl]-acetic acid (P-0107),
[3-(4′-Fluoro-3′-methyl-biphenyl-3-yloxy)-phenyl]-acetic acid (P-0108),
[3-(2′-Chloro-3′-fluoro-biphenyl-3-yloxy)-phenyl]-acetic acid (P-0109),
[3-(2′-Chloro-4′-trifluoromethyl-biphenyl-3-yloxy)-5-methoxy-phenyl]-acetic acid (P-0110),
[3-(5′-Chloro-2′-fluoro-biphenyl-3-yloxy)-5-methoxy-phenyl]-acetic acid (P-0111),
[3-(2′-Chloro-4′-trifluoromethyl-biphenyl-3-yloxy)-phenyl]-acetic acid (P-0112),
[3-(5′-Chloro-2′-fluoro-biphenyl-3-yloxy)-phenyl]-acetic acid (P-0113),
[3-(2′,5′-Dichloro-biphenyl-3-yloxy)-phenyl]-acetic acid (P-0114),
[3-(2′,4′-Dichloro-biphenyl-3-yloxy)-phenyl]-acetic acid (P-0115),
[3-(2′,3′-Dichloro-biphenyl-3-yloxy)-phenyl]-acetic acid (P-0116),
[3-(3′,4′-Dichloro-biphenyl-3-yloxy)-phenyl]-acetic acid (P-0117),
[3-(4′-Fluoro-3′-trifluoromethyl-biphenyl-3-yloxy)-phenyl]-acetic acid (P-0118),
[3-(4′-Chloro-2′-methyl-biphenyl-3-yloxy)-phenyl]-acetic acid (P-0119),
[3-(2′-Chloro-4′-fluoro-biphenyl-3-yloxy)-phenyl]-acetic acid (P-0120),
[3-(3′,4′-Difluoro-biphenyl-3-yloxy)-phenyl]-acetic acid (P-0121),
[3-(2′,3′-Difluoro-biphenyl-3-yloxy)-phenyl]-acetic acid (P-0122),
[3-(4′-Fluoro-3′-methoxy-biphenyl-3-yloxy)-phenyl]-acetic acid (P-0123),
[3-(2′-Chloro-4′-ethoxy-biphenyl-3-yloxy)-phenyl]-acetic acid (P-0124), and
[3-(4′-Ethoxy-2′-methyl-biphenyl-3-yloxy)-phenyl]-acetic acid (P-0125).
   The following Table 2 indicates the compound number in Column 1, the acetic acid methyl ester used in Step 1 in Column 2, the boronic acid used in Step 2 in Column 3, the resulting compound structure in Column 4, and the experimental mass in Column 5.

TABLE 2

| | Step 1 | | | Measured |
|---|---|---|---|---|
| Compound | acetic acid | Step 2 | | MS(ESI) |
| number | methyl ester | boronic acid | Resulting Compound | [M + H⁺]⁺ |
| P-0008 | 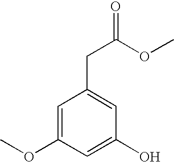 | 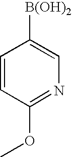 | 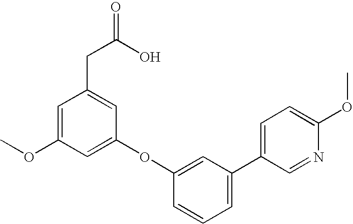 | 366.3 |
| P-0009 | 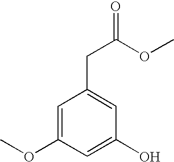 | 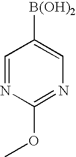 | 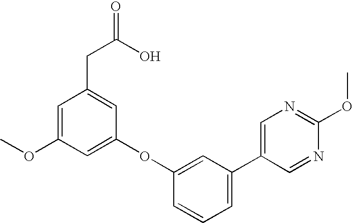 | 367.1 |
| P-0010 | 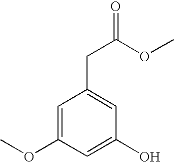 | 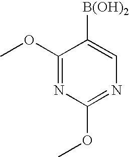 | 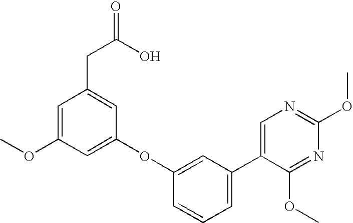 | 397.1 |
| P-0011 | 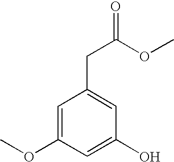 | 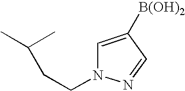 | 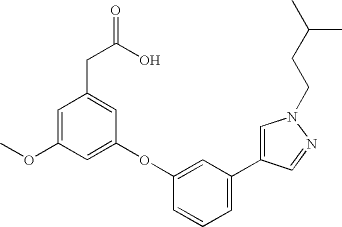 | 395.1 |
| P-0012 | 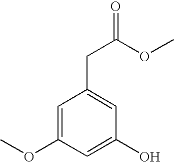 | 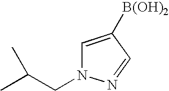 | 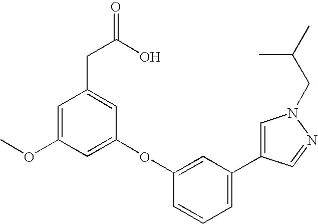 | 381.1 |
| P-0013 | 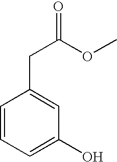 | 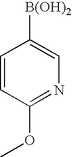 | 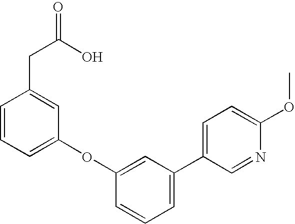 | 336.3 |
| P-0014 | 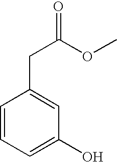 | 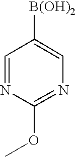 | 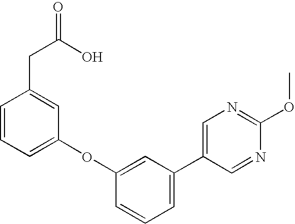 | 337.5 |
| P-0038 | 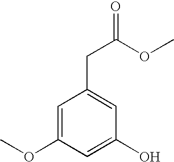 | 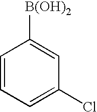 | 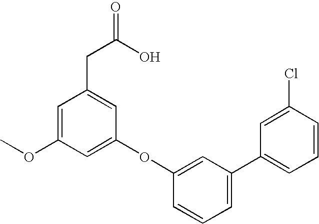 | 369.1 |
| P-0040 | 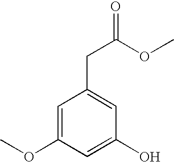 | 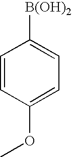 | 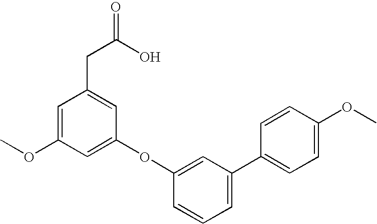 | 365.1 |
| P-0041 | 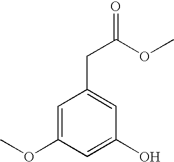 | 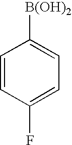 | 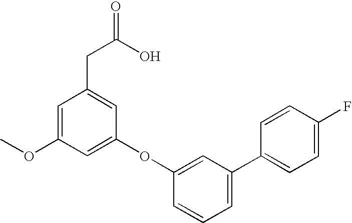 | 353.1 |
| P-0042 | 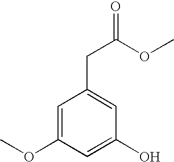 | 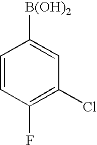 | 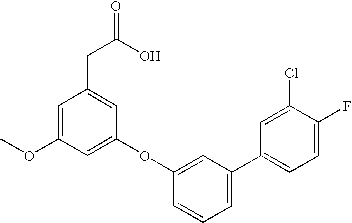 | 387.1 |
| P-0043 | 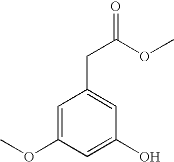 | 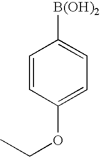 | 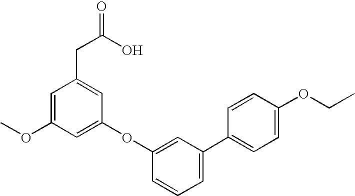 | 379.5 |
| P-0044 | 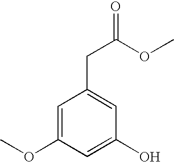 | 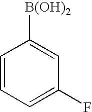 | 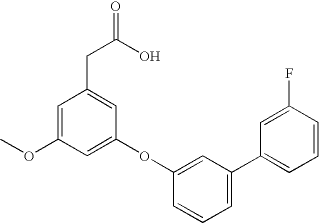 | 353.1 |
| P-0045 | 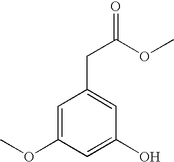 | 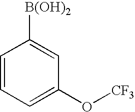 | 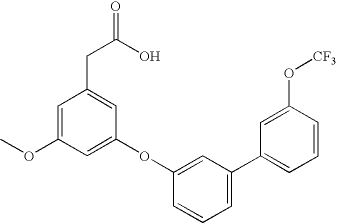 | 419.1 |
| P-0046 | 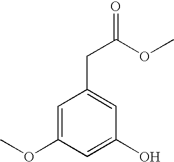 | 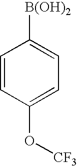 | 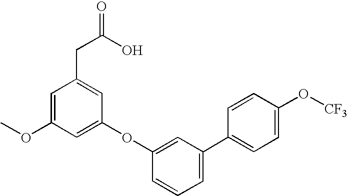 | 419.1 |
| P-0047 | 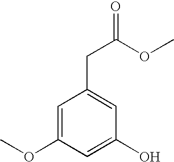 | 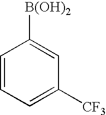 | 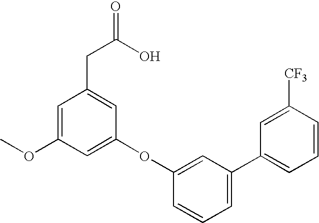 | 403.5 |
| P-0048 | 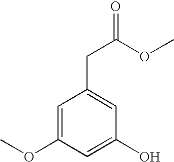 | 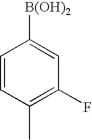 | 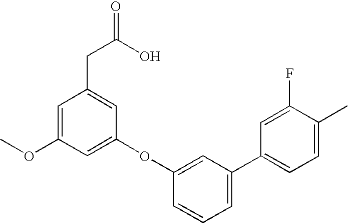 | 367.1 |
| P-0049 | 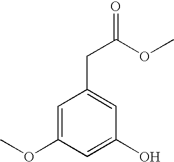 | 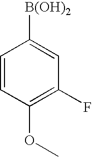 | 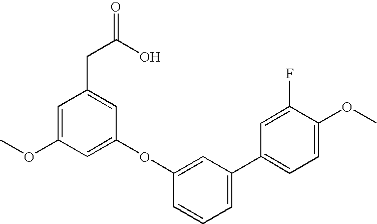 | 383.1 |
| P-0050 | 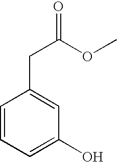 | 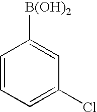 | 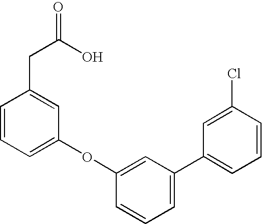 | 339.1 |
| P-0051 | 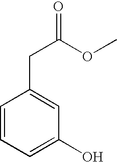 | 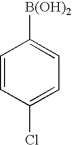 | 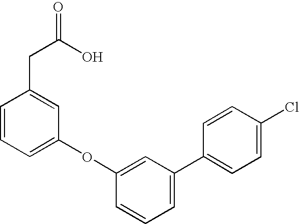 | 339.1 |
| P-0052 | 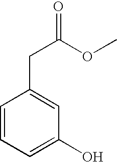 | 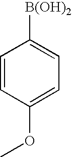 | 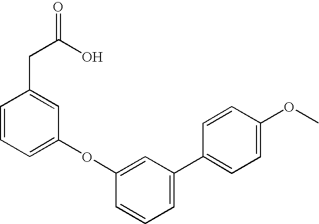 | 335.5 |
| P-0053 | 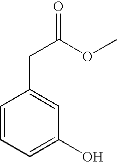 | 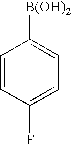 | 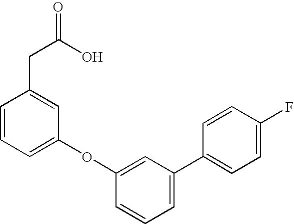 | 323.1 |
| P-0054 | 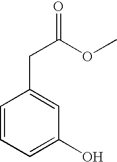 | 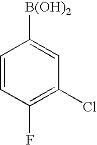 | 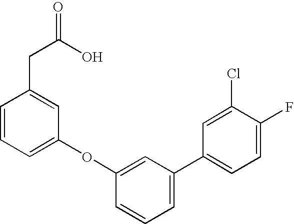 | 356.7 |
| P-0055 | 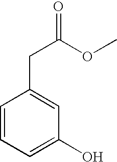 | 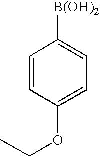 | 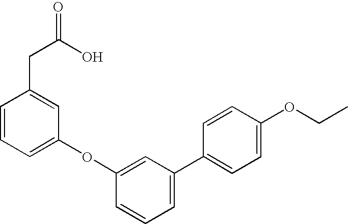 | 349.5 |
| P-0056 | 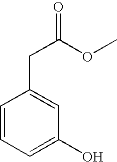 | 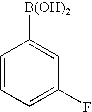 | 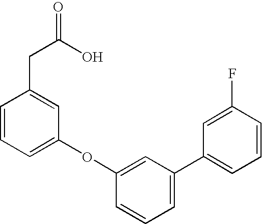 | 323.1 |
| P-0057 | 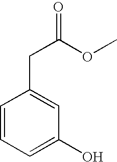 | 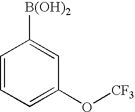 | 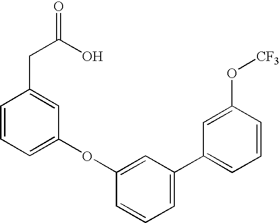 | 389.1 |
| P-0058 | 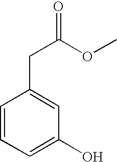 | 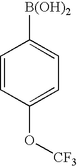 | 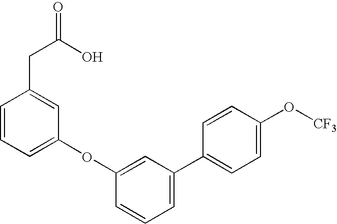 | 389.5 |
| P-0059 | 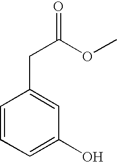 | 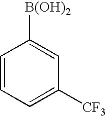 | 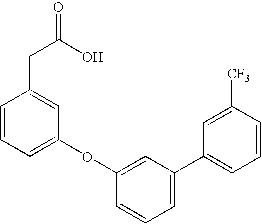 | 373.1 |
| P-0060 | 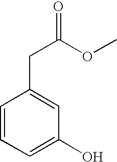 | 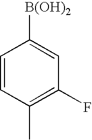 | 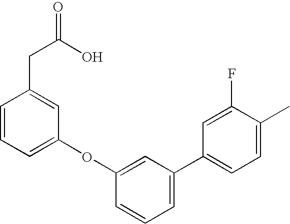 | 337.5 |
| P-0061 | 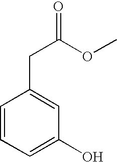 | 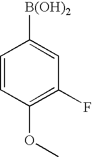 | 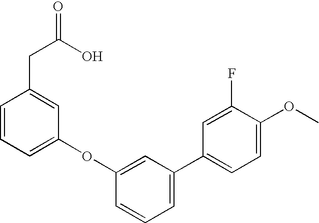 | 353.1 |
| P-0068 | 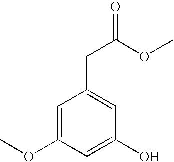 | 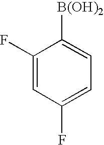 | 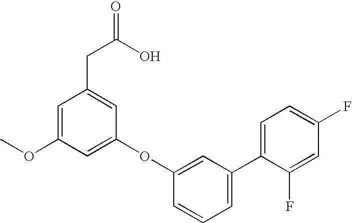 | 371.1 |
| P-0069 | 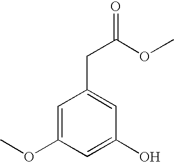 | 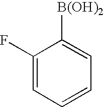 | 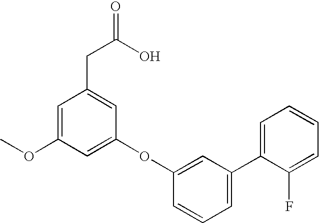 | 353.1 |
| P-0070 | 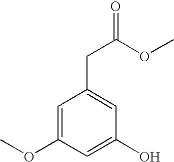 | 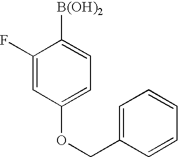 | 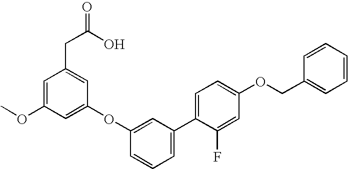 | 459.1 |
| P-0071 | 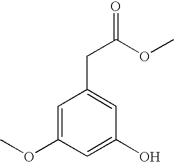 | 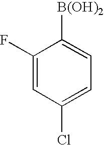 | 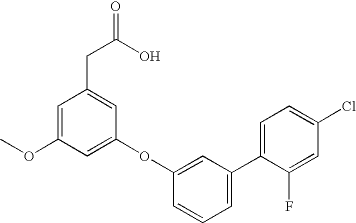 | 387.1 |
| P-0072 | 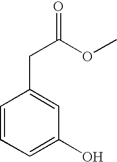 | 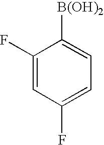 | 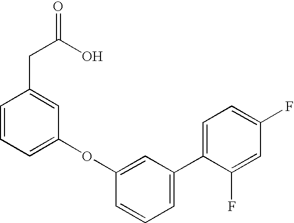 | 341.1 |
| P-0073 | 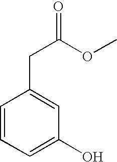 | 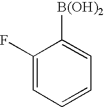 | 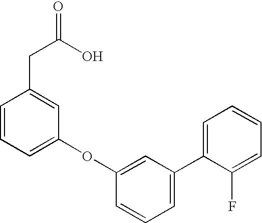 | 323.1 |
| P-0074 | 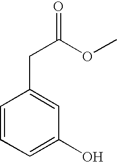 | 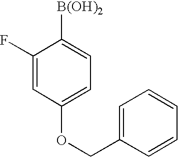 | 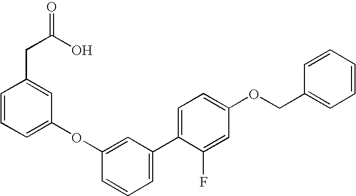 | 429.1 |
| P-0075 | 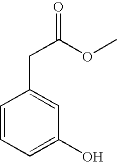 | 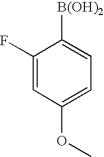 | 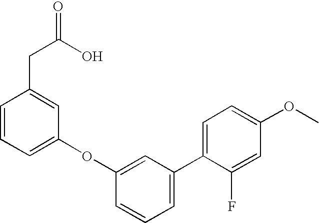 | 353.1 |
| P-0076 | 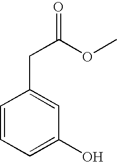 | 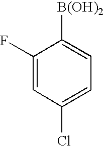 | 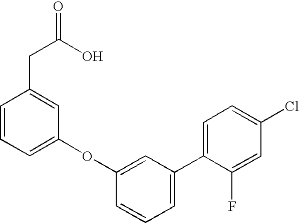 | 357.1 |
| P-0077 | 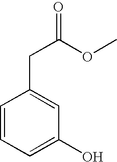 | 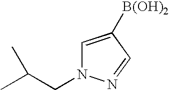 | 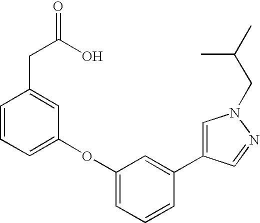 | 351.5 |
| P-0078 | 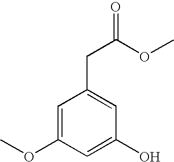 | 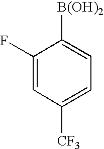 | 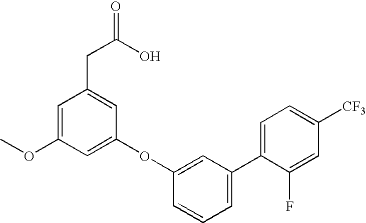 | 421.1 |
| P-0079 | 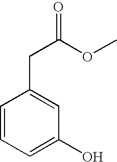 | 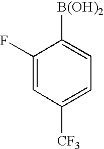 | 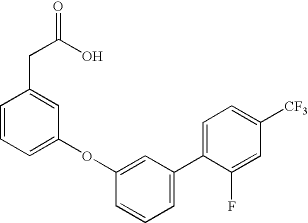 | 391.5 |
| P-0095 | 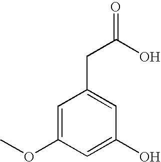 | 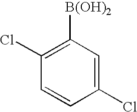 | 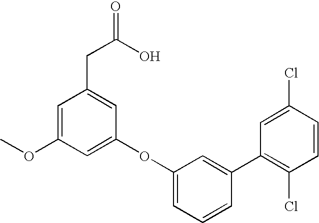 | 403.1 |
| P-0096 | 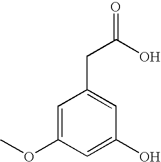 | 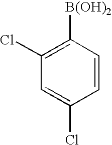 | 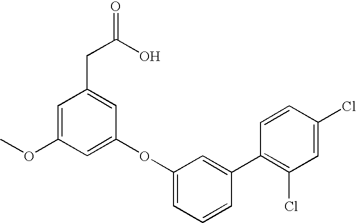 | 403.1 |
| P-0097 | 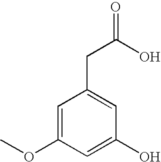 | 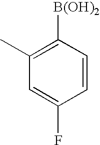 | 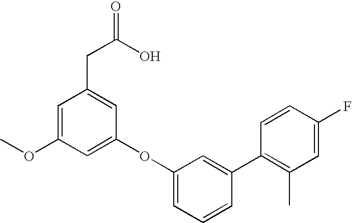 | 367.1 |
| P-0098 | 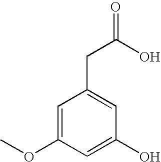 | 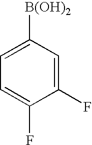 | 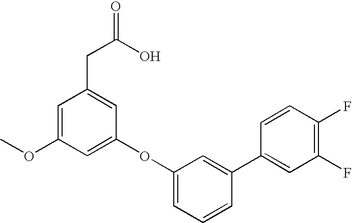 | 371.1 |
| P-0099 | 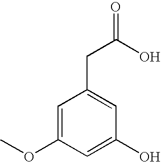 | 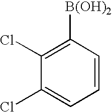 | 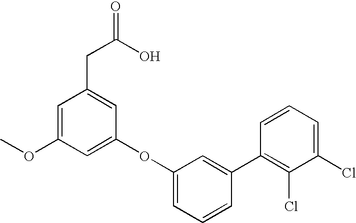 | 403.1 |
| P-0100 | 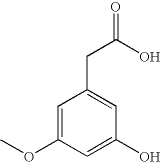 | 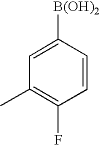 | 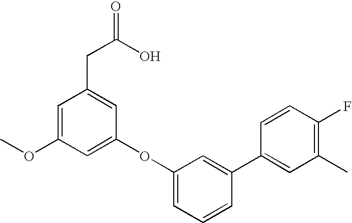 | 367.1 |
| P-0101 | 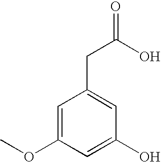 | 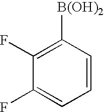 | 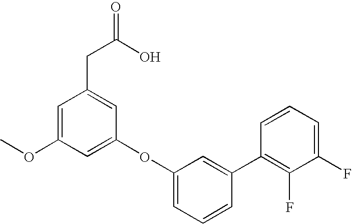 | 371.1 |
| P-0102 | 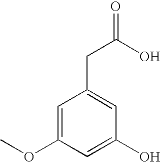 | 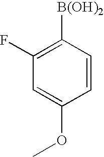 | 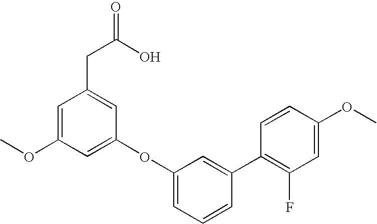 | 383.1 |
| P-0103 | 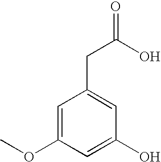 | 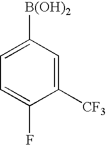 | 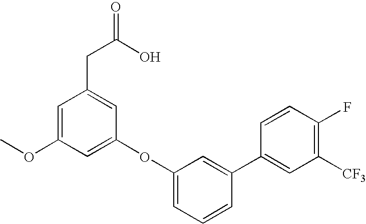 | 421.1 |
| P-0104 | 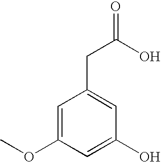 | 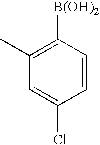 | 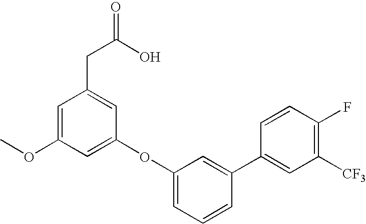 | 383.1 |
| P-0105 | 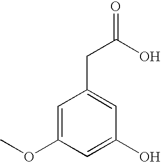 | 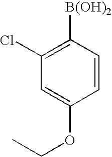 | 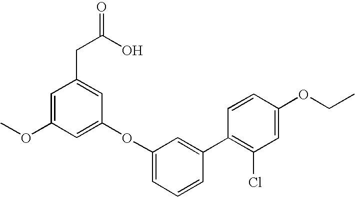 | 413.1 |
| P-0106 | 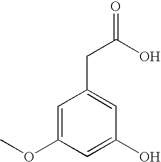 | 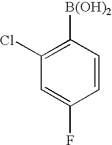 | 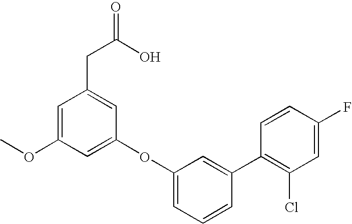 | 387.1 |
| P-0107 | 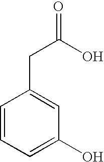 | 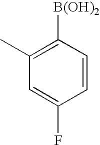 | 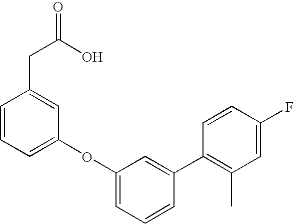 | 337.5 |
| P-0108 | 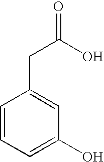 | 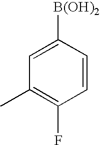 | 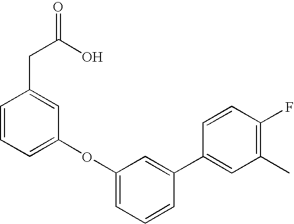 | 337.1 |
| P-0109 | 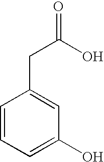 | 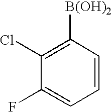 | 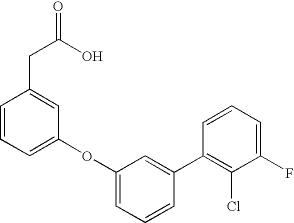 | 357.9 |
| P-0110 | 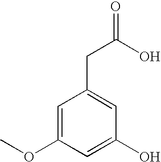 | 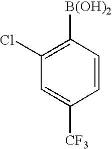 | 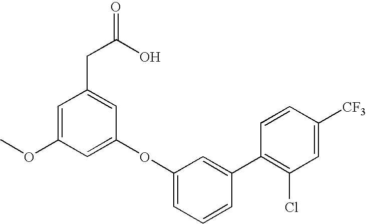 | 437.1 |
| P-0111 | 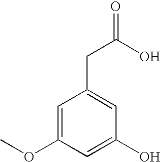 | 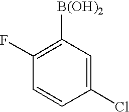 | 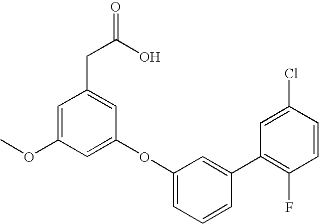 | 387.1 |
| P-0112 | 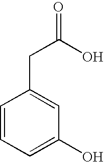 | 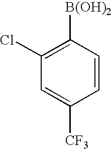 | 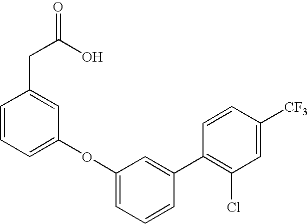 | 407.1 |
| P-0113 | 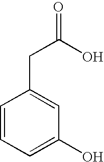 | 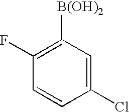 | 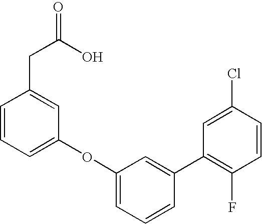 | 357.1 |
| P-0114 | 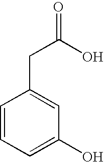 | 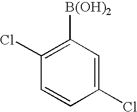 | 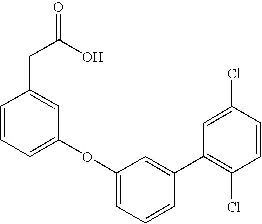 | 373.1 |
| P-0115 | 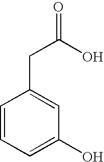 | 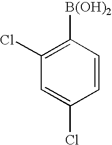 | 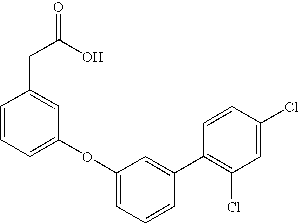 | 451.1(+DMSO) |
| P-0116 | 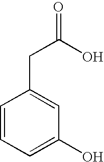 | 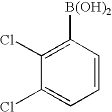 | 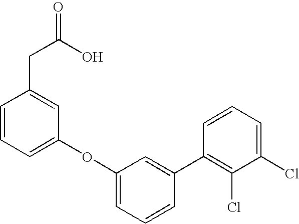 | 450.7(+DMSO) |
| P-0117 | 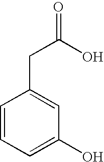 | 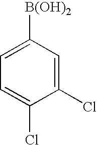 | 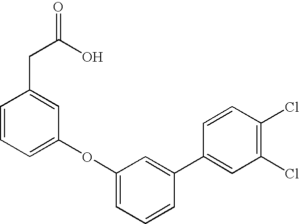 | 451.1(+DMSO) |
| P-0118 | 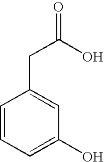 | 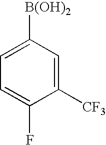 | 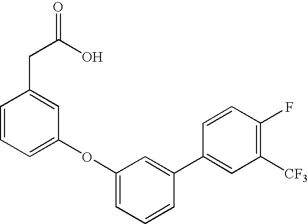 | 469.1(+DMSO) |
| P-0119 | 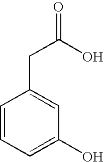 | 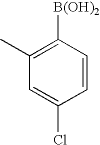 | 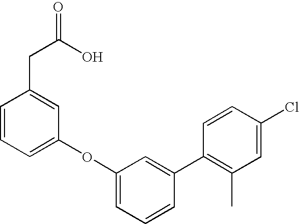 | 431.1(+DMSO) |
| P-0120 | 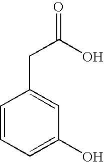 | 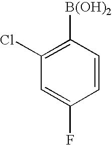 | 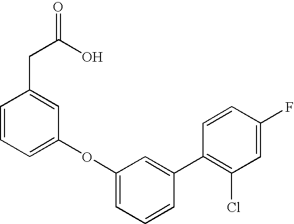 | 435.5(+DMSO) |
| P-0121 | 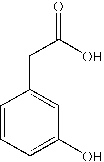 | 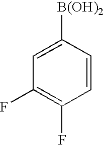 | 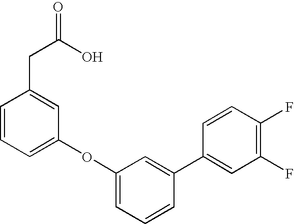 | 419.1(+DMSO) |
| P-0122 | 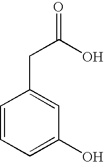 | 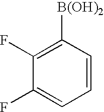 | 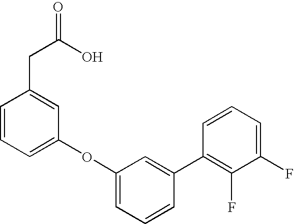 | 419.1(+DMSO) |
| P-0123 | 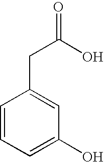 | 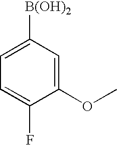 | 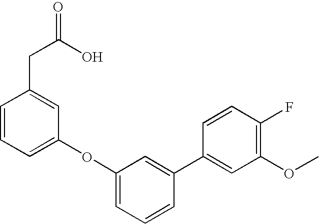 | 431.5(+DMSO) |
| P-0124 | 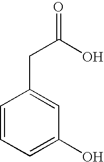 | 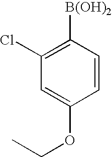 | 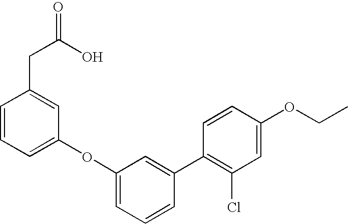 | 461.1(+DMSO) |
| P-0125 | 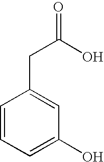 | 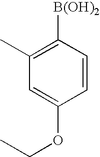 | 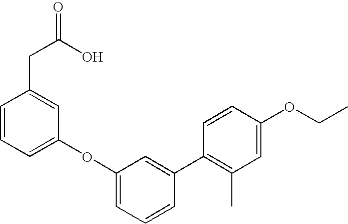 | 441.1(+DMSO) |

### Example 4

### Synthesis of [3-methyl-5-(4′-trifluoromethyl-biphenyl-3-sulfonyl)-phenyl]-acetic acid P-0089

[3-(4′-Chloro-biphenyl-3-yloxy)-5-methoxy-phenyl]-acetic acid P-0039 was synthesized in five steps from 3,5-dimethylphenol 9 as shown in Scheme 3.

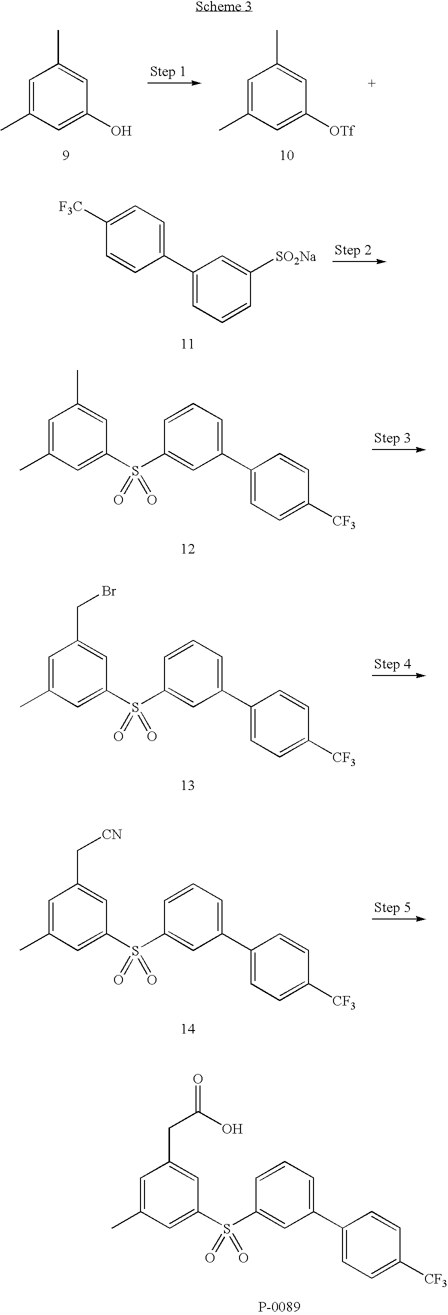

### Step 1—Preparation of trifluoro-methanesulfonic acid 3,5-dimethyl-phenyl ester (10)

Into a round bottom flask, 3,5-dimethylphenol (9, 1 equivalent) was dissolved in pyridine (80 equivalent). Trifluoroacetic anhydride (1.5 equivalent) was added dropwise. The reaction was allowed to stir at ambient conditions for 16 hours. The reaction was acidified with 2-3 mL of concentrated HCl and diluted with water, then the aqueous layer was extracted 3× with diethyl ether. The combined organic layers were washed 2× with 1M HCl, 2× with brine, and dried over sodium sulfate. Evaporation of solvent gave a yellow colored oily residue, which was used in the next step without further purification. ¹H NMR consistent with compound structure.

### Step 2—Preparation of 3-(3,5-dimethyl-benzenesulfonyl)-4′-trifluoromethyl-biphenyl (12)

Into a round bottom flask, trifluoro-methanesulfonic acid 3,5-dimethyl-phenyl ester (10, 0.30 g, 0.0012 mol), 4′-trifluoromethyl biphenyl-3-sulfinic acid sodium salt (11, 0.51 g, 0.0016 mol), Xanthphos (0.07 g, 0.0001 mol), cesium carbonate (0.54 g, 0.0016 mol), tris(dibenzylideneacetone)dipalladium (0) (0.1 g, 0.0001 mol), and 6 mL of toluene were combined and heated at 110° C. for 5 hours. The reaction was allowed to cool to room temperature and diluted with water. The reaction was extracted 4× with ethyl acetate. The combined organic layers were washed 2× with water, 1× with brine, and dried over sodium sulfate. Evaporation of solvent gave a yellow-orange oil. The oil was then purified via flash chromatography (20-30% ethyl acetate in hexane) and the solvent evaporated to yield the desired compound as a yellow oil. ¹H NMR consistent with compound structure.

### Step 3—Preparation of 3-(3-bromomethyl-5-methyl-benzenesulfonyl)-4′-trifluoromethyl-biphenyl (13)

Into a flask, 3-(3,5-dimethyl-benzenesulfonyl)-4′-trifluoromethyl-biphenyl (12, 100 mg, 1.1 mmol) was dissolved in 30 mL of carbon tetrachloride. N-bromosuccinimide (55 mg, 1.2 equivalent) and benzoyl peroxide (10 mg) were added, and the reaction was heated at 76° C. for 48 hours. The reaction was filtered to remove succinimide and the reaction was diluted with water and extracted 3× with dichloromethane. The combined organic layers were washed 2× with water, 1× with brine, and dried over sodium sulfate. Evaporation of solvent gave a solid, which was absorbed onto silica and purified via flash chromatography with a gradient of 20-30% ethyl acetate in hexanes over 16 minutes to yield the desired compound as an off-white solid. ¹H NMR consistent with compound structure.

### Step 4—Preparation of [3-methyl-5-(4′-trifluoromethyl-biphenyl-3-sulfonyl)-phenyl]-acetonitrile (14)

Into a flask, 3-(3-bromomethyl-5-methyl-benzenesulfonyl)-4′-trifluoromethyl-biphenyl (13, 30 mg, 0.064 mmol) was dissolved in 10 mL of ethanol. Sodium cyanide (5 mg) was added to the flask and the reaction was heated at 80° C. for 6 hours. The reaction was allowed to cool to room temperature, then was diluted with water and extracted 3× with dichloromethane. The combined organic layers were washed 2× with brine, and dried over sodium sulfate. Evaporation of solvent gave an oily residue. The oily residue was absorbed onto silica and purified via flash chromatography with a gradient of 15-25% ethyl acetate in hexanes to yield the desired compound as an oily residue. ¹H NMR consistent with compound structure.

### Step 5—Preparation of [3-methyl-5-(4′-trifluoromethyl-biphenyl-3-sulfonyl)-phenyl]-acetic acid (P-0089)

Into a flask, [3-methyl-5-(4′-trifluoromethyl-biphenyl-3-sulfonyl)-phenyl]-acetonitrile 14 was dissolved in ethanol (50 equivalent). 50% KOH in water (v/v, 5 mL) was added, and the flask was heated to 50° C. for 4 hours. After allowing the flask to cool to ambient conditions, the reaction was diluted with water and acidified with 10% HCl to pH=1-2. The aqueous layer was extracted 3× with ethyl acetate. The combined organic layers were washed 2× with water, 1× with brine, and dried over sodium sulfate. Evaporation under reduced pressure yielded an oily residue. The oily residue was purified via plate chromatography with 3% methanol in dichloromethane to yield the pure compound as an off-white solid. ¹H NMR consistent with compound structure. Calculated molecular weight=434.44, MS(ESI) [M−H⁺]⁻=433.03.

### Example 5

### Synthesis of [3-chloro-5-(4′-trifluoromethyl-biphenyl-3-sulfonyl)-phenyl]-acetic acid P-0090

[3-chloro-5-(4′-trifluoromethyl-biphenyl-3-sulfonyl)-phenyl]-acetic acid P-0090 was synthesized in six steps from (3-bromo-5-chloro-phenyl)-methanol 15 as shown in Scheme 4.

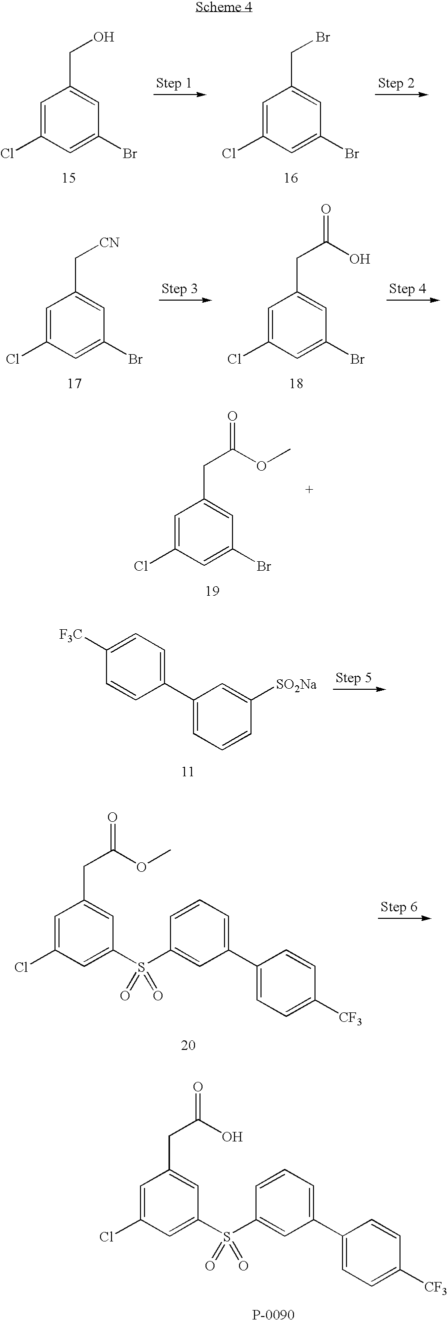

### Step 1—Preparation of 1-bromo-3-bromomethyl-5-chloro-benzene (16)

Into a flask, (3-bromo-5-chloro-phenyl)-methanol (15, 2400 mg, 0.011 mol) was dissolved in 200 mL of chloroform. Phosphorus tribromide in dichloromethane (1M, 16 mL) was added and the reaction mixture stirred overnight at ambient condition. The reaction mixture was diluted with water and extracted 3× with dichloromethane. The combined organic layers were washed 1× with water, 1× with brine, and dried over sodium sulfate. Evaporation of solvent gave the desired compound. ¹H NMR consistent with compound structure.

### Step 2—Preparation of (3-bromo-5-chloro-phenyl)-acetonitrile (17)

To a solution of 1-bromo-3-bromomethyl-5-chloro-benzene (16, 2.55 g, 0.00897 mol) in 50 mL of ethanol, sodium cyanide (570 mg, 0.012 mol) was added and the reaction mixture was refluxed overnight. The reaction mixture was concentrated, then diluted with water and the aqueous phase was extracted 2× with ether. The pooled organic phase was dried with sodium sulfate and concentrated in vacuo. The crude material was chromatographed on silica gel using 5% ethyl acetate in hexanes. ¹H NMR consistent with compound structure.

### Step 3—Preparation of (3-bromo-5-chloro-phenyl)-acetic acid (18)

To (3-bromo-5-chloro-phenyl)-acetonitrile (17, 700 mg, 0.003 mol), 700 mL of sulfuric acid, 700 mL of acetic acid, and 700 mL of water were added. The mixture was heated to reflux overnight. After the mixture cooled to ambient conditions, ethyl acetate and water were added. The phases were separated, and the organic phase was dried with sodium sulfate and the solvent evaporated. ¹H NMR consistent with compound structure. Calculated molecular weight=249.49, MS(ESI) [M−H⁺]⁻=248.9.

### Step 4—Preparation of (3-bromo-5-chloro-phenyl)-acetic acid methyl ester (19)

To a solution of (3-bromo-5-chloro-phenyl)-acetic acid (18, 0.753 g, 0.00302 mol) in 4 mL of methanol, 0.2 mL of sulfuric acid was added. The mixture was stirred overnight at room temperature, after which the mixture was concentrated in vacuo. Ethyl acetate and water were added and the layers were separated. The organic phase was washed twice with sat. NaHCO₃, then concentrated in vacuo. ¹H NMR consistent with compound structure.

### Step 5—Preparation of [3-chloro-5-(4′-trifluoromethyl-biphenyl-3-sulfonyl)-phenyl]-acetic acid (20)

(3-Bromo-5-Chloro-phenyl)-acetic acid methyl ester (19, 1 equivalent) and 4′-trifluoromethyl-biphenyl-3-sulfinic acid sodium salt (11, 1.2 equivalent) were put in a reaction vessel in toluene (60 equivalent). Tris(dibenzylideneacetone)dipalladium(0) (0.1 equivalent), cesium carbonate (1.5 equivalent), and Xanthphos (0.2 equivalent) were added to the vessel under an atmosphere of argon. The mixture was heated at 120° C. overnight. After cooling, the reaction mixture was diluted with water and extracted 2× with ethyl acetate. The combined organic layers were washed 1× with brine, dried over sodium sulfate and concentrated in vacuo. The crude material was absorbed onto silica and purified via flash chromatography with a gradient of 10-20% ethyl acetate in hexanes to give the desired compound. ¹H NMR consistent with compound structure.

### Step 6—Preparation of [3-chloro-5-(4′-trifluoromethyl-biphenyl-3-sulfonyl)-phenyl]-acetic acid (P-0090)

Into a flask, the [3-chloro-5-(4′-trifluoromethyl-biphenyl-3-sulfonyl)-phenyl]-acetic acid 20 was dissolved in tetrahydrofuran. 1M LiOH was added to the reaction to achieve a ratio of tetrahydrofuran:LiOH 4:1. The reaction was stirred overnight at ambient conditions. The reaction mixture was acidified using 1M HCl to pH=1-2. The reaction was diluted with water and extracted 2× with ethyl acetate. The combined organic layers were dried over sodium sulfate and evaporated under reduced pressure. The crude material was purified via flash chromatography with 1% methanol in dichloromethane to afford the desired compound. ¹H NMR consistent with compound structure.

### Example 6

### Synthesis of [3-(2′-fluoro-4′-trifluoromethyl-biphenyl-3-sulfonyl)-phenyl]-acetic acid P-0093

[3-(2′-Fluoro-4′-trifluoromethyl-biphenyl-3-sulfonyl)-phenyl]-acetic acid P-0093 was synthesized in six steps from 3-bromo-benzenesulfonyl chloride 21 as shown in Scheme 5.

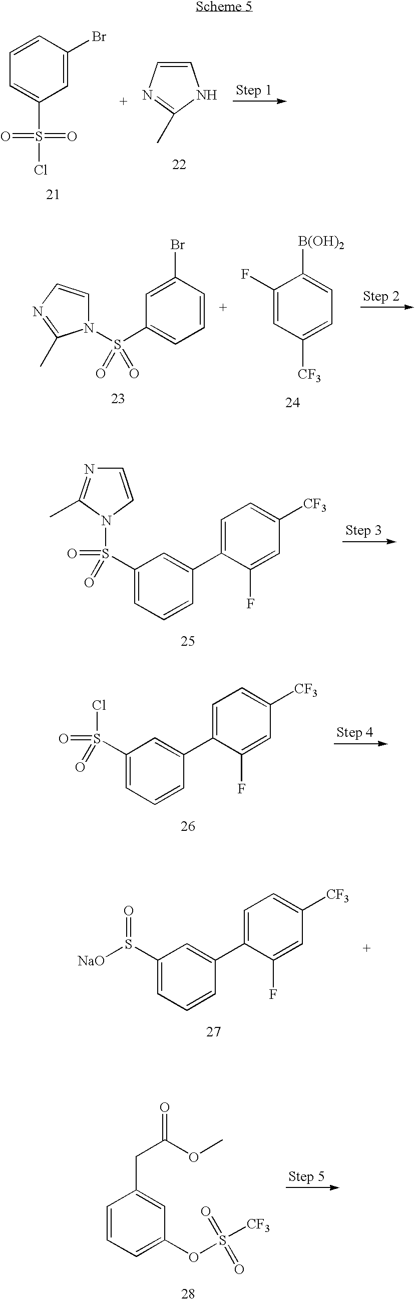

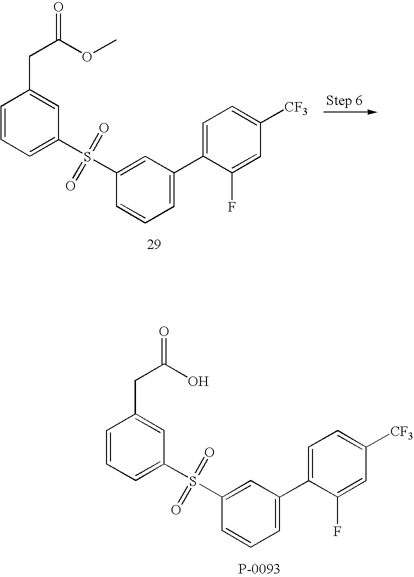

### Step 1—Preparation of 1-(3-bromo-benzenesulfonyl)-2-methyl-1H-imidazole (23)

Into a round bottom flask, 3-bromo-benzenesulfonyl chloride (21, 6 g, 0.02 mol), 2-methyl-1-imidazole (22, 2.1 g 0.026 mol), dichloromethane (80 mL, 1 mol), triethylamine (2 mL, 0.01 mol), and 4-dimethylaminopyridine (0.2 g, 0.002 mol) were combined and stirred under ambient conditions for 96 hours. The reaction was diluted with water, and the layers were separated. The aqueous layer was extracted 3× with dichloromethane, the combined organic layers were washed 2× with brine, dried over sodium sulfate, and evaporated under reduced pressure to afford a yellow oil. The oil was subjected to flash chromatography with 20% ethyl acetate (isocratic) in hexane to afford the desired compound 23 as a slightly yellow oil. ¹H NMR consistent with compound structure.

### Step 2—Preparation of 1-(2′-fluoro-4′-trifluoromethyl-biphenyl-3-sulfonyl)-2-methyl-1H-imidazole (25)

Into a round bottom flask, 1-(3-bromo-benzenesulfonyl)-2-methyl-1H-imidazole (23, 1.98 g, 0.00657 mol), 2-fluoro-4-trifluoromethyl phenyl boronic acid (24, 1.6 g, 0.0080 mol), tetrahydrofuran (81 mL, 1.0 mol), potassium carbonate in water (1 M, 30 mL), and tetrakis(triphenylphosphine)palladium(0) (0.5 g, 0.0004 mol) were combined and heated at 70° C. for 16 hours. The reaction was diluted with water and extracted 3× with ethyl acetate. The combined organic layers were washed 2× with brine, dried over sodium sulfate, and evaporated under reduced pressure to afford a yellow oil. The oil was absorbed onto silica and purified via flash chromatography with a gradient of 20-30% ethyl acetate in hexanes to afford the desired compound 25 as a lightly colored oil. ¹H NMR consistent with compound structure. MS(ESI) [M+H⁺]⁺=385.7.

### Step 3—Preparation of 2′-fluoro-4′-trifluoromethyl-biphenyl-3-sulfonyl chloride (26)

Into a round bottom flask, 1-(2′-fluoro-4′-trifluoromethyl-biphenyl-3-sulfonyl)-2-methyl-1H-imidazole (25, 0.5 g, 0.001 mol), methanol (5 mL, 0.1 mol), and potassium hydroxide in water (1 M, 5 mL) were combined and heated at 50° C. for 2 hours. The reaction was allowed to cool, and acidified with 10% HCl to pH=1-2, then frozen and freeze-dried to afford a white solid, which was dissolved with a solution of N,N-dimethylformamide (0.5 mL, 0.006 mol) in thionyl chloride (3 mL, 0.04 mol) (prepared separately at 0° C.) in a round bottom flask. The reaction was heated at 60° C. for 2.5 hours, then diluted with water and extracted 3× with ethyl acetate. The combined organic layers were washed 2× with water, then 2× with brine, and dried over sodium sulfate. Evaporation of solvent led to a dark oily residue. The oily residue was absorbed onto silica and purified via flash chromatography with 20-25% ethyl acetate in hexanes to yield compound 26 as an oily residue. ¹H NMR consistent with compound structure. MS(ESI) [M−H⁺]⁻319.1, which corresponds to sulfonic acid.

### Step 4—Preparation of sodium; 2′-fluoro-4′-trifluoromethyl-biphenyl-3-sulfinate (27)

Into a round bottom flask sodium sulfite (513 mg, 0.00407 mol) was dissolved in water (10 mL, 0.7 mol) at 90° C. 2′-Fluoro-4′-trifluoromethyl-biphenyl-3-sulfonyl chloride (26, 657 mg, 0.00194 mol) and sodium bicarbonate (360 mg, 0.0043 mol) were added simultaneously to the reaction. The reaction was heated at 90° C. for 3.5 hours, after which the reaction was cooled to room temperature and the solvent removed via freeze-drying to produce a white salt. Ethanol was added to the salt and the reaction was heated at 100° C. for 60 minutes, then subjected to hot filtration. The filtrate was evaporated under reduced pressure and the solid placed under a high vacuum to provide the desired compound 27.

### Step 5—Preparation of [3-(2′-fluoro-4′-trifluoromethyl-biphenyl-3-sulfonyl)-phenyl]-acetic acid methyl ester (29)

Into a round bottom flask, (3-trifluoromethanesulfonyloxy-phenyl)-acetic acid methyl ester (28, 0.200 g, 0.000671 mol, prepared as described in Example 2, Scheme 1, Step 2, replacing (3-methoxy-5-hydroxy-phenyl)-acetic acid methyl ester 2 with (3-hydroxy-phenyl)-acetic acid methyl ester), sodium; 2′-fluoro-4′-trifluoromethyl-biphenyl-3-sulfinate (27, 0.280 g, 0.000858 mol), xanthphos (0.08 g, 0.0001 mol), cesium carbonate (0.4 g, 0.001 mol), tris(dibenzylideneacetone)dipalladium(0) (0.07 g, 0.00007 mol), and toluene (6 mL, 0.06 mol) were combined and heated at 110° C. for 16 hours. The reaction was allowed to cool to room temperature, then diluted with water. The reaction was extracted 4× with ethyl acetate and the combined organic layers were washed with 2× water 2×, then 1× brine, and dried over sodium sulfate. Evaporation of solvent led to a yellow-orange oil. The oil was then purified via flash chromatography (20-30% ethyl acetate in hexane) to yield the desired compound 29 as a yellow oil. ¹H NMR consistent with compound structure.

### Step 6—Preparation of [3-(2′-fluoro-4′-trifluoromethyl-biphenyl-3-sulfonyl)-phenyl]-acetic acid (P-0093)

The [3-(2′-fluoro-4′-trifluoromethyl-biphenyl-3-sulfonyl)-phenyl]-acetic acid methyl ester 29 was dissolved in tetrahydrofuran (4 mL, 0.05 mol) and treated with potassium hydroxide in water (1 M, 2 mL) for 16 hours before workup. The reaction was acidified with 10% HCl to pH 1-2, and extracted 4× with ethyl acetate. The combined organic layers were washed 1× with brine and dried over sodium sulfate. Evaporation of solvent led to a yellow oil. The oil was then purified via flash chromatography at 9% methanol in dichloromethane to afford the desired compound P-0093 as a lightly yellow oil, which upon drying under high vacuum gave a white solid. ¹H NMR consistent with compound structure. MS(ESI) [M−H⁺]⁻=437.6.

[3-(2′-Chloro-4′-trifluoromethyl-biphenyl-3-sulfonyl)-5-methoxy-phenyl]-acetic acid P-0091, [3-(2′-Fluoro-4′-trifluoromethyl-biphenyl-3-sulfonyl)-5-methoxy-phenyl]-acetic acid P-0092, and [3-(2′-Chloro-4′-trifluoromethyl-biphenyl-3-sulfonyl)-phenyl]-acetic acid P-0094,

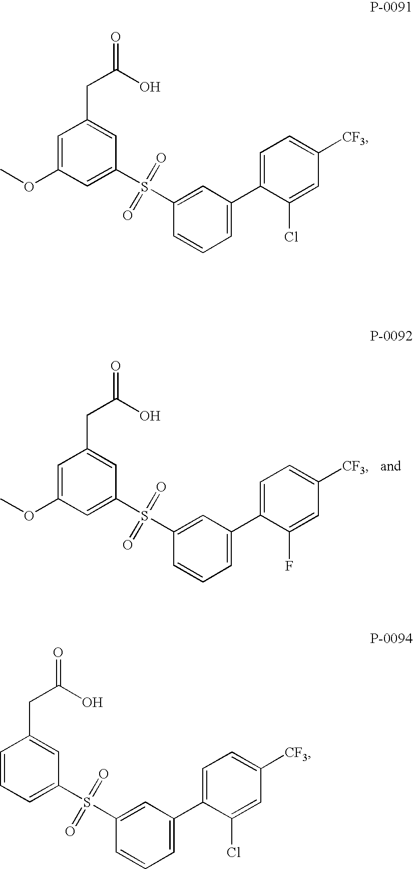

were prepared following the protocol of Scheme 5, replacing 2-fluoro-4-trifluoromethyl phenyl boronic acid 24 with 2-chloro-4-trifluoromethyl phenyl boronic acid in Step 2 and replacing (3-trifluoromethanesulfonyloxy-phenyl)-acetic acid methyl ester 28 with (3-methoxy-5-trifluoromethanesulfonyloxy-phenyl)-acetic acid methyl ester in Step 5 to provide P-0091 (MS(ESI) [M−H⁺]⁻=482.9); replacing (3-trifluoromethanesulfonyloxy-phenyl)-acetic acid methyl ester 28 with (3-methoxy-5-trifluoromethanesulfonyloxy-phenyl)-acetic acid methyl ester in Step 5 to provide P-0092 (MS(ESI) [M−H⁺]⁻=467.0); and replacing 2-fluoro-4-trifluoromethyl phenyl boronic acid 24 with 2-chloro-4-trifluoromethyl phenyl boronic acid in Step 2 to provide P-0094 (MS(ESI) [M−H⁺]⁻=453.5).

### Example 7

### PPAR Activity Assays

Assays for the activity of PPARα, PPARγ and PPARδ are known in the art, for example, biochemical and cell based assays as described in US Patent Application Publication number US 2007/0072904, the disclosure of which is hereby incorporated by reference in its entirety. Compounds having EC₅₀ of less than or equal to 1 μM in at least one of these assays, or a similar assay, for at least one of PPARα, PPARγ and PPARδ are shown in Table 3.

TABLE 3
Compounds of the invention having EC₅₀ of less than or equal to 1 μM in at least
one of PPARα PPARγ or PPARδ activity assays.

P-0003, P-0004, P-0016, P-0018, P-0021, P-0022, P-0023, P-0024, P-0025, P-0028, P-0030,
P-0031, P-0034, P-0036, P-0039, P-0042, P-0045, P-0046, P-0047, P-0048, P-0051, P-0058,
P-0063, P-0067, P-0068, P-0070, P-0071, P-0074, P-0076, P-0078, P-0079, P-0081, P-0082,
P-0083, P-0084, P-0085, P-0090, P-0091, P-0092, P-0093, P-0094, P-0095, P-0096, P-0097,
P-0098, P-0099, P-0100, P-0101, P-0102, P-0103, P-0104, P-0105, P-0106, P-0107, P-0110,
P-0111, P-0112, P-0115, P-0116, P-0117, P-0118, P-0119, P-0120, P-0121, P-0124, P-0125.

Additional examples of certain methods contemplated by the present invention may be found in the following applications: U.S. Prov. App. No. 60/715,214, filed Sep. 7, 2005, and U.S. Prov. App. No. 60/789,387, filed Apr. 5, 2006, and U.S. application Ser. No. 11/517,572, filed Sep. 6, 2006, all of which are incorporated herein by reference in their entireties including all specifications, figures, and tables, and for all purposes.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, and defined by the scope of the claims.

It will be readily apparent to one skilled in the art that varying substitutions and modifications may be made to the invention disclosed herein without departing from the scope and spirit of the invention. For example, variations can be made to provide additional compounds of Formula I and/or various methods of administration can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. Thus, for an embodiment of the invention using one of the terms, the invention also includes another embodiment wherein one of these terms is replaced with another of these terms. In each embodiment, the terms have their established meaning. Thus, for example, one embodiment may encompass a method “comprising” a series of steps, another embodiment would encompass a method “consisting essentially of” the same steps, and a third embodiment would encompass a method “consisting of” the same steps. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary where various numerical values are provided for embodiments, additional embodiments are described by taking any 2 different values as the endpoints of a range. Such ranges are also within the scope of the described invention.

Thus, additional embodiments are within the scope of the invention and within the following claims.

What is claimed is:

1. A compound having the chemical structure 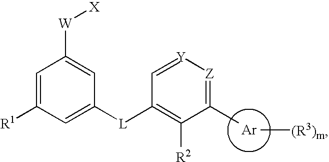
all salts, prodrugs, tautomers and isomers thereof,
wherein:
W is selected from the group consisting of a covalent bond, —NR⁴(CR⁵R⁶)₁₋₂—, —O—(CR⁵R⁶)₁₋₂—, —S—(CR⁵R⁶)₁₋₂—, —CHR⁶—, —(CR⁵R⁶)₂₋₃—, and —CR⁷═CR⁸—;
X is selected from the group consisting of —C(O)OR⁹, —C(O)NR¹⁰R¹¹, and a carboxylic acid isostere;
Y is CH or N;
Z is CH or N;
L is —NR⁴S(O)₂—, —S—, —S(O)—, —S(O)₂— or —O—;
Ar is aryl or heteroaryl;
R¹ is hydrogen, fluoro, chloro, methoxy, fluoro substituted methoxy, C₃₋₅ cycloalkyl, C₁₋₃ alkyl, or C₁₋₃ alkyl substituted with one or more fluoro, methoxy, or fluoro substituted methoxy;
R² is hydrogen, fluoro, chloro, C₁₋₃ alkyl or fluoro substituted C₁₋₃ alkyl;
R³ at each occurrence is independently selected from the group consisting of halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —NO₂, —CN, —OR¹², —NR¹²R¹³, —C(V)NR¹²R¹³, —C(V)R¹⁴, —S(O)₂NR¹²R¹³, —S(O)₂R¹⁴, —OC(V)R¹⁴, —C(V)OR¹², —C(NH)NR¹⁵R¹⁶, —NR¹²C(V)R¹⁴, —NR¹²S(O)₂R¹⁴, —NR¹²C(V)NR¹²R¹³, and —NR¹²S(O)₂NR¹²R¹³;
R⁴ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, provided, however, that any substitution on the alkyl carbon bound to the N of NR⁴ is fluoro, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁵ and R⁶ at each occurrence are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio;
R⁷ and R⁸ are independently hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio;
R⁹ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁹ is lower alkyl, any substitution on the alkyl carbon bound to the O of OR⁹ is fluoro;
R¹⁰ and R¹¹ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁰ and or R¹¹ is lower alkyl, any substitution on the alkyl carbon bound to the N of NR¹⁰R¹¹ is fluoro; or
R¹⁰ and R¹¹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R¹², R¹³, R¹⁵, and R¹⁶ at each occurrence are independently selected from the group consisting of hydrogen, optionally substituted lower alkyl, optionally substituted C₃₋₆ alkenyl, provided, however, that when R¹², R¹³, R¹⁵, or R¹⁶ is optionally substituted C₃₋₆ alkenyl, no alkene carbon thereof is bound to the O of any OR¹² or N of any NR¹², NR¹³, NR¹⁵ or NR¹⁶; optionally substituted C₃₋₆ alkynyl, provided, however, that when R¹², R¹³, R¹⁵, or R¹⁶ is optionally substituted C₃₋₆ alkynyl, no alkyne carbon thereof is the O of any OR¹² or N of any NR¹², NR¹³, NR¹⁵ or NR¹⁶; optionally substituted cycloalkyl, optionally substituted heterocycloalkyl, optionally substituted aryl, and optionally substituted heteroaryl, or
R¹⁵ and R¹⁶ combine with the nitrogen to which they are attached to form a 5-7 membered optionally substituted heterocycloalkyl or a 5 or 7 membered optionally substituted nitrogen containing heteroaryl;
R¹⁴ at each occurrence is independently selected from the group consisting of optionally substituted lower alkyl, optionally substituted C₃₋₆ alkenyl, provided, however, that when R¹⁴ is optionally substituted C₃₋₆ alkenyl, no alkene carbon thereof is bound to the S of any S(O)ₙR¹⁴ or the C of any C(Z)R¹⁴; optionally substituted C₃₋₆ alkynyl, provided, however, that when R¹⁴ is optionally substituted C₃₋₆ alkynyl, no alkyne carbon thereof is bound to the S of any S(O)ₙR¹⁴ or the C of any C(Z)R¹⁴; optionally substituted cycloalkyl, optionally substituted heterocycloalkyl, optionally substituted aryl, and optionally substituted heteroaryl;
V is O or S;
n is 0, 1 or 2; and
m is 0, 1, 2, 3, 4 or 5, provided, however, the compound is not 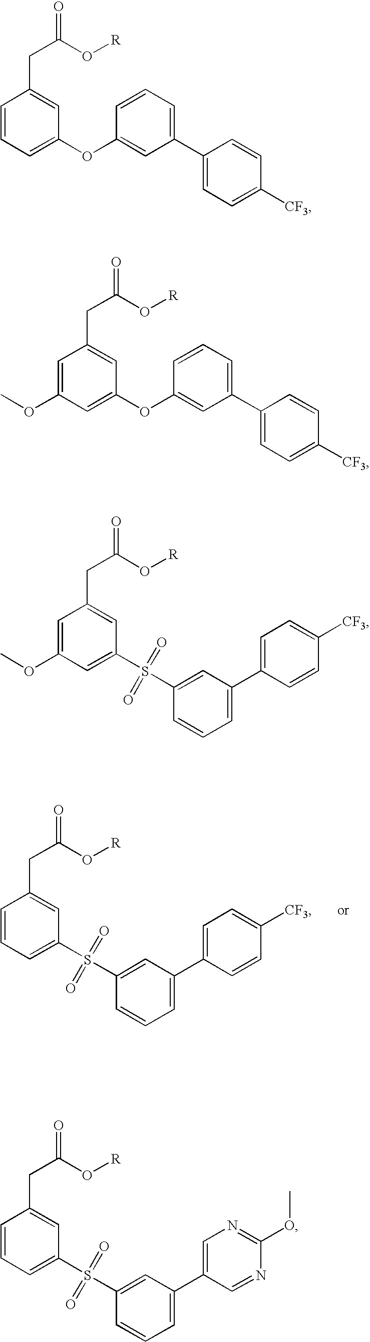
wherein R is H, methyl or ethyl.

2. The compound of claim 1, wherein W is —O—CR⁵R⁶—, —CHR⁶—, or —(CR⁵R⁶)₂—.

3. The compound of claim 2, wherein L is —O— or —S(O)₂—.

4. The compound of claim 3, wherein Ar is phenyl or monocyclic heteroaryl.

5. The compound of claim 4, wherein Ar is phenyl, pyridinyl, pyrimidinyl, pyrazolyl, imidazolyl, thiazolyl, isothiazolyl, oxazolyl, or isoxazolyl.

6. The compound of claim 5, wherein Ar is phenyl, pyridinyl, pyrimidinyl or pyrazolyl.

7. The compound of claim 1, wherein W is —CH₂—.

8. The compound of claim 7, having the structure of 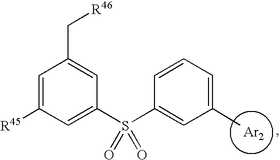
all salts, prodrugs, tautomers and isomers thereof,
wherein:
R⁴⁵ is hydrogen, chloro, methyl, or methoxy;
R⁴⁶ is selected from the group consisting of —C(O)OR⁴⁷, —C(O)NR⁴⁸R⁴⁹, and a carboxylic acid isostere;
R⁴⁷ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁴⁷ is lower alkyl, any substitution on the alkyl carbon bound to the O of OR⁴⁷ is fluoro;
R⁴⁸ and R⁴⁹ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁴⁸ and/or R⁴⁹ is lower alkyl, any substitution on the alkyl carbon bound to the N of NR⁴⁸R⁴⁹ is fluoro; or
R⁴⁸ and R⁴⁹ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
Ar₂ is selected from the group consisting of: 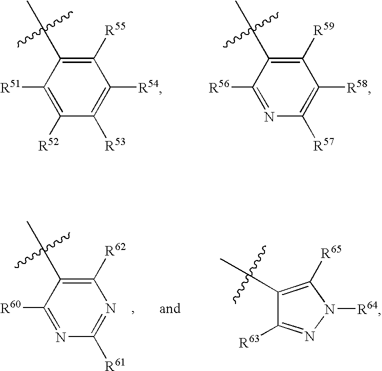
wherein 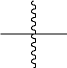
indicates the point of attachment of Ar₂ to the compound;
R⁵¹, R⁵², R⁵³, R⁵⁴, R⁵⁵, R⁵⁸ and R⁵⁹ are independently selected from the group consisting of hydrogen, fluoro, chloro, C₁₋₃ alkyl fluoro substituted C₁₋₃ alkyl, C₁₋₃ alkoxy, fluoro substituted C₁₋₃ alkoxy, and benzyloxy;
R⁵⁶, R⁵⁷, R⁶³ and R⁶⁵ are independently selected from the group consisting of hydrogen, fluoro, C₁₋₃ alkyl, fluoro substituted C₁₋₃ alkyl, C₁₋₃ alkoxy, fluoro substituted C₁₋₃ alkoxy, and benzyloxy;
R⁶⁰, R⁶¹ and R⁶² are independently selected from the group consisting of hydrogen, C₁₋₃ alkyl, fluoro substituted C₁₋₃ alkyl, C₁₋₃ alkoxy, fluoro substituted C₁₋₃ alkoxy, and benzyloxy; and
R⁶⁴ is lower alkyl or fluoro substituted lower alkyl.

9. The compound of claim 7, having the structure of 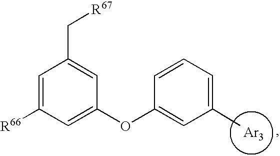
all salts, prodrugs, tautomers and isomers thereof,
wherein:
R⁶⁶ is hydrogen or methoxy;
R⁶⁷ is selected from the group consisting of —C(O)OR⁶⁸, —C(O)NR⁶⁹R⁷⁰, and a carboxylic acid isostere;
R⁶⁸ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁶⁸ is lower alkyl, any substitution on the alkyl carbon bound to the O of OR⁶⁸ is fluoro;
R⁶⁹ and R⁷⁰ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R⁶⁹ and/or R⁷⁰ is lower alkyl, any substitution on the alkyl carbon bound to the N of NR⁶⁹R⁷⁰ is fluoro; or
R⁶⁹ and R⁷⁰ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
Ar₃ is selected from the group consisting of: 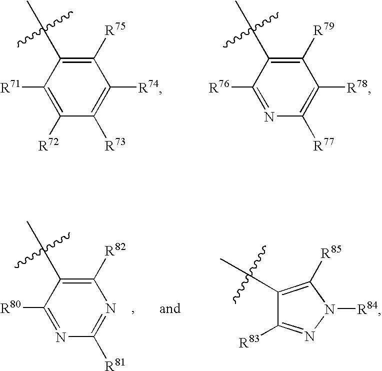
wherein 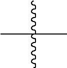
indicates the point of attachment of Ar₂ to the compound;
R⁷¹, R⁷², R⁷³, R⁷⁴, R⁷⁵, R⁷⁸ and R⁷⁹ are independently selected from the group consisting of hydrogen, fluoro, chloro, C₁₋₃ alkyl, fluoro substituted C₁₋₃ alkyl, C₁₋₃ alkoxy, fluoro substituted C₁₋₃ alkoxy, and benzyloxy;
R⁷⁶, R⁷⁷, R⁸³ and R⁸⁵ are independently selected from the group consisting of hydrogen, fluoro, C₁₋₃ alkyl, fluoro substituted C₁₋₃ alkyl, C₁₋₃ alkoxy, fluoro substituted C₁₋₃ alkoxy, and benzyloxy;
R⁸⁰, R⁸¹ and R⁸² are independently selected from the group consisting of hydrogen, C₁₋₃ alkyl, fluoro substituted C₁₋₃ alkyl, C₁₋₃ alkoxy, fluoro substituted C₁₋₃ alkoxy, and benzyloxy; and
R⁸⁴ is lower alkyl or fluoro substituted lower alkyl.

10. A composition comprising:
a pharmaceutically acceptable carrier; and
a compound according to claim 1.

11. A method for treating a subject suffering from or at risk of a disease or condition for which PPAR modulation provides a therapeutic benefit, comprising administering to said subject an effective amount of a compound according to claim 1.

12. A method for treating a subject suffering from or at risk of a disease or condition for which PPAR modulation provides a therapeutic benefit, comprising administering to said subject an effective amount of a composition according to claim 10.

13. The method according to claim 11, wherein said compound is approved for administration to a human.

14. The method according to claim 11, wherein said disease or condition is a PPAR-mediated disease or condition.

15. The method according to claim 11, wherein said disease or condition is selected from the group consisting of obesity, overweight condition, bulimia, anorexia nervosa, hyperlipidemia, dyslipidemia, hypoalphalipoproteinemia, hypentriglyceridemia, hypercholesterolemia, low HDL, Metabolic Syndrome, Type II diabetes mellitus, Type I diabetes, hyperinsulinemia, impaired glucose tolerance, insulin resistance, neuropathy, nephropathy, retinopathy, diabetic foot ulcer, bladder dysfunction, bowel dysfunction, diaphragmatic dysfunction, cataracts, hypertension, coronary heart disease, heart failure, congestive heart failure, atherosclerosis, arteriosclerosis, stroke, cerebrovascular disease, myocardial infarction, peripheral vascular disease, vitiligo, uveitis, optic neuritis, pemphigus foliaceus, pemphigoid, inclusion body myositis, polymyositis, dermatomyositis, scleroderma, Grave's disease, Hashimoto's disease, chronic graft versus host disease, ankylosing spondylitis, rheumatoid arthritis, inflammatory bowel disease, systemic lupus erythematosis, Sjogren's Syndrome, multiple sclerosis, asthma, chronic obstructive pulmonary disease, polycystic kidney disease, polycystic ovary syndrome, pancreatitis, nephritis, hepatitis, otitis, stomatitis, sinusitis, arteritis, temporal arteritis, giant cell arteritis, polymyalgia rheumatica, eczema, psoriasis, dermatitis, impaired wound healing, Alzheimer's disease, Parkinson's disease, amyotrophic lateral sclerosis, spinal cord injury, demyelinating disease, thrombosis, gastroesophageal reflux, appendicitis, diverticulitis, gastrointestinal ulcers, ileus, motility disorders, infarction of the large or small intestine, renal insufficiency, erectile dysfunction, urinary incontinence, neurogenic bladder, ophthalmic inflammation, conjunctivitis, keratoconjunctivitis, corneal inflammation, dry eye syndrome, macular degeneration, pathologic neovascularization, lyme disease, HCV infection, HIV infection, Helicobacter pylori infection, encephalitis, meningitis, neuropathic pain, inflammatory pain, chronic pain syndrome, fibromyalgia, infertility, breast cancer and thyroid cancer.

16. A kit comprising a compound according to claim 1.

17. A kit comprising a composition according to claim 10.

18. The kit according to claim 16, further comprising a written indication that said compound is approved for administering to a human.

19. The kit according to claim 16, wherein said compound is approved for treatment of a medical indication selected from the group consisting of obesity, overweight condition, bulimia, anorexia nervosa, hyperlipidemia, dyslipidemia, hypoalphalipoproteinemia, hypertriglyceridemia, hypercholesterolemia, low HDL, Metabolic Syndrome, Type II diabetes mellitus, Type I diabetes, hyperinsulinemia, impaired glucose tolerance, insulin resistance, neuropathy, nephropathy, retinopathy, diabetic foot ulcer, bladder dysfunction, bowel dysfunction, diaphragmatic dysfunction, cataracts, hypertension, coronary heart disease, heart failure, congestive heart failure, atherosclerosis, arteriosclerosis, stroke, cerebrovascular disease, myocardial infarction, peripheral vascular disease, vitiligo, uveitis, optic neuritis, pemphigus foliaceus, pemphigoid, inclusion body myositis, polymyositis, dermatomyositis, scleroderma, Grave's disease, Hashimoto's disease, chronic graft versus host disease, ankylosing spondylitis, rheumatoid arthritis, inflammatory bowel disease, systemic lupus erythematosis, Sjogren's Syndrome, multiple sclerosis, asthma, chronic obstructive pulmonary disease, polycystic kidney disease, polycystic ovary syndrome, pancreatitis, nephritis, hepatitis, otitis, stomatitis, sinusitis, arteritis, temporal arteritis, giant cell arteritis, polymyalgia rheumatica, eczema, psoriasis, dermatitis, impaired wound healing, Alzheimer's disease, Parkinson's disease, amyotrophic lateral sclerosis, spinal cord injury, demyelinating disease, thrombosis, gastroesophageal reflux, appendicitis, diverticulitis, gastrointestinal ulcers, ileus, motility disorders, infarction of the large or small intestine, renal insufficiency, erectile dysfunction, urinary incontinence, neurogenic bladder, ophthalmic inflammation, conjunctivitis, keratoconjunctivitis, corneal inflammation, dry eye syndrome, macular degeneration, pathologic neovascularization, lyme disease, HCV infection, HIV infection, Helicobacter pylori infection, encephalitis, meningitis, neuropathic pain, inflammatory pain, chronic pain syndrome, fibromyalgia, infertility, breast cancer and thyroid cancer.
